(19) United States
(12) Patent Application Publication
(10) Pub. No.: US 2011/0275683 A1
(43) Pub. Date: Nov. 10, 2011

(54) Selenophene and Selenazole Carboxylic Acid Derivatives

(75) Inventor: Regina Graul, Duxbury, MA (US)

(73) Assignee: Ironwood Pharmaceuticals, Inc, Cambridge, MA (US)

(21) Appl. No.: 12/995,967

(22) PCT Filed: Jun. 2, 2009
(86) PCT No.: PCT/US09/03336

§ 371 Date: Jul. 7, 2011

Related U.S. Application Data
(60) Provisional application No. 61/058,110, filed on Jun. 2, 2008.

(51) Int. Cl. A61K 31/407; A61P 25/00; A61P 25/18; A61P 25/24; C07D 517/04; A61P 25/28

(52) U.S. Cl. 514/382; 548/453; 548/252; 548/121; 514/412

(57) ABSTRACT

Derivatives of selenophene and selenazole heterocycles are disclosed. The compounds are useful as inhibitors of D-amino acid oxidase (DAO) and in the treatment of neurodegenerative and psychiatric diseases and disorders.

### PRIORITY CLAIM

This application claims priority to U.S. Application Ser. No. 61/058,110, filed Jun. 2, 2008. The entire contents of the aforementioned application is incorporated herein by reference.

### FIELD OF THE PRESENT DISCLOSURE

The present disclosure generally relates to bicyclic heterocycles, and more specifically to such compounds that are useful as inhibitors of D-amino acid oxidase (DAO) and in the treatment of neurodegenerative and psychiatric diseases and disorders.

### BACKGROUND

N-methyl-D-aspartate (NMDA)-glutamate receptors are expressed at excitatory synapses throughout the central nervous system (CNS). These receptors mediate a wide range of brain processes, including synaptic plasticity associated with certain forms of memory formation and learning. NMDA-glutamate receptors require binding of two agonists to affect neurotransmission. One of these agents is the excitatory amino acid L-glutamate, while the second agonist is thought to be D-serine. In animals D-serine is synthesized from L-serine by serine racemase and degraded to its corresponding keto acid by D-amino acid oxidase (DAO). Together, serine racemase and DAO are thought to play a crucial role in modulating NMDA receptor mediated neurotransmission by regulating CNS concentrations of D-serine. It is thought that inhibition of DAO will lead to increased D-serine levels and improved cognitive function. In addition to D-serine, DAO also degrades other amino acids and thus, DAO inhibitors may also modulate other DAO substrates providing therapeutic activity independent of NMDA receptor activation.

A limited set of compounds have been suggested as inhibitors of DAO, including certain heterocylic-2-carboxylic acids, might be useful for improving memory, learning and cognition in patients suffering from neurodegenerative disorders (U.S. Pub. Application No. 20030162825). Indomethacin has also been shown to be an inhibitor of DAO (Chen et al. 1994 Drug Metabol. Drug Interact. 11:153-60).

PCT patent application WO 03/039540 discloses pyrrole, indole, tetrahydrocyclopenta[c]quinoline and hexahydrocyclopenta[c]quinoline carboxylic acids derivatives which are said to be DAO inhibitors useful for improving learning and memory.

PCT patent application WO 07/39773 discloses thienopyrrole and furopyrrole carboxylic acids derivatives which are DAO inhibitors said to be useful for treating neurodegenerative and psychiatric diseases.

Accordingly, the present disclosure addresses the need in the art for additional inhibitors for DAO.

### SUMMARY OF THE PRESENT DISCLOSURE

Disclosed herein are a group of bicyclic selenium-containing heterocycles that have activity as DAO inhibitors and are useful in the treatment of neurodegenerative and psychiatric disorders and diseases.

In a first aspect, the present disclosure provides compounds of the formula:

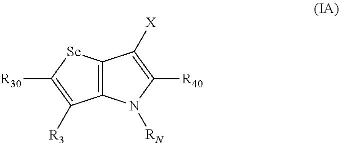

and pharmaceutically acceptable salts thereof, where
R_{N} is (i) hydrogen; (ii) C₁-C₆ alkylcarbonyl optionally substituted by one or two amino groups; (iii) —S(O)ₙR₆, —S(O)ₙNH₂, —S(O)ₙNH(R₆), or —S(O)ₙN(R₆)₂, where each R₆ is independently C₁-C₆ alkyl, C₂-C₆ alkenyl, or C₂-C₆ alkynyl, where each R₆ is optionally substituted with phenyl, and n is 1 or 2; (iv) C₁-C₆ alkyl optionally substituted with one or more groups which are independently halogen or hydroxy; (v) aryl(C₁-C₂)alkyl; or (vi) heteroaryl(C₁-C₂)alkyl, where the aryl and heteroaryl groups in (v) and (vi) are optionally substituted with one or more groups which are independently halogen, hydroxy, amino, nitro, C₁-C₆ alkylthio, hydroxy(C₁-C₆)alkyl, amino(C₁-C₆)alkyl, halo(C₁-C₆)alkyl, halo(C₁-C₆)alkoxy, or mono- or di(C₁-C₆)alkylamino;
R₄₀ is —COR or —R₅₀, where
   R is hydroxy, hydroxyamino, C₁-C₆ alkoxy, C₁-C₆ alkylcarbonyloxy, aryloxy, aryl(C₁-C₆)alkoxy, or —NR₁R₂, where R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or more groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, C₅-C₇ heterocycloalkyl, mono- or di(C₁-C₆)alkylamino, or carboxy; and
   R₅₀ is

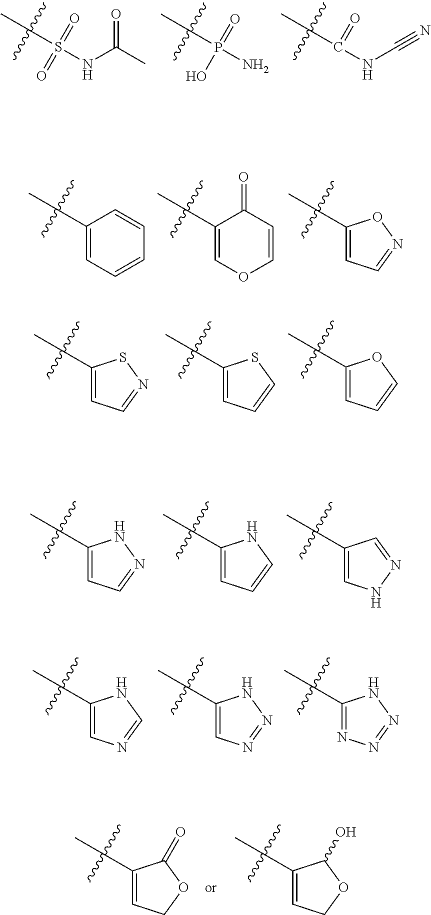

any of which may be optionally substituted with one or more groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy;
X is hydrogen, hydroxy, fluoro, chloro, bromo, cyano, nitro, C₁-C₆ alkyl, C₂-C₆ alkenyl, C₂-C₆ alkynyl, C₁-C₆ alkoxy, trifluoromethyl, or trifluoromethoxy;
R₃ is R₁₇ or R₁₈;
R₃₀ is R₁₈;
R₁₇ is —SR₅, —S(O)ₚR₅, —S(O)ₚNH₂, —S(O)ₚNHR₅, —S(O)ₚN(R₅)₂, COOH, C₁-C₁₀ alkyl, C₂-C₁₀ alkenyl, C₂-C₁₀ alkynyl, C₁-C₆ alkoxy, (C₁-C₆) alkylthio, halo(C₁-C₆)alkyl, halo(C₁-C₆)alkoxy, halo(C₁-C₆)alkylthio, or mono- or di(C₁-C₆)alkylamino, C₃-C₇ cycloalkyl, aryl, heteroaryl, aryl(C₁-C₆)alkyl, heteroaryl(C₁-C₆)alkyl, or C₅-C₇ heterocycloalkyl, where
   each cycloalkyl, heterocycloalkyl, aryl, and heteroaryl group is optionally substituted with one or more groups which are independently halogen, hydroxy, hydroxyamino, amino, nitro, cyano, C₁-C₆ alkyl, C₂-C₆ alkenyl, C₂-C₆ alkynyl, C₁-C₆ alkoxy, C₁-C₆ alkylthio, hydroxyalkyl, halo(C₁-C₃)alkyl, halo(C₁-C₃)alkoxy, halo(C₁-C₃)alkylthio, mono- or di(C₁-C₆)alkylamino, or amino(C₁-C₆)alkyl;
   R₅ is C₁-C₆ alkyl, C₂-C₆alkenyl, or C₂-C₆alkynyl, where R₅ is optionally substituted by phenyl, and
   p is 1 or 2; and
each R₁₈ is independently hydrogen, halogen, hydroxy, amino, hydroxyamino, nitro, cyano, —SR₅₁, —S(O)ₜR₅₁, —S(O)ₜNH₂, —S(O)ₜNHR₅₁, —S(O)ₜN(R₅₁)₂, —Si(R₅₁)₃, C₁-C₄ alkyl, C₂-C₄ alkenyl, C₂-C₄ alkynyl, C₁-C₄ alkoxy, (C₁-C₄)alkylthio, halo(C₁-C₄)alkyl, halo(C₁-C₄)alkoxy, halo(C₁-C₄)alkylthio, or mono- or di(C₁-C₄)alkylamino, where
   each R₅₁ is independently C₁-C₄ alkyl, C₂-C₄ alkenyl, or C₂-C₄alkynyl; and
   each t is independently 1 or 2; and
provided that the compound is not
(a) 4H-selenopheno[3,2-b]pyrrole-5-carboxylic acid;
(b) methyl 4H-selenopheno[3,2-b]pyrrole-5-carboxylate; and
(c) ethyl 4H-selenopheno[3,2-b]pyrrole-5-carboxylate.

In another embodiment of Formula IA, the present disclosure provides compounds of the formula:

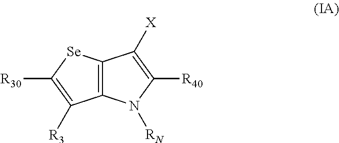

and pharmaceutically acceptable salts thereof, where
R_{N} is (i) hydrogen; (ii) C₁-C₆alkylcarbonyl optionally substituted by one or two amino groups; (iii) —S(O)ₙR₆, —S(O)ₙNH₂, —S(O)ₙNH(R₆), or —S(O)ₙN(R₆)₂, where each R₆ is independently C₁-C₆alkyl, C₂-C₆alkenyl, or C₂-C₆alkynyl, where each R₆ is optionally substituted with phenyl, and n is 1 or 2; (iv) C₁-C₆alkyl optionally substituted with one or more groups which are independently halogen or hydroxy; (v) aryl(C₁-C₂)alkyl; or (vi) heteroaryl(C₁-C₂)alkyl, where the aryl and heteroaryl groups in (v) and (vi) are optionally substituted with one or more groups which are independently halogen, hydroxy, amino, nitro, C₁-C₆alkylthio, hydroxy(C₁-C₆)alkyl, amino(C₁-C₆)alkyl, halo(C₁-C₆)alkyl, halo(C₁-C₆)alkoxy, or mono- or di(C₁-C₆)alkylamino;
R₄₀ is —COR or —R₅₀, where
   R is hydroxy, hydroxyamino, C₁-C₆alkoxy, C₁-C₆alkylcarbonyloxy, aryloxy, aryl(C₁-C₆)alkoxy, or —NR₁R₂, where R₁ and R₂ are independently (i) hydrogen, (ii) C₁-C₆alkyl, (iii) C₂-C₆alkenyl, (iv) C₂-C₆alkynyl, or (v) phenyl optionally substituted with one or more groups which are each independently halogen, hydroxy, amino, nitro, cyano, C₁-C₆alkyl, C₂-C₆alkenyl, C₂-C₆alkynyl, C₁-C₆alkoxy, C₁-C₆alkylthio, halo(C₁-C₆)alkyl, halo(C₁-C₆)alkoxy, halo(C₁-C₆) alkylthio, C₃-C₇cycloalkyl, C₅-C₇heterocycloalkyl, mono- or di(C₁-C₆)alkylamino, or carboxy; and
   R₅₀ is

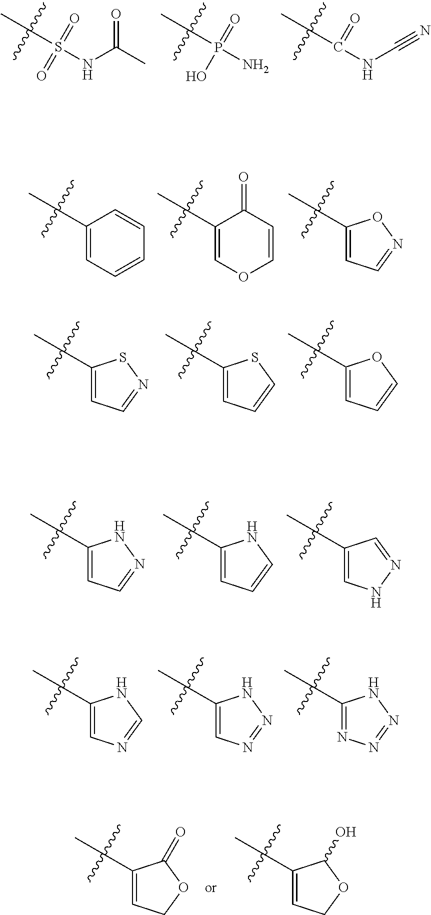

where each ring within R₅₀ is optionally substituted with one or more groups which are each independently halogen, hydroxy, nitro, C₁-C₆alkyl, C₂-C₆alkenyl, C₂-C₆alkynyl, halo(C₁-C₂)alkyl, or halo(C₁-C₂)alkoxy;
X is hydrogen, hydroxy, fluoro, chloro, cyano, nitro, C₁-C₆alkyl, C₂-C₆alkenyl, C₂-C₆alkynyl, C₁-C₆alkoxy, trifluoromethyl, or trifluoromethoxy;
R₃ is R₁₇ or R₁₈;
R₃₀ is R₁₈;
R₁₇ is —SR₅, —S(O)ₚR₅, —S(O)ₚNH₂, —S(O)ₚNHR₅, —S(O)ₚN(R₅)₂, C₁-C₁₀alkyl, C₂-C₁₀alkenyl, C₂-C₁₀alkynyl, C₁-C₆alkoxy, (C₁-C₆)alkylthio, halo(C₁-C₆)alkyl, halo(C₁-C₆)alkoxy, halo(C₁-C₆)alkylthio, or mono- or di(C₁-C₆)alkylamino, C₃-C₇cycloalkyl, aryl, heteroaryl, aryl(C₁-C₆)alkyl, heteroaryl(C₁-C₆)alkyl, or C₅-C₇heterocycloalkyl, where
   each cycloalkyl, heterocycloalkyl, aryl, and heteroaryl group is optionally substituted with one or more groups which are independently halogen, hydroxy, hydroxyamino, amino, nitro, cyano, C₁-C₆alkyl, C₂-C₆alkenyl, C₂-C₆alkynyl, C₁-C₆alkoxy, C₁-C₆alkylthio, hydroxyalkyl, halo(C₁-C₃)alkyl, halo(C₁-C₃)alkoxy, halo(C₁-C₃)alkylthio, mono- or di(C₁-C₆)alkylamino, or amino(C₁-C₆)alkyl;
   R₅ is C₁-C₆alkyl, C₂-C₆alkenyl, or C₂-C₆alkynyl, where R₅ is optionally substituted by phenyl, and
   p is 1 or 2; and
each R₁₈ is independently hydrogen, halogen, hydroxy, amino, hydroxyamino, nitro, cyano, —SR₅₁, —S(O)ₜR₅₁, —S(O)ₜNH₂, —S(O)ₜNHR₅₁, —S(O)ₜN(R₅₁)₂, C₁-C₄alkyl, C₂-C₄alkenyl, C₂-C₄alkynyl, C₁-C₄alkoxy, (C₁-C₄)alkylthio, halo(C₁-C₄)alkyl, halo(C₁-C₄)alkoxy, halo(C₁-C₄)alkylthio, or mono- or di(C₁-C₄)alkylamino, where
   each R₅₁ is independently C₁-C₄alkyl, C₂-C₄alkenyl, or C₂-C₄alkynyl; and
each t is independently 1 or 2; and
provided that the compound is not
(d) 4H-selenopheno[3,2-b]pyrrole-5-carboxylic acid;
(e) methyl 4H-selenopheno[3,2-b]pyrrole-5-carboxylate; and
(f) ethyl 4H-selenopheno[3,2-b]pyrrole-5-carboxylate.

In a second aspect, the present disclosure provides compounds of the formula:

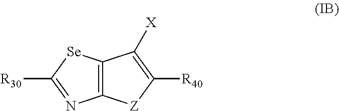

and pharmaceutically acceptable salts thereof, where
Z is —N(R_{N})— or —S—, where
   R_{N} is (i) hydrogen; (ii) C₁-C₆ alkylcarbonyl optionally substituted by one or two amino groups; (iii) —S(O)ₙR₆, —S(O)ₙNH₂, —S(O)ₙNH(R₆) or —S(O)ₙN(R₆)₂, where each R₆ is independently C₁-C₆ alkyl, C₂-C₆ alkenyl, or C₂-C₆ alkynyl, where each R₆ is optionally substituted with phenyl, and n is 1 or 2; (iv) C₁-C₆ alkyl optionally substituted with one or more groups which are independently halogen or hydroxy; (v) aryl(C₁-C₂)alkyl; or (vi) heteroaryl(C₁-C₂)alkyl, where the aryl and heteroaryl groups in (v) and (vi) are optionally substituted with one or more groups which are independently halogen, hydroxy, amino, nitro, C₁-C₆ alkylthio, hydroxy(C₁-C₆)alkyl, amino(C₁-C₆)alkyl, halo(C₁-C₆)alkyl, halo(C₁-C₆)alkoxy, or mono- or di(C₁-C₆)alkylamino;
R₄₀ is —COR or —R₅₀, where
   R is hydroxy, hydroxyamino, C₁-C₆ alkoxy, C₁-C₆ alkylcarbonyloxy, aryloxy, aryl(C₁-C₆)alkoxy, or —NR₁R₂, where R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or more groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, C₅-C₇ heterocycloalkyl, mono- or di(C₁-C₆)alkylamino, or carboxy; and
   R₅₀ is

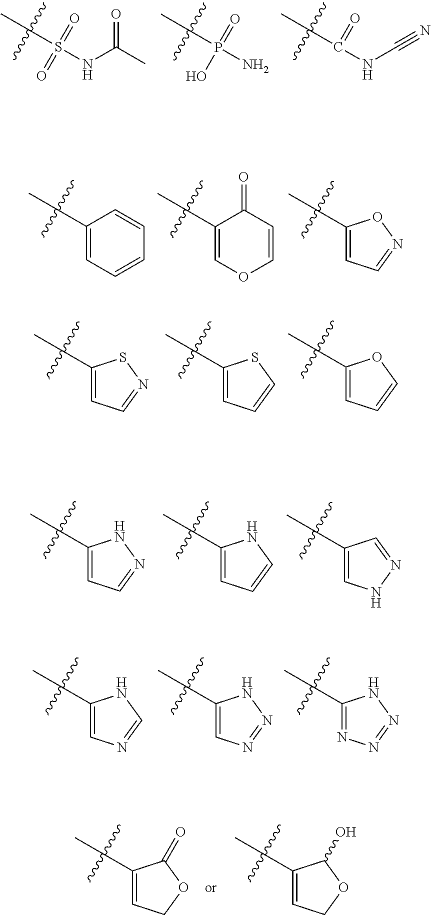

any of which may be optionally substituted with one or more groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy;
X is hydrogen, hydroxy, fluoro, chloro, bromo, cyano, nitro, C₁-C₆ alkyl, C₂-C₆ alkenyl, C₂-C₆ alkynyl, C₁-C₆ alkoxy, trifluoromethyl, or trifluoromethoxy;
and
R₃₀ is hydrogen, halogen, hydroxy, amino, hydroxyamino, nitro, cyano, —SR₅, —S(O)ₚR₅, —S(O)ₚNH₂, —S(O)ₚNHR₅, —S(O)ₚN(R₅)₂, —Si(R₅₁)₃, C₁-C₄ alkyl, C₂-C₄ alkenyl, C₂-C₄ alkynyl, C₁-C₄ alkoxy, (C₁-C₄)alkylthio, halo(C₁-C₄)alkyl, halo(C₁-C₄)alkoxy, halo(C₁-C₄)alkylthio, or mono- or di(C₁-C₄)alkylamino, where
   R₅ is C₁-C₄ alkyl, C₂-C₄alkenyl, or C₂-C₄alkynyl; and
   p is 1 or 2.

In another embodiment of Formula IB, the present disclosure provides compounds of the formula:

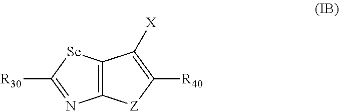

and pharmaceutically acceptable salts thereof, where
Z is —N(R_{N})— or —S—, where
   R_{N} is (i) hydrogen; (ii) C₁-C₆alkylcarbonyl optionally substituted by one or two amino groups; (iii) —S(O)ₙR₆, —S(O)ₙNH₂, —S(O)ₙNH(R₆), or —S(O)ₙN(R₆)₂, where each R₆ is independently C₁-C₉alkyl, C₂-C₆alkenyl, or C₂-C₆alkynyl, where each R₆ is optionally substituted with phenyl, and n is 1 or 2; (iv) C₁-C₆alkyl optionally substituted with one or more groups which are independently halogen or hydroxy; (v) aryl(C₁-C₂)alkyl; or (vi) heteroaryl(C₁-C₂)alkyl, where the aryl and heteroaryl groups in (v) and (vi) are optionally substituted with one or more groups which are independently halogen, hydroxy, amino, nitro, C₁-C₆alkylthio, hydroxy(C₁-C₆)alkyl, amino(C₁-C₆)alkyl, halo(C₁-C₆)alkyl, halo(C₁-C₆)alkoxy, or mono- or di (C₁-C₆)alkylamino;
R₄₀ is —COR or —R₅₀, where
   R is hydroxy, hydroxyamino, C₁-C₆alkoxy, C₁-C₆alkylcarbonyloxy, aryloxy, aryl(C₁-C₆)alkoxy, or —NR₁R₂, where R₁ and R₂ are independently (i) hydrogen, (ii) C₁-C₆alkyl, (iii) C₂-C₆alkenyl, (iv) C₂-C₆alkynyl, or (v) phenyl optionally substituted with one or more groups which are each independently halogen, hydroxy, amino, nitro, cyano, C₁-C₆alkyl, C₂-C₆alkenyl, C₂-C₆alkynyl, C₁-C₆alkoxy, C₁-C₆alkylthio, halo(C₁-C₆)alkyl, halo(C₁-C₆)alkoxy, halo(C₁-C₆) alkylthio, C₃-C₇cycloalkyl, C₅-C₇heterocycloalkyl, mono- or di(C₁-C₆)alkylamino, or carboxy; and
   R₅₀ is

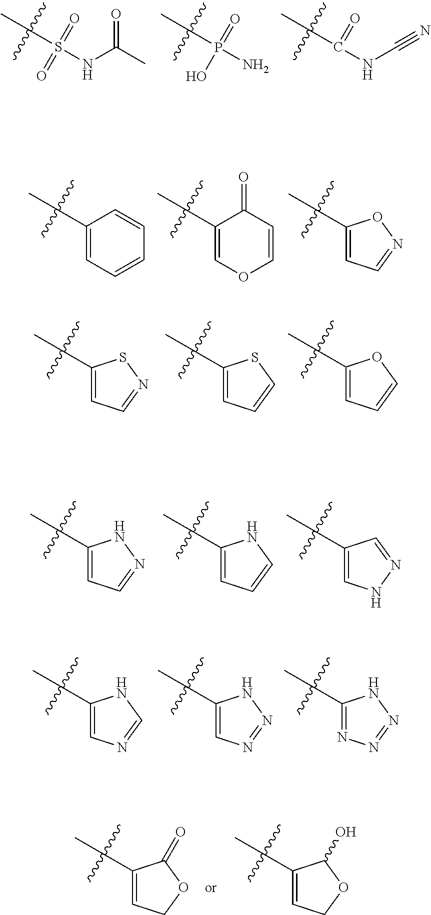

where each ring within R₅₀ is optionally substituted with one or more groups which are each independently halogen, hydroxy, nitro, C₁-C₆alkyl, C₂-C₆alkenyl, C₂-C₆alkynyl, halo(C₁-C₂)alkyl, or halo(C₁-C₂)alkoxy;
X is hydrogen, hydroxy, fluoro, chloro, cyano, nitro, C₁-C₆alkyl, C₂-C₆alkenyl, C₂-C₆alkynyl, C₁-C₆alkoxy, trifluoromethyl, or trifluoromethoxy;
and
R₃₀ is hydrogen, halogen, hydroxy, amino, hydroxyamino, nitro, cyano, —SR₅, —S(O)ₚR₅, —S(O)ₚNH₂, —S(O)ₚNHR₅, —S(O)ₚN(R₅)₂, C₁-C₄alkyl, C₂-C₄alkenyl, C₂-C₄alkynyl, C₁-C₄alkoxy, (C₁-C₄)alkylthio, halo(C₁-C₄)alkyl, halo(C₁-C₄)alkoxy, halo(C₁-C₄)alkylthio, or mono- or di (C₁-C₄)alkylamino, where
   R₅ is C₁-C₄alkyl, C₂-C₄alkenyl, or C₂-C₄alkynyl; and
   p is 1 or 2.

In a third aspect, the present disclosure provides compounds of the formula:

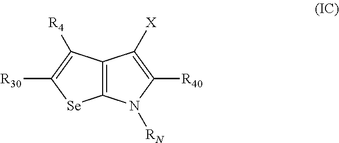

and pharmaceutically acceptable salts thereof, where
R_{N} is (i) hydrogen; (ii) C₁-C₆ alkylcarbonyl optionally substituted by one or two amino groups; (iii) —S(O)ₙR₆, —S(O)ₙNH₂, —S(O)ₙNH(R₆), or —S(O)ₙN(R₆)₂, where each R₆ is independently C₁-C₆ alkyl, C₂-C₆ alkenyl, or C₂-C₆ alkynyl, where each R₆ is optionally substituted with phenyl, and n is 1 or 2; (iv) C₁-C₆ alkyl optionally substituted with one or more groups which are independently halogen or hydroxy; (v) aryl(C₁-C₂)alkyl; or (vi) heteroaryl(C₁-C₂)alkyl, where the aryl and heteroaryl groups in (v) and (vi) are optionally substituted with one or more groups which are independently halogen, hydroxy, amino, nitro, C₁-C₆ alkylthio, hydroxy(C₁-C₆)alkyl, amino(C₁-C₆)alkyl, halo(C₁-C₆)alkyl, halo(C₁-C₆)alkoxy, or mono- or di(C₁-C₆)alkylamino;
R₄₀ is —COR or —R₅₀, where
   R is hydroxy, hydroxyamino, C₁-C₆ alkoxy, C₁-C₆ alkylcarbonyloxy, aryloxy, aryl(C₁-C₆)alkoxy, or —NR₁R₂, where R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or more groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, C₅-C₇ heterocycloalkyl, mono- or di(C₁-C₆)alkylamino, or carboxy; and
   R₅₀ is

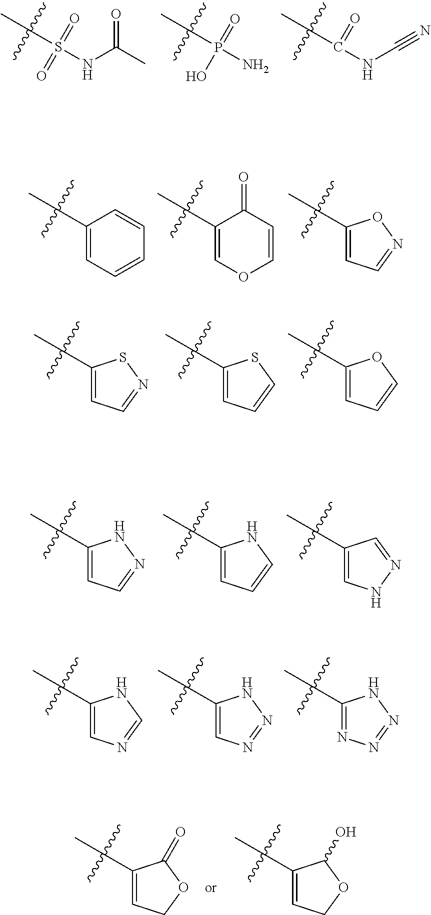

any of which may be optionally substituted with one or more groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy;
X is hydrogen, hydroxy, fluoro, chloro, bromo, cyano, nitro, C₁-C₆ alkyl, C₂-C₆ alkenyl, C₂-C₆ alkynyl, C₁-C₆ alkoxy, trifluoromethyl, or trifluoromethoxy;
R₄ is R₁₈ or R₁₇;
R₃₀ is R₁₈;
R₁₇ is —SR₅, —S(O)ₚR₅, —S(O)ₚNH₂, —S(O)ₚNHR₅, —S(O)ₚN(R₅)₂, COOH, C₁-C₁₀ alkyl, C₂-C₁₀ alkenyl, C₂-C₁₀ alkynyl, C₁-C₆ alkoxy, (C₁-C₆)alkylthio, halo(C₁-C₆)alkyl, halo(C₁-C₆)alkoxy, halo(C₁-C₆)alkylthio, or mono- or di(C₁-C₆)alkylamino, C₃-C₇ cycloalkyl, aryl, heteroaryl, aryl(C₁-C₆)alkyl, heteroaryl(C₁-C₆)alkyl, or C₅-C₇ heterocycloalkyl, where
   each cycloalkyl, heterocycloalkyl, aryl, and heteroaryl group is optionally substituted with one or more groups which are independently halogen, hydroxy, hydroxyamino, amino, nitro, cyano, C₁-C₆ alkyl, C₂-C₆ alkenyl, C₂-C₆ alkynyl, C₁-C₆ alkoxy, C₁-C₆ alkylthio, hydroxyalkyl, halo(C₁-C₃)alkyl, halo(C₁-C₃)alkoxy, halo(C₁-C₃)alkylthio, mono- or di(C₁-C₆)alkylamino, or amino(C₁-C₆)alkyl;
   R₅ is C₁-C₆ alkyl, C₂-C₆ alkenyl, or C₂-C₆ alkynyl, where each R₅ is optionally substituted by phenyl, and
   p is 1 or 2; and
each R₁₈ is independently hydrogen, halogen, hydroxy, amino, hydroxyamino, nitro, cyano, —SR₅₁, —S(O)ₜR₅₁, —S(O)ₜNH₂, —S(O)ₜNHR₅₁, —S(O)ₜN(R₅₁)₂, —Si (R₅₁)₃, C₁-C₄ alkyl, C₂-C₄ alkenyl, C₂-C₄ alkynyl, C₁-C₄ alkoxy, (C₁-C₄)alkylthio, halo(C₁-C₄)alkyl, halo(C₁-C₄)alkoxy, halo(C₁-C₄)alkylthio, or mono- or di(C₁-C₄)alkylamino, where
   each R₅₁ is independently C₁-C₄ alkyl, C₂-C₄ alkenyl, or C₂-C₄alkynyl; and
   each t is independently 1 or 2;
provided that
   (a) the compound is not
      (i) 6H-selenopheno[2,3-b]pyrrole-5-carboxylic acid; and
      (ii) ethyl
      6H-selenopheno[2,3-b]pyrrole-5-carboxylate;
   and
   (b) R⁴⁰ is not unsubstituted phenyl when R^{N} is methyl.

In another embodiment of Formula IC, the present disclosure provides compounds of the formula:

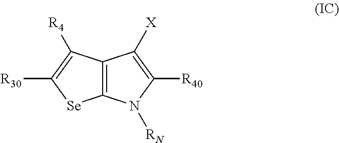

and pharmaceutically acceptable salts thereof, where
R_{N} is (i) hydrogen; (ii) C₁-C₆alkylcarbonyl optionally substituted by one or two amino groups; (iii) —S(O)ₙR₆, —S(O)ₙNH₂, —S(O)ₙNH(R₆), or —S(O)ₙN(R₆)₂, where each R₆ is independently C₁-C₆alkyl, C₂-C₆alkenyl, or C₂-C₆alkynyl, where each R₆ is optionally substituted with phenyl, and n is 1 or 2; (iv) C₁-C₆alkyl optionally substituted with one or more groups which are independently halogen or hydroxy; (v) aryl(C₁-C₂)alkyl; or (vi) heteroaryl(C₁-C₂)alkyl, where the aryl and heteroaryl groups in (v) and (vi) are optionally substituted with one or more groups which are independently halogen, hydroxy, amino, nitro, C₁-C₆alkylthio, hydroxy(C₁-C₆)alkyl, amino(C₁-C₆)alkyl, halo(C₁-C₆)alkyl, halo(C₁-C₆)alkoxy, or mono- or di(C₁-C₆)alkylamino;
R₄₀ is —COR or —R₅₀, where
   R is hydroxy, hydroxyamino, C₁-C₆alkoxy, C₁-C₆alkylcarbonyloxy, aryloxy, aryl(C₁-C₆)alkoxy, or —NR₁R₂, where R₁ and R₂ are independently (i) hydrogen, (ii) C₁-C₆alkyl, (iii) C₂-C₆alkenyl, (iv) C₂-C₆alkynyl, or (v) phenyl optionally substituted with one or more groups which are each independently halogen, hydroxy, amino, nitro, cyano, C₁-C₆alkyl, C₂-C₆alkenyl, C₂-C₆alkynyl, C₁-C₆alkoxy, C₁-C₆alkylthio, halo(C₁-C₆)alkyl, halo(C₁-C₆)alkoxy, halo(C₁-C₆)alkylthio, C₃-C₇cycloalkyl, C₅-C₇heterocycloalkyl, mono- or di(C₁-C₆)alkylamino, or carboxy; and
   R₅₀ is

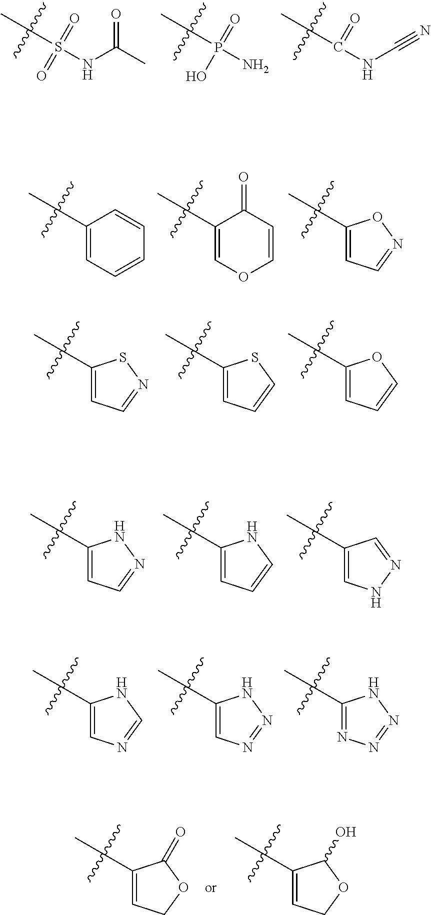

where each ring within R₅₀ is optionally substituted with one or more groups which are each independently halogen, hydroxy, nitro, C₁-C₆alkyl, C₂-C₆alkenyl, C₂-C₆alkynyl, halo(C₁-C₂)alkyl, or halo(C₁-C₂)alkoxy;
X is hydrogen, hydroxy, fluoro, chloro, cyano, nitro, C₁-C₆alkyl, C₂-C₆alkenyl, C₂-C₆alkynyl, C₁-C₆alkoxy, trifluoromethyl, or trifluoromethoxy;
R₄ is R₁₈ or R₁₇;
R₃₀ is R₁₈;
R₁₇ is —SR₅, —S(O)ₚR₅, —S(O)ₚNH₂, —S(O)ₚNHR₅, —S(O)ₚN(R₅)₂, C₁-C₁₀alkyl, C₂-C₁₀alkenyl, C₂-C₁₀alkynyl, C₁-C₆alkoxy, (C₁-C₆)alkylthio, halo(C₁-C₆)alkyl, halo(C₁-C₆)alkoxy, halo(C₁-C₆)alkylthio, or mono- or di (C₁-C₆)alkylamino, C₃-C₇cycloalkyl, aryl, heteroaryl, aryl(C₁-C₆)alkyl, heteroaryl (C₁-C₆)alkyl, or C₅-C₇heterocycloalkyl, where
   each cycloalkyl, heterocycloalkyl, aryl, and heteroaryl group is optionally substituted with one or more groups which are independently halogen, hydroxy, hydroxyamino, amino, nitro, cyano, C₁-C₆alkyl, C₂-C₆alkenyl, C₂-C₆alkynyl, C₁-C₆alkoxy, C₁-C₆alkylthio, hydroxyalkyl, halo(C₁-C₃)alkyl, halo(C₁-C₃)alkoxy, halo(C₁-C₃)alkylthio, mono- or di (C₁-C₆)alkylamino, or amino(C₁-C₆)alkyl;
   R₅ is C₁-C₆alkyl, C₂-C₆alkenyl, or C₂-C₆alkynyl, where each R₅ is optionally substituted by phenyl, and
   p is 1 or 2; and
each R₁₈ is independently hydrogen, halogen, hydroxy, amino, hydroxyamino, nitro, cyano, —SR₅₁, —S(O)ₜR₅₁, —S(O)ₜNH₂, —S(O)ₜNHR₅₁, —S(O)ₜN(R₅₁)₂, C₁-C₄alkyl, C₂-C₄alkenyl, C₂-C₄alkynyl, C₁-C₄alkoxy, (C₁-C₄)alkylthio, halo(C₁-C₄)alkyl, halo(C₁-C₄)alkoxy, halo(C₁-C₄)alkylthio, or mono- or di(C₁-C₄)alkylamino, where
   each R₅₁ is independently C₁-C₄alkyl, C₂-C₄alkenyl, or C₂-C₄alkynyl; and
   each t is independently 1 or 2;
provided that
   (a) the compound is not
      (i) 6H-selenopheno[2,3-b]pyrrole-5-carboxylic acid; and
      (ii) ethyl
      6H-selenopheno[2,3-b]pyrrole-5-carboxylate;
   and
   (b) R⁴⁰ is not unsubstituted phenyl when R^{N} is methyl.

In a fourth aspect, the present disclosure provides compounds of the formula:

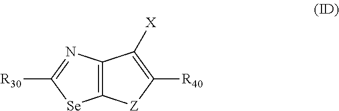

and pharmaceutically acceptable salts thereof, where
Z is —N(R_{N})— or —S—, where
   R_{N} is (i) hydrogen; (ii) C₁-C₆ alkylcarbonyl optionally substituted by one or two amino groups; (iii) —S(O)ₙR₆, —S(O)ₙNH₂, —S(O)ₙNH(R₆) or —S(O)ₙN(R₆)₂, where each R₆ is independently C₁-C₆ alkyl, C₂-C₆ alkenyl, or C₂-C₆ alkynyl, where each R₆ is optionally substituted with phenyl, and n is 1 or 2; (iv) C₁-C₆ alkyl optionally substituted with one or more groups which are independently halogen or hydroxy; (v) aryl(C₁-C₂)alkyl; or (vi) heteroaryl(C₁-C₂)alkyl, where the aryl and heteroaryl groups in (v) and (vi) are optionally substituted with one or more groups which are independently halogen, hydroxy, amino, nitro, C₁-C₆ alkylthio, hydroxy(C₁-C₆)alkyl, amino(C₁-C₆)alkyl, halo(C₁-C₆)alkyl, halo(C₁-C₆)alkoxy, or mono- or di(C₁-C₆)alkylamino;
R₄₀ is —COR or —R₅₀, where
   R is hydroxy, hydroxyamino, C₁-C₆ alkoxy, C₁-C₆ alkylcarbonyloxy, aryloxy, aryl(C₁-C₆)alkoxy, or —NR₁R₂ where R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or more groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, C₅-C₇ heterocycloalkyl, mono- or di(C₁-C₆)alkylamino, or carboxy; and
   R₅₀ is

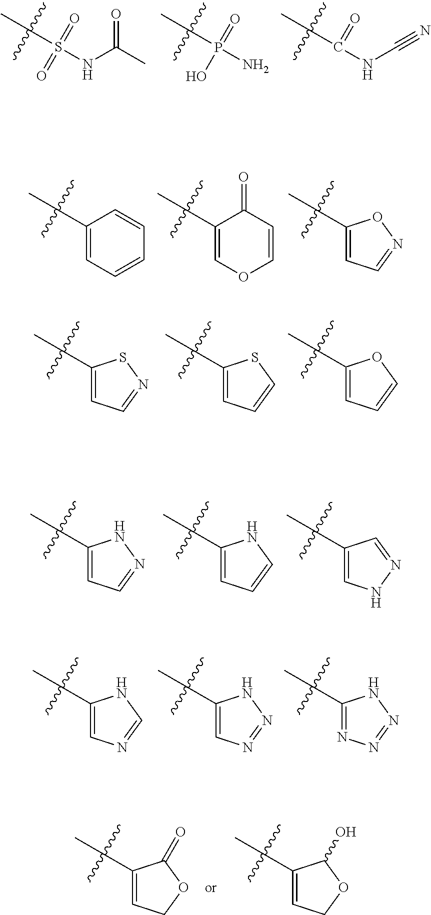

any of which may be optionally substituted with one or more groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy;
X is hydrogen, hydroxy, fluoro, chloro, bromo, cyano, nitro, C₁-C₆ alkyl, C₂-C₆ alkenyl, C₂-C₆ alkynyl, C₁-C₆ alkoxy, trifluoromethyl, or trifluoromethoxy;
and
R₃₀ is hydrogen, halogen, hydroxy, amino, hydroxyamino, nitro, cyano, —SR₅, —S(O)ₚR₅, —S(O)ₚNH₂, —S(O)ₚNHR₅, —S(O)ₚN(R₅)₂, —Si(R₅₁)₃, C₁-C₄ alkyl, C₂-C₄ alkenyl, C₂-C₄ alkynyl, C₁-C₄ alkoxy, (C₁-C₄)alkylthio, halo(C₁-C₄)alkyl, halo(C₁-C₄)alkoxy, halo(C₁-C₄)alkylthio, or mono- or di(C₁-C₄)alkylamino, where
   R₅ is C₁-C₄ alkyl, C₂-C₄ alkenyl, or C₂-C₄alkynyl; and
   p is 1 or 2.

In another embodiment of Formula ID, the present disclosure provides compounds of the formula:

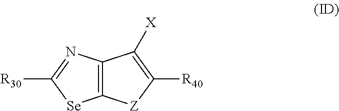

and pharmaceutically acceptable salts thereof, where
Z is —N(R_{N})— or —S—, where
   R_{N} is (i) hydrogen; (ii) C₁-C₆alkylcarbonyl optionally substituted by one or two amino groups; (iii) —S(O)ₙR₆, —S(O)ₙNH₂, —S(O)ₙNH(R₆) or —S(O)ₙN(R₆)₂, where each R₆ is independently C₁-C₆alkyl, C₂-C₆alkenyl, or C₂-C₆alkynyl, where each R₆ is optionally substituted with phenyl, and n is 1 or 2; (iv) C₁-C₆alkyl optionally substituted with one or more groups which are independently halogen or hydroxy; (v) aryl(C₁-C₂)alkyl; or (vi) heteroaryl(C₁-C₂)alkyl, where the aryl and heteroaryl groups in (v) and (vi) are optionally substituted with one or more groups which are independently halogen, hydroxy, amino, nitro, C₁-C₆alkylthio, hydroxy(C₁-C₆)alkyl, amino(C₁-C₆)alkyl, halo(C₁-C₆)alkyl, halo(C₁-C₆)alkoxy, or mono- or di (C₁-C₆) alkylamino;
R₄₀ is —COR or —R₅₀, where
   R is hydroxy, hydroxyamino, C₁-C₆alkoxy, C₁-C₆alkylcarbonyloxy, aryloxy, aryl(C₁-C₆)alkoxy, or —NR₁R₂, where R₁ and R₂ are independently (i) hydrogen, (ii) C₁-C₆alkyl, (iii) C₂-C₆alkenyl, (iv) C₂-C₆alkynyl, or (v) phenyl optionally substituted with one or more groups which are each independently halogen, hydroxy, amino, nitro, cyano, C₁-C₆alkyl, C₂-C₆alkenyl, C₂-C₆alkynyl, C₁-C₆alkoxy, C₁-C₆alkylthio, halo(C₁-C₆)alkyl, halo(C₁-C₆)alkoxy, halo(C₁-C₆)alkylthio, C₃-C₇cycloalkyl, C₅-C₇heterocycloalkyl, mono- or di(C₁-C₆)alkylamino, or carboxy; and
   R₅₀ is

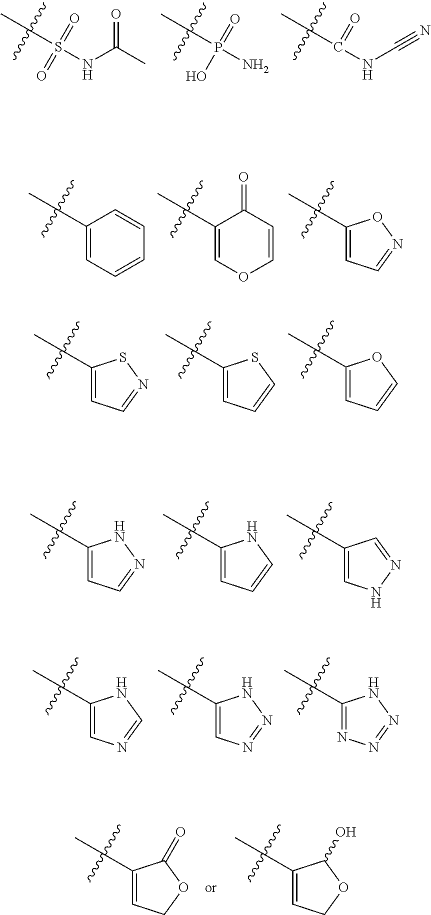

where each ring within R₅₀ is optionally substituted with one or more groups which are each independently halogen, hydroxy, nitro, C₁-C₆alkyl, C₂-C₆alkenyl, C₂-C₆alkynyl, halo(C₁-C₂)alkyl, or halo(C₁-C₂)alkoxy;
X is hydrogen, hydroxy, fluoro, chloro, cyano, nitro, C₁-C₆alkyl, C₂-C₆alkenyl, C₂-C₆alkynyl, C₁-C₆alkoxy, trifluoromethyl, or trifluoromethoxy;
and
R₃₀ is hydrogen, halogen, hydroxy, amino, hydroxyamino, nitro, cyano, —SR₅, —S(O)ₚR₅, —S(O)ₚNH₂, —S(O)ₚNHR₅, —S(O)ₚN(R₅)₂, C₁-C₄alkyl, C₂-C₄alkenyl, C₂-C₄alkynyl, C₁-C₄alkoxy, (C₁-C₄)alkylthio, halo(C₁-C₄)alkyl, halo(C₁-C₄)alkoxy, halo(C₁-C₄)alkylthio, or mono- or di(C₁-C₄)alkylamino, where
   R₅ is C₁-C₄alkyl, C₂-C₄alkenyl, or C₂-C₄alkynyl; and
   p is 1 or 2.

In a fifth aspect, the present disclosure provides compounds of one of the formulae:

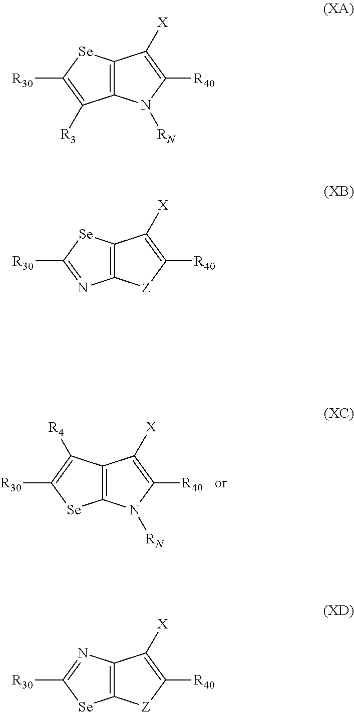

and pharmaceutically acceptable salts thereof, where
Z is —N(R_{N})— or —S—;
R_{N} is (i) hydrogen;
   (ii) C₁-C₆ alkylcarbonyl optionally substituted by one or two amino groups;
   (iii) —S(O)ₙR₆, —S(O)ₙNH₂, —S(O)ₙNH(R₆) or —S(O)ₙN(R₆)₂, where each R₆ is independently C₁-C₆ alkyl, C₂-C₆ alkenyl, or C₂-C₆ alkynyl, where each R₆ is optionally substituted with phenyl, and n is 1 or 2;
   (iv) C₁-C₆ alkyl optionally substituted with one or more groups which are independently halogen or hydroxy;
   (v) aryl(C₁-C₂)alkyl; or
   (vi) heteroaryl(C₁-C₂)alkyl,
      where the aryl and heteroaryl groups in (v) and (vi) are optionally substituted with one or more groups which are independently halogen, hydroxy, amino, nitro, C₁-C₆ alkylthio, hydroxy(C₁-C₆)alkyl, amino(C₁-C₆)alkyl, halo(C₁-C₆)alkyl, halo(C₁-C₆)alkoxy, or mono- or di (C₁-C₆)alkylamino;
R₄₀ is —COR or —R₅₀, where
   R is hydroxy, hydroxyamino, C₁-C₆ alkoxy, C₁-C₆ alkylcarbonyloxy, aryloxy, aryl(C₁-C₆)alkoxy, or —NR₁R₂, where R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or more groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆) alkyl, halo(C₁-C₆) alkoxy, halo(C₁-C₆)alkylthio, C₃-C₇ cycloalkyl, C₅-C₇ heterocycloalkyl, mono- or di (C₁-C₆)alkylamino, or carboxy; and
   R₅₀ is

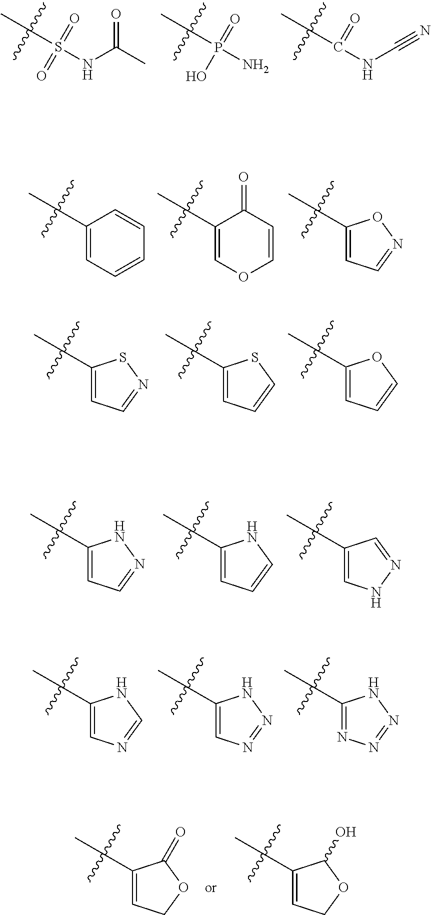

any of which may be optionally substituted with one or more groups which are each independently halogen, hydroxy, nitro, C₁-C₆ alkyl, C₂-C₆ alkenyl, C₂-C₆ alkynyl, halo(C₁-C₂) alkyl, or halo(C₁-C₂) alkoxy;
X is hydrogen, hydroxy, fluoro, chloro, bromo, cyano, nitro, C₁-C₆ alkyl, C₂-C₆ alkenyl, C₂-C₆ alkynyl, C₁-C₆ alkoxy, trifluoromethyl, or trifluoromethoxy;
and
R₃, R₄, and R₃₀ are each independently R₁₇ₐ or R₁₈, where
   R₁₇ₐ is C₃-C₇cycloalkyl, aryl, heteroaryl, aryl(C₁-C₆)alkyl, heteroaryl(C₁-C₆)alkyl, or C₅-C₇ heterocycloalkyl, where each cycloalkyl, heterocycloalkyl, aryl, and heteroaryl group is optionally substituted with one or more groups which are independently halogen, hydroxy, hydroxyamino, amino, nitro, cyano, C₁-C₆ alkyl, C₂-C₆ alkenyl, C₂-C₆ alkynyl, C₁-C₆ alkoxy, C₁-C₆ alkylthio, hydroxyalkyl, halo(C₁-C₃)alkyl, halo(C₁-C₃)alkoxy, halo(C₁-C₃)alkylthio, mono- or di(C₁-C₆)alkylamino, or amino(C₁-C₆)alkyl; and
   each R₁₈ is independently hydrogen, halogen, hydroxy, amino, hydroxyamino, nitro, cyano, —SR₅, —S(O)ₚR₅, —S(O)ₚNH₂, —S(O)ₚNHR₅, —S(O)ₚN(R₅)₂, —Si(R₅₁)₃, C₁-C₁₀ alkyl, C₂-C₁₀alkenyl, C₂-C₁₀alkynyl, C₁-C₆alkoxy, (C₁-C₆)alkylthio, halo(C₁-C₆)alkyl, halo(C₁-C₆)alkoxy, halo(C₁-C₆)alkylthio, or mono- or di(C₁-C₆)alkylamino, where each R₅ is independently C₁-C₆alkyl, C₂-C₆alkenyl, or C₂-C₆alkynyl, where each R₅ is optionally substituted by phenyl, and p is 1 or 2,
and provided that
   (a) no more than one of R₃, R₄, and R₃₀ is R₁₇;
   (b) when the compound is according to formula (IC) and R^{N} is methyl, then R⁴⁰ is not unsubstituted phenyl;
   (c) when the compound is according to formula (ID) and both Z is NH and R_{N} is —C(O)OEt, then R₃₀ is not thienyl or phenyl; and
   (d) the compound is not
      (i) 6H-selenopheno[2,3-b]pyrrole-5-carboxylic acid;
      (ii) ethyl 6H-selenopheno[2,3-b]pyrrole-5-carboxylate;
      (iii) 4H-selenopheno[3,2-b]pyrrole-5-carboxylic acid;
      (iv) methyl 4H-selenopheno[3,2-b]pyrrole-5-carboxylate; and
      (v) ethyl 4H-selenopheno[3,2-b]pyrrole-5-carboxylate.

In another aspect, the present disclosure provides compounds of one of the formulae:

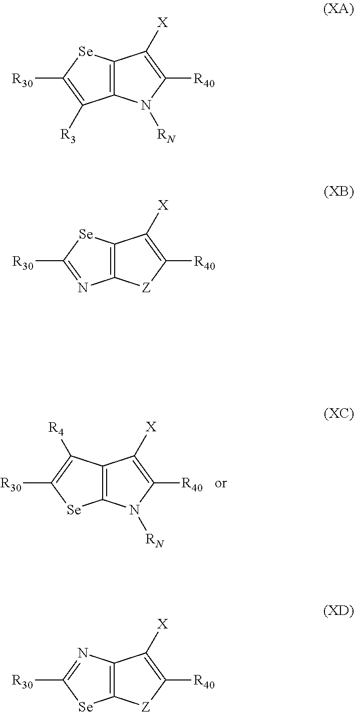

and pharmaceutically acceptable salts thereof, where
Z is —N(R_{N})— or —S—;
R_{N} is (i) hydrogen;
   (ii) C₁-C₆alkylcarbonyl optionally substituted by one or two amino groups;
   (iii) —S(O)ₙR₆, —S(O)ₙNH₂, —S(O)ₙNH(R₆), or —S(O)ₙN(R₆)₂, where each R₆ is independently C₁-C₆alkyl, C₂-C₆alkenyl, or C₂-C₆alkynyl, where each R₆ is optionally substituted with phenyl, and n is 1 or 2;
   (iv) C₁-C₆alkyl optionally substituted with one or more groups which are independently halogen or hydroxy;
   (v) aryl(C₁-C₂)alkyl; or
   (vi) heteroaryl(C₁-C₂)alkyl,
      where the aryl and heteroaryl groups in (v) and (vi) are optionally substituted with one or more groups which are independently halogen, hydroxy, amino, nitro, C₁-C₆alkylthio, hydroxy(C₁-C₆)alkyl, amino(C₁-C₆)alkyl, halo(C₁-C₆)alkyl, halo(C₁-C₆)alkoxy, or mono- or di(C₁-C₆)alkylamino;
R₄₀ is —COR or —R₅₀, where
   R is hydroxy, hydroxyamino, C₁-C₆alkoxy, C₁-C₆alkylcarbonyloxy, aryloxy, aryl(C₁-C₆)alkoxy, or —NR₁R₂, where R₁ and R₂ are independently (i) hydrogen, (ii) C₁-C₆alkyl, (iii) C₂-C₆alkenyl, (iv) C₂-C₆alkynyl, or (v) phenyl optionally substituted with one or more groups which are each independently halogen, hydroxy, amino, nitro, cyano, C₁-C₆alkyl, C₂-C₆alkenyl, C₂-C₆alkynyl, C₁-C₆alkoxy, C₁-C₆alkylthio, halo(C₁-C₆)alkyl, halo(C₁-C₆)alkoxy, halo(C₁-C₆)alkylthio, C₃-C₇cycloalkyl, C₅-C₇heterocycloalkyl, mono- or di(C₁-C₆)alkylamino, or carboxy; and
   R₅₀ is

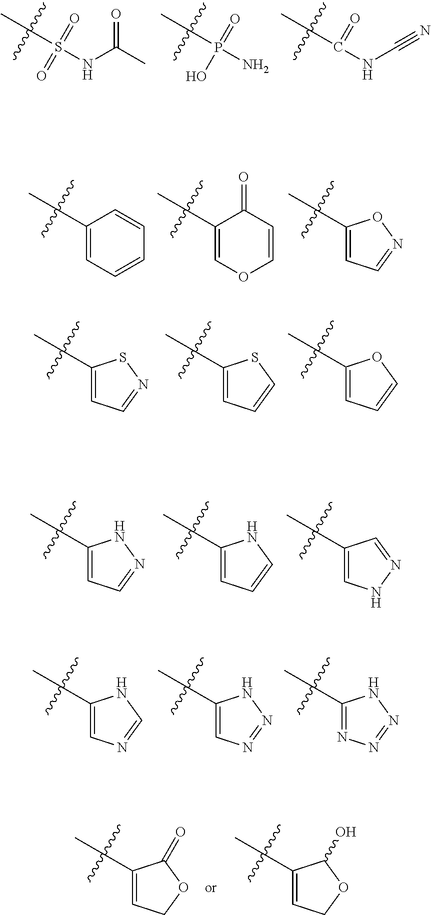

where each ring within R₅₀ is optionally substituted with one or more groups which are each independently halogen, hydroxy, nitro, C₁-C₆alkyl, C₂-C₆alkenyl, C₂-C₆alkynyl, halo(C₁-C₂)alkyl, or halo(C₁-C₂)alkoxy;
X is hydrogen, hydroxy, fluoro, chloro, cyano, nitro, C₁-C₆alkyl, C₂-C₆alkenyl, C₂-C₆alkynyl, C₁-C₆alkoxy, trifluoromethyl, or trifluoromethoxy;
and
R₃, R₄, and R₃₀ are each independently R₁₇ or R₁₈, where
   R₁₇ is C₃-C₇cycloalkyl, aryl, heteroaryl, aryl(C₁-C₆)alkyl, heteroaryl (C₁-C₆)alkyl, or C₅-C₇heterocycloalkyl, where each cycloalkyl, heterocycloalkyl, aryl, and heteroaryl group is optionally substituted with one or more groups which are independently halogen, hydroxy, hydroxyamino, amino, nitro, cyano, C₁-C₆alkyl, C₂-C₆alkenyl, C₂-C₆alkynyl, C₁-C₆alkoxy, C₁-C₆alkylthio, hydroxyalkyl, halo(C₁-C₃)alkyl, halo(C₁-C₃)alkoxy, halo(C₁-C₃)alkylthio, mono- or di(C₁-C₆)alkylamino, or amino(C₁-C₆)alkyl; and
   each R₁₈ is independently hydrogen, halogen, hydroxy, amino, hydroxyamino, nitro, cyano, —SR₅, —S(O)ₚR₅, —S(O)ₚNH₂, —S(O)ₚNHR₅, —S(O)ₚN(R₅)₂, C₁-C₁₀alkyl, C₂-C₁₀alkenyl, C₂-C₁₀alkynyl, C₁-C₆alkoxy, (C₁-C₆)alkylthio, halo(C₁-C₆)alkyl, halo(C₁-C₆)alkoxy, halo(C₁-C₆)alkylthio, or mono- or di(C₁-C₆)alkylamino, where each R₅ is independently C₁-C₆alkyl, C₂-C₆alkenyl, or C₂-C₆alkynyl, where each R₅ is optionally substituted by phenyl, and p is 1 or 2,
and provided that
   (e) no more than one of R₃, R₄, and R₃₀ is R₁₇;
   (f) when the compound is according to formula (IC) and R^{N} is methyl, then R⁴⁰ is not unsubstituted phenyl;
   (g) when the compound is according to formula (ID) and both Z is NH and R_{N} is —C(O)OEt, then R₃₀ is not thienyl or phenyl; and
   (h) the compound is not
      (i) 6H-selenopheno[2,3-b]pyrrole-5-carboxylic acid;
      (ii) ethyl 6H-selenopheno[2,3-b]pyrrole-5-carboxylate;
      (iii) 4H-selenopheno[3,2-b]pyrrole-5-carboxylic acid;
      (iv) methyl 4H-selenopheno[3,2-b]pyrrole-5-carboxylate; and
      (v) ethyl 4H-selenopheno[3,2-b]pyrrole-5-carboxylate.

In a sixth aspect, the present disclosure provides pharmaceutical compositions comprising a therapeutically effective amount of a compound or salt according to any one of the first through fifth aspects of the present disclosure and a pharmaceutically acceptable excipient, diluent or carrier.

In a seventh aspect, the present disclosure provides kits for preventing and/or treating a neurological or psychiatric disorder comprising one or more containers, where each container comprises (a) a therapeutically effective amount of a compound according to any one of the first through fifth aspects of the present disclosure, and optionally, a therapeutically effective amount of an agent useful in the prevention and/or treatment of a neurological or psychiatric disorder; or (b) a therapeutically effective amount of the composition according to the sixth aspect of the present disclosure.

In an eighth aspect, the present disclosure provides methods of preventing and/or treating a neurological or psychiatric disorder comprising administering to a patient in need thereof a therapeutically effective amount of a compound according to any one of the first through fifth aspects of the present disclosure, or a composition according to the sixth aspect of the present disclosure.

The present disclosure further provides intermediates for synthesizing compounds of the present disclosure as well as synthetic routes for preparing such compounds.

Certain compounds of the present disclosure inhibit the activity of D-aspartate oxidase (DDO), an enzyme that oxidizes D-Asp, D-Glu, D-Asn, D-Gln, D-Asp-dimethyl-ester and N-methyl-D-Asp. Methods to assay the DDO inhibitory activity of compounds are described in United States Patent Publication, US 20030166554.

### DETAILED DESCRIPTION

In one embodiment of the first aspect, the present disclosure provides compounds of formula (IA), where R₃ is hydrogen, halogen, hydroxy, hydroxyamino, amino, nitro, cyano, C₁-C₁₀ alkyl, C₂-C₁₀ alkenyl, C₂-C₁₀ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, benzyl, phenyl, where each C₃-C₇ cycloalkyl, benzyl, and phenyl is optionally substituted on the ring independently with one or two of halogen, hydroxy, hydroxyamino, nitro, cyano, C₁-C₂ alkyl, C₁-C₆ alkoxy, or halo(C₁-C₂)alkyl; such compounds are designated Formula IA-1.

In another embodiment of formula (IA), R₃ is hydrogen, fluoro, chloro, bromo, hydroxyamino, nitro, cyano, C₁-C₆ alkyl, C₁-C₆ alkoxy, C₁-C₆ alkylthio, halo(C₁-C₃)alkyl, halo(C₁-C₃)alkoxy, or halo(C₁-C₃)alkylthio; such compounds are designated Formula IA-2.

In another embodiment of formula (IA), R₃ is hydrogen, halogen, hydroxy, nitro, cyano, C₁-C₆ alkyl, C₁-C₆ alkoxy, halo(C₁-C₆)alkyl, halo(C₁-C₆)alkoxy, halo(C₁-C₆)alkylthio, or C₃-C₇ cycloalkyl; such compounds are designated Formula IA-3.

In another embodiment of formula (IA-3), R₃ is hydroxy; such compounds are designated Formula IA-4.

In another embodiment of formula (IA-3), R₃ is hydrogen; such compounds are designated Formula IA-5.

In another embodiment of formula (IA-3), R₃ is cyano; such compounds are designated Formula IA-6.

In another embodiment of formula (IA-3), R₃ is halogen; such compounds are designated Formula IA-7.

In another embodiment of formula (IA-7), R₃ is fluoro; such compounds are designated Formula IA-8.

In another embodiment of formula (IA-7), R₃ is chloro; such compounds are designated Formula IA-9.

In another embodiment of formula (IA-3), R₃ is C₁-C₆ alkyl; such compounds are designated Formula IA-10.

In another embodiment of formula (IA-10), R₃ is C₁-C₂ alkyl; such compounds are designated Formula IA-11.

In another embodiment of formula (IA-11), R₃ is methyl; such compounds are designated Formula IA-12.

In another embodiment of formula (IA-11), R₃ is ethyl; such compounds are designated Formula IA-13.

In another embodiment of formula (IA-3), R₃ is halo(C₁-C₂)alkyl; such compounds are designated Formula IA-14.

In another embodiment of formula (IA-14), R₃ is trifluoromethyl; such compounds are designated Formula IA-15.

In another embodiment of formula (IA-3), R₃ is halo(C₁-C₂)alkoxy; such compounds are designated Formula IA-16.

In another embodiment of formula (IA-16), R₃ is trifluoromethoxy; such compounds are designated Formula IA-17.

In another embodiment of formula (IA-3), R₃ is halo(C₁-C₂)alkylthio; such compounds are designated Formula IA-18.

In another embodiment of formula (IA-18), R₃ is trifluoromethylthio; such compounds are designated Formula IA-19.

In another embodiment of formula (IA-3), R₃ is nitro; such compounds are designated Formula IA-20.

In another embodiment of formula (IA-3), R₃ is C₃-C₇ cycloalkyl; such compounds are designated Formula IA-21.

In an embodiment of any one of Formulae (IA) and (IA-1-IA-21), R₃₀ is hydrogen, halogen, hydroxyamino, amino, nitro, cyano, C₁-C₄ alkyl, C₁-C₄ alkoxy, C₁-C₄ alkylthio, halo(C₁-C₄)alkyl, halo(C₁-C₄)alkoxy, or halo(C₁-C₄)alkylthio; such compounds are designated Formula IA-22.

In another embodiment of any one of Formulae (IA) and (IA-1-IA-21), R₃₀ is hydrogen, halogen, hydroxy, amino, nitro, C₁-C₃ alkoxy, or mono- or di(C₁-C₃)alkylamino; such compounds are designated Formula IA-23.

In an embodiment of Formula (IA-22), R₃₀ is hydrogen; such compounds are designated Formula IA-24.

In an embodiment of Formula (IA-22), R₃₀ is halogen; such compounds are designated Formula IA-25.

In an embodiment of Formula (IA-22), R₃₀ is methyl; such compounds are designated Formula IA-26.

In an embodiment of Formula (IA-22), R₃₀ is ethyl; such compounds are designated Formula IA-27.

In an embodiment of Formula (IA-22), R₃₀ is C₁-C₂ alkoxy; such compounds are designated Formula IA-28.

In an embodiment of Formula (IA-28), R₃₀ is methoxy; such compounds are designated Formula IA-29.

In an embodiment of Formula (IA-22), R₃₀ is halo(C₁-C₂)alkoxy; such compounds are designated Formula IA-30.

In an embodiment of Formula (IA-22), R₃₀ is halo(C₁-C₂)alkylthio; such compounds are designated Formula IA-31.

In an embodiment of Formula (IA-31), R₃₀ is trifluoromethylthio; such compounds are designated Formula IA-32.

In an embodiment of Formula (IA-22), R₃₀ is nitro; such compounds are designated Formula IA-33.

In an embodiment of Formula (IA-22), R₃₀ is amino; such compounds are designated Formula IA-34.

In an embodiment of Formula (IA-22), R₃₀ is cyano; such compounds are designated Formula IA-35.

In another embodiment of Formula (IA), R₃ and R₃₀ are independently hydrogen, fluoro, chloro, bromo, hydroxyamino, nitro, cyano, C₁-C₄ alkyl, C₁-C₄ alkoxy, C₁-C₄ alkylthio, halo(C₁-C₄)alkyl, halo(C₁-C₄)alkoxy, or halo(C₁-C₄)alkylthio; such compounds are designated Formula IA-36.

In another embodiment of Formula (IA), R₃₀ is hydrogen, halogen, hydroxy, amino, nitro, C₁-C₃ alkoxy, or mono- or di(C₁-C₃)alkylamino; and R₃ is hydrogen, halogen, hydroxy, hydroxyamino, amino, nitro, cyano, C₁-C₁₀ alkyl, C₂-C₁₀ alkenyl, C₂-C₁₀ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, benzyl, phenyl, where each C₃-C₇ cycloalkyl, benzyl, and phenyl is optionally substituted on the ring independently with one or two of halogen, hydroxy, hydroxyamino, nitro, cyano, C₁-C₂ alkyl, C₁-C₆ alkoxy, or halo(C₁-C₂)alkyl; such compounds are designated Formula IA-37.

In an embodiment of any one of Formulae (IA) and (IA-1-IA-37), R₄₀ is —COR; such compounds are designated Formula IA-38.

In an embodiment of Formula (IA-38), R is hydroxy, hydroxyamino, C₁-C₆ alkoxy, C₁-C₆ alkylcarbonyloxy, aryloxy, aryl(C₁-C₆)alkoxy, or —NR₁R₂, where R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or two groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆) alkyl, halo(C₁-C₆) alkoxy, halo(C₁-C₆) alkylthio, C₃-C₇ cycloalkyl, C₅-C₇ heterocycloalkyl, mono- or di(C₁-C₆)alkylamino, or carboxy; such compounds are designated Formula IA-39.

In an embodiment of Formula (IA-38), R is hydroxy, hydroxyamino, C₁-C₂ alkoxy, or —NR₁R₂ where R₁ and R₂ are independently hydrogen or C₁-C₂ alkyl; such compounds are designated Formula IA-40.

In an embodiment of Formula (IA-40), R is hydroxy; such compounds are designated Formula IA-41.

In an embodiment of Formula IA-41), the compound is a pharmaceutically acceptable salt thereof; such salts are designated Formula IA-42.

In an embodiment of Formula (IA-42), the salt is a calcium, d-serine (monosodium), potassium, tetramethylammonium, tris, ammonium, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, magnesium, meglumine, morpholineethanol, piperazine, pyridine, sodium, trolamine, or zinc salt; such salts are designated Formula IA-43.

In another embodiment of Formula (IA-42), the salt is a d-serine (monosodium), tris, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, meglumine, morpholineethanol, piperazine, pyridine, or trolamine salt; such salts are designated Formula IA-44.

In another embodiment of Formula (IA-42), the salt is a calcium, potassium, tetramethylammonium, ammonium, magnesium, or sodium salt; such salts are designated Formula IA-45.

In an embodiment of Formula (IA-40), R is hydroxyamino; such compounds are designated Formula IA-46.

In an embodiment of any one of Formulae (IA) and (IA-1-IA-37), R₄₀ is R₅₀; such compounds are designated Formula IA-47.

In an embodiment of Formula (IA-47), R₅₀ is selected from

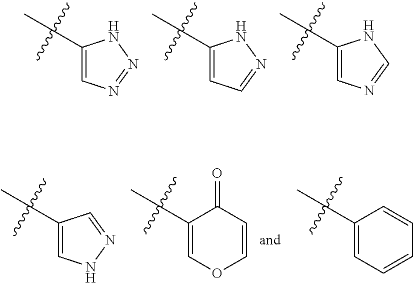

any of which may be optionally substituted with one or two groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy; such compounds are designated Formula IA-48. In an embodiment of Formula (IA-47), R₅₀ is selected from

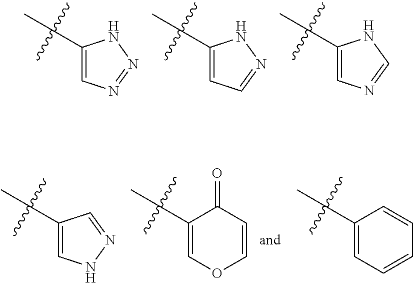

any of which may be optionally and independently substituted one or two of halogen or hydroxy; such compounds are designated Formula IA-49.

In an embodiment of Formula (IA-47), R₅₀ is selected from

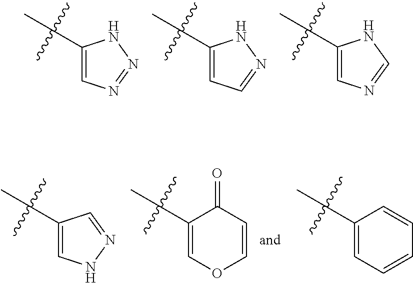

any of which may be optionally and independently substituted with one halogen or hydroxy; such compounds are designated Formula IA-50.

In an embodiment of Formula (IA-47), R₅₀ is selected from

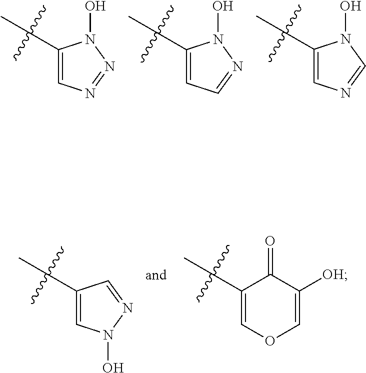

such compounds are designated Formula IA-51.

In an embodiment of Formula (IA-47), R₅₀ is selected from

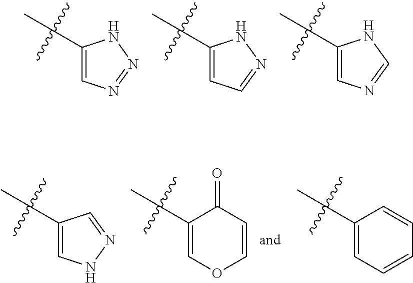

any of which may be optionally independently substituted one halogen and one hydroxy; such compounds are designated Formula IA-52.

In an embodiment of Formula (IA-49), R₅₀ is

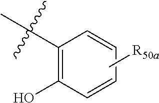

where R₅₀ₐ is hydrogen or halogen; such compounds are designated Formula IA-53.

In an embodiment of Formula (IA-53), R₅₀ₐ is hydrogen; such compounds are designated Formula IA-54.

In an embodiment of Formula (IA-53), R₅₀ₐ is halogen; such compounds are designated Formula IA-55.

In an embodiment of Formula (IA-55), R₅₀ₐ is fluoro; such compounds are designated Formula IA-56.

In an embodiment of Formula (IA-55), R₅₀ₐ is chloro; such compounds are designated Formula IA-57.

In an embodiment of any one of Formulae (IA) and (IA-1-IA-57), X is hydrogen, cyano, fluoro, chloro, trifluoromethyl, or C₁-C₆ alkyl; such compounds are designated Formula IA-58.

In an embodiment of any one of Formulae (IA) and (IA-1-IA-57), X is cyano, fluoro, chloro, trifluoromethyl, or C₁-C₆ alkyl; such compounds are designated Formula IA-59.

In an embodiment of any one of Formulae (IA) and (IA-1-IA-57), X is cyano, fluoro, chloro, trifluoromethyl, or C₁-C₃ alkyl; such compounds are designated Formula IA-60.

In an embodiment of Formula (IA-58), X is hydrogen; such compounds are designated Formula IA-61.

In an embodiment of Formula (IA-58), X is cyano; such compounds are designated Formula IA-62.

In an embodiment of Formula (IA-58), X is fluoro; such compounds are designated Formula IA-63.

In an embodiment of Formula (IA-58), X is chloro; such compounds are designated Formula IA-64.

In an embodiment of Formula (IA-58), X is trifluoromethyl; such compounds are designated Formula IA-65.

In an embodiment of Formula (IA-58), X is C₁-C₆ alkyl; such compounds are designated Formula IA-66.

In an embodiment of Formula (IA-66), X is C₁-C₃ alkyl; such compounds are designated Formula IA-67.

In an embodiment of Formula (IA-67), X is methyl; such compounds are designated Formula IA-68.

In an embodiment of Formula (IA-67), X is ethyl; such compounds are designated Formula IA-69.

In an embodiment of any one of Formulae (IA) and (IA-1-IA-69), the compound is a pharmaceutically acceptable salt; such compounds are designated Formula IA-70.

In an embodiment of Formula (IA-70), the salt is a calcium, d-serine (monosodium), potassium, tetramethylammonium, tris, ammonium, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, magnesium, meglumine, morpholineethanol, piperazine, pyridine, sodium, trolamine, lithium, or zinc salt; such salts are designated Formula IA-71.

In an embodiment of Formula (IA-70), the salt is a d-serine (monosodium), tris, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, meglumine, morpholineethanol, piperazine, pyridine, or trolamine salt; such salts are designated Formula IA-72.

In an embodiment of Formula (IA-70), the salt is a calcium, potassium, tetramethylammonium, ammonium, magnesium, lithium, or sodium salt; such salts are designated Formula IA-73.

In an embodiment of Formula (IA-70), the salt is a potassium, sodium, or lithium salt; such salts are designated Formula IA-74.

In an embodiment of the second aspect, the disclosure provides compounds of formula (IB), where Z is —N(R_{N})—; such compounds are designated formula IB-1.

In one embodiment of formulae (IB) and (IB-1), R_{N} is hydrogen, C₁-C₂ alkylcarbonyl, or C₁-C₆ alkyl; such compounds are designated formula IB-2.

In another embodiment of formulae (IB) and (IB-1), R_{N} is hydrogen; such compounds are designated formula IB-3.

In another embodiment of the second aspect, the disclosure provides compounds of formula (IB), where Z is —S—; such compounds are designated formula IB-4.

In one embodiment of any of formulae (IB) and (IB-1-IB-4), R₄₀ is —COR—; such compounds are designated formula IB-5.

In an embodiment of Formula (IB-5), R is hydroxy, hydroxyamino, C₁-C₆ alkoxy, C₁-C₆ alkylcarbonyloxy, aryloxy, aryl(C₁-C₆)alkoxy, or —NR₁R₂, where R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or two groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, C₅-C₇ heterocycloalkyl, mono- or di(C₁-C₆)alkylamino, or carboxy; such compounds are designated Formula IB-6.

In an embodiment of formula (IB-5), R is hydroxy, hydroxyamino, C₁-C₂ alkoxy, or —NR₁R₂ where R₁ and R₂ are independently hydrogen or C₁-C₂ alkyl; such compounds are designated formula IB-7.

In another embodiment of formula (IB-7), R is hydroxy; such compounds are designated formula IB-8.

In an embodiment of formula (IB-8), the compound is a pharmaceutically acceptable salt thereof; such salts are designated formula IB-9.

In an embodiment of formula (IB-9), the salt is a calcium, d-serine (monosodium), potassium, tetramethylammonium, tris, ammonium, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, magnesium, meglumine, morpholineethanol, piperazine, pyridine, sodium, trolamine, lithium, or zinc salt; such salts are designated formula IB-10.

In another embodiment of formula (IB-9), the salt is a d-serine (monosodium), tris, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, meglumine, morpholineethanol, piperazine, pyridine, or trolamine salt; such salts are designated formula IB-11.

In another embodiment of formula (IB-9), the salt is a calcium, potassium, tetramethylammonium, ammonium, magnesium, lithium, or sodium salt; such salts are designated formula IB-12.

In another embodiment of formula (IB-9), the salt is a potassium, sodium, or lithium salt; such salts are designated formula IB-13.

In another embodiment of formula (IB-7), R is hydroxyamino; such compounds are designated formula IB-14.

In one embodiment of any of formulae (IB) and (IB-1-IB-4), R₄₀ is R₅₀; such compounds are designated formula IB-15.

In an embodiment of Formula (IB-15), R₅₀ is selected from

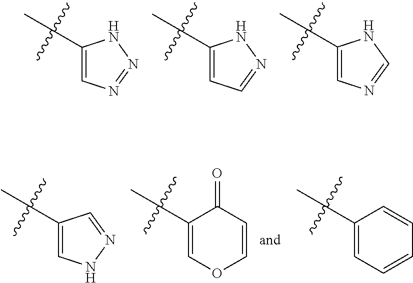

any of which may be optionally substituted with one or two groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy; such compounds are designated Formula IB-16.

In an embodiment of Formula (IB-15), R₅₀ is selected from

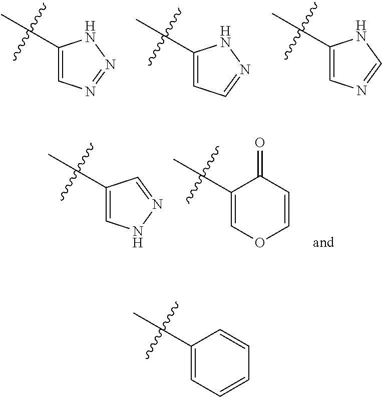

any of which may be optionally independently substituted with one or two of halogen or hydroxy; such compounds are designated Formula IB-17.

In an embodiment of Formula (IB-15), R₅₀ is selected from

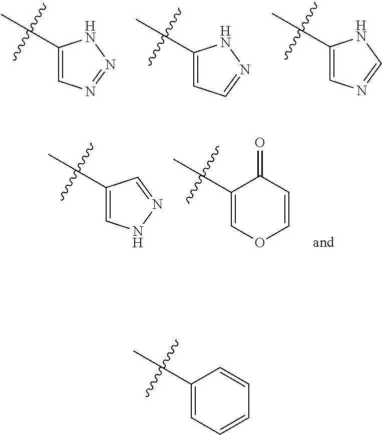

any of which may be optionally independently substituted with one halogen or hydroxy; such compounds are designated Formula IB-18.

In an embodiment of Formula (IB-15), where R₅₀ is selected from

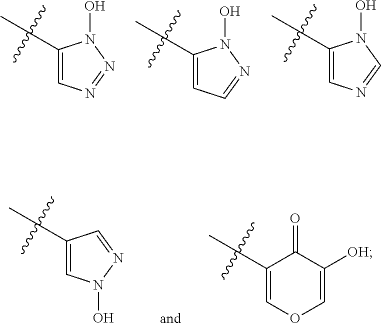

such compounds are designated Formula IB-19.

In an embodiment of Formula (IB-15), R₅₀ is selected from

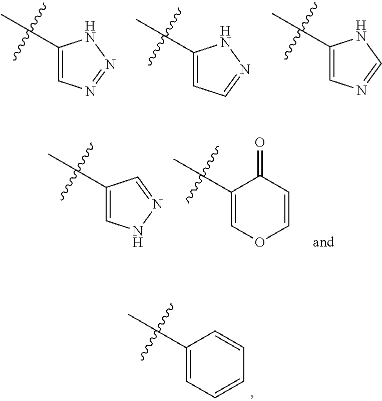

any of which may be optionally independently substituted with one halogen and one hydroxy; such compounds are designated Formula IB-20.

In an embodiment of Formula (IB-17), R₅₀ is

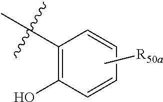

where R₅₀ₐ is hydrogen or halogen; such compounds are designated Formula IB-21.

In an embodiment of Formula (IB-21), R₅₀ₐ is hydrogen; such compounds are designated Formula IB-22.

In an embodiment of Formula (IB-21), R₅₀ₐ is halogen; such compounds are designated Formula IB-23.

In an embodiment of Formula (IB-23), R₅₀ₐ is fluoro; such compounds are designated Formula IB-24.

In an embodiment of Formula (IB-23), R₅₀ₐ is chloro; such compounds are designated Formula IB-25.

In one embodiment of any of formulae (IB) and (IB-1-IB-25), X is hydrogen, cyano, fluoro, chloro, trifluoromethyl, or C₁-C₆ alkyl; such compounds are designated Formula IB-26.

In one embodiment of any of formulae (IB) and (IB-1-IB-25), X is cyano, fluoro, chloro, trifluoromethyl, or C₁-C₆ alkyl; such compounds are designated Formula IB-27.

In one embodiment of any of formulae (IB) and (IB-1-IB-25), X is cyano, fluoro, chloro, trifluoromethyl, or C₁-C₃ alkyl; such compounds are designated Formula IB-28.

In an embodiment of Formula (IB-26), X is hydrogen; such compounds are designated Formula IB-29.

In an embodiment of Formula (IB-26), X is cyano; such compounds are designated Formula IB-30.

In an embodiment of Formula (IB-26), X is fluoro; such compounds are designated Formula IB-31.

In an embodiment of Formula (IB-26), X is chloro; such compounds are designated Formula IB-32.

In an embodiment of Formula (IB-26), X is trifluoromethyl; such compounds are designated Formula IB-33.

In an embodiment of Formula (IB-26), X is C₁-C₆ alkyl; such compounds are designated Formula IB-34.

In an embodiment of Formula (IB-34), X is methyl; such compounds are designated Formula IB-35.

In an embodiment of Formula (IB-34), X is ethyl; such compounds are designated Formula IB-36.

In one embodiment of any of formulae (IB) and (IB-1-IB-36), R₃₀ is hydrogen, halogen, hydroxyamino, amino, nitro, cyano, C₁-C₄ alkyl, C₁-C₄ alkoxy, C₁-C₄ alkylthio, halo(C₁-C₄)alkyl, halo(C₁-C₄)alkoxy, or halo(C₁-C₄)alkylthio; such compounds are designated Formula IB-37.

In another embodiment of any one of Formulae (IB) and (IB-1-IB-36), R₃₀ is hydrogen, halogen, hydroxy, amino, nitro, C₁-C₃ alkoxy, or mono- or di (C₁-C₃)alkylamino; such compounds are designated Formula IB-38.

In an embodiment of Formula (IB-37), R₃₀ is hydrogen; such compounds are designated Formula IB-39.

In an embodiment of Formula (IB-37), R₃₀ is halogen; such compounds are designated Formula IB-40.

In an embodiment of Formula (IB-37), R₃₀ is methyl; such compounds are designated Formula IB-41.

In an embodiment of Formula (IB-37), R₃₀ is ethyl; such compounds are designated Formula IB-42.

In an embodiment of Formula (IB-37), R₃₀ is C₁-C₂ alkoxy; such compounds are designated Formula IB-43.

In an embodiment of Formula (IB-43), R₃₀ is methoxy; such compounds are designated Formula IB-44.

In an embodiment of Formula (IB-37), R₃₀ is halo(C₁-C₂)alkoxy; such compounds are designated Formula IB-45.

In an embodiment of Formula (IB-37), R₃₀ is halo(C₁-C₂)alkylthio; such compounds are designated Formula IB-46.

In an embodiment of Formula (IB-46), R₃₀ is trifluoromethylthio; such compounds are designated Formula IB-47.

In an embodiment of Formula (IB-37), R₃₀ is nitro; such compounds are designated Formula IB-48.

In an embodiment of Formula (IB-37), R₃₀ is amino; such compounds are designated Formula IB-49.

In an embodiment of Formula (IB-37), R₃₀ is cyano; such compounds are designated Formula IB-50.

In another embodiment of any one of Formulae (IB) and (IB-1-IB-50), the compound is a pharmaceutically acceptable salt thereof; such salts are designated Formula IB-51.

In an embodiment of Formula (IB-51), the salt is a calcium, d-serine (monosodium), potassium, tetramethylammonium, tris, ammonium, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, magnesium, meglumine, morpholineethanol, piperazine, pyridine, sodium, trolamine, lithium, or zinc salt; such salts are designated Formula IB-52.

In an embodiment of Formula (IB-51), the salt is a d-serine (monosodium), tris, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, meglumine, morpholineethanol, piperazine, pyridine, or trolamine salt; such salts are designated Formula IB-53.

In an embodiment of Formula (IB-51), the salt is a calcium, potassium, tetramethylammonium, ammonium, magnesium, lithium, or sodium salt; such salts are designated Formula IB-54.

In another embodiment of formula (IB-51), the salt is a potassium, sodium, or lithium salt; such salts are designated Formula IB-55.

In an embodiment of the third aspect, the disclosure provides compounds of formula (IC), where R₄ is hydrogen, halogen, hydroxy, hydroxyamino, amino, nitro, cyano, C₁-C₁₀ alkyl, C₂-C₁₀ alkenyl, C₂-C₁₀ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, benzyl, phenyl, where each C₃-C₇ cycloalkyl, benzyl, and phenyl is optionally substituted on the ring independently with one or two of halogen, hydroxy, hydroxyamino, nitro, cyano, C₁-C₂ alkyl, C₁-C₆ alkoxy, or halo(C₁-C₂)alkyl; such compounds are designated formula IC-1.

In another embodiment of compounds of formula (IC), R₄ is hydrogen, halogen, hydroxy, nitro, cyano, C₁-C₆ alkyl, C₁-C₆ alkoxy, halo(C₁-C₆)alkoxy, halo(C₁-C₆)alkyl, halo(C₁-C₆)alkylthio, or C₃-C₇ cycloalkyl; such compounds are designated formula IC-2.

In another embodiment of compounds of formula (IC), R₄ is hydrogen, fluoro, chloro, bromo, hydroxyamino, nitro, cyano, C₁-C₆ alkyl, C₁-C₆ alkoxy, C₁-C₆ alkylthio, halo(C₁-C₃)alkyl, halo(C₁-C₃)alkoxy, and halo(C₁-C₃)alkylthio; such compounds are designated formula IC-3.

In an embodiment of compounds of formula (IC-2), R₄ is hydroxy; such compounds are designated formula IC-4.

In another embodiment of compounds of formula (IC-2), R₄ is hydrogen; such compounds are designated formula IC-5.

In another embodiment of compounds of formula (IC-2), R₄ is cyano; such compounds are designated formula IC-6.

In another embodiment of compounds of formula (IC-2), R₄ is halogen; such compounds are designated formula IC-7.

In another embodiment of compounds of formula (IC-7), R₄ is fluoro; such compounds are designated formula IC-8.

In another embodiment of compounds of formula (IC-7), R₄ is chloro; such compounds are designated formula IC-9.

In another embodiment of compounds of formula (IC-2), R₄ is C₁-C₆ alkyl; such compounds are designated formula IC-10.

In another embodiment of compounds of formula (IC-10), R₄ is C₁-C₂ alkyl; such compounds are designated formula IC-11.

In another embodiment of compounds of formula (IC-11), R₄ is methyl; such compounds are designated formula IC-12.

In another embodiment of compounds of formula (IC-11), R₄ is ethyl; such compounds are designated formula IC-13.

In another embodiment of compounds of formula (IC-2), R₄ is halo(C₁-C₂)alkyl; such compounds are designated formula IC-14.

In another embodiment of compounds of formula (IC-14), R₄ is trifluoromethyl; such compounds are designated formula IC-15.

In another embodiment of compounds of formula (IC-2), R₄ is halo(C₁-C₂)alkoxy; such compounds are designated formula IC-16.

In another embodiment of compounds of formula (IC-16), R₄ is trifluoromethoxy; such compounds are designated formula IC-17.

In another embodiment of compounds of formula (IC-2), R₄ is halo(C₁-C₂)alkylthio; such compounds are designated formula IC-18.

In another embodiment of compounds of formula (IC-18), R₄ is trifluoromethylthio; such compounds are designated formula IC-19.

In another embodiment of compounds of formula (IC-2), R₄ is nitro; such compounds are designated formula IC-20.

In another embodiment of compounds of formula (IC-2), R₄ is C₃-C₇ cycloalkyl; such compounds are designated formula IC-21.

In an embodiment of compounds of formula (IC) and (IC-1-IC-21), R₃₀ is hydrogen halogen, hydroxyamino, amino, nitro, cyano, C₁-C₄ alkyl, C₁-C₄ alkoxy, C₁-C₄ alkylthio, halo(C₁-C₄)alkyl, halo(C₁-C₄)alkoxy, and halo(C₁-C₄)alkylthio; such compounds are designated formula IC-22.

In another embodiment of compounds of any one of formulae (IC) and (IC-1-IC-21), R₃₀ is hydrogen, halogen, hydroxy, amino, nitro, C₁-C₃ alkoxy, or mono- or di(C₁-C₃)alkylamino; such compounds are designated formula IC-23.

In an embodiment of compounds of formula (IC-22), R₃₀ is hydrogen; such compounds are designated formula IC-24.

In another embodiment of compounds of formula (IC-22), R₃₀ is halogen; such compounds are designated formula IC-25.

In another embodiment of compounds of formula (IC-22), R₃₀ is methyl; such compounds are designated formula IC-26.

In another embodiment of compounds of formula (IC-22), R₃₀ is ethyl; such compounds are designated formula IC-27.

In another embodiment of compounds of formula (IC-22), R₃₀ is C₁-C₂ alkoxy; such compounds are designated formula IC-28.

In another embodiment of compounds of formula (IC-28), R₃₀ is methoxy; such compounds are designated formula IC-29.

In another embodiment of compounds of formula (IC-22), R₃₀ is halo(C₁-C₂)alkoxy; such compounds are designated formula IC-30.

In another embodiment of compounds of formula (IC-22), R₃₀ is halo(C₁-C₂)alkylthio; such compounds are designated formula IC-31.

In another embodiment of compounds of formula (IC-31), R₃₀ is trifluoromethylthio; such compounds are designated formula IC-32.

In another embodiment of compounds of formula (IC-22), R₃₀ is nitro; such compounds are designated formula IC-33.

In another embodiment of compounds of formula (IC-22), R₃₀ is amino; such compounds are designated formula IC-34.

In another embodiment of compounds of formula (IC-22), R₃₀ is cyano; such compounds are designated formula IC-35.

In an embodiment of formula (IC), R₃₀ and R₄ are independently hydrogen fluoro, chloro, bromo, hydroxyamino, nitro, cyano, C₁-C₄ alkyl, C₁-C₄ alkoxy, C₁-C₄ alkylthio, halo(C₁-C₄)alkyl, halo(C₁-C₄)alkoxy, and halo(C₁-C₄)alkylthio; such compounds are designated formula IC-36.

In another embodiment of formula (IC), R₃₀ is hydrogen, halogen, hydroxy, amino, nitro, C₁-C₃ alkoxy, or mono- or di(C₁-C₃)alkylamino; and R₄ is hydrogen, halogen, hydroxy, hydroxyamino, amino, nitro, cyano, C₁-C₁₀ alkyl, C₂-C₁₀ alkenyl, C₂-C₁₀ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, benzyl, phenyl, where each C₃-C₇ cycloalkyl, benzyl, and phenyl is optionally substituted on the ring independently with one or two of halogen, hydroxy, hydroxyamino, nitro, cyano, C₁-C₂ alkyl, C₁-C₆ alkoxy, or halo(C₁-C₂)alkyl; such compounds are designated formula IC-37.

In an embodiment of compounds of any one of formulae (IC) and (IC-1-IC-37), R₄₀ is —COR; such compounds are designated formula IC-38.

In an embodiment of Formula (IC-38), R is hydroxy, hydroxyamino, C₁-C₆ alkoxy, C₁-C₆ alkylcarbonyloxy, aryloxy, aryl(C₁-C₆)alkoxy, or —NR₁R₂, where R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or two groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, C₅-C₇ heterocycloalkyl, mono- or di(C₁-C₆)alkylamino, or carboxy; such compounds are designated Formula IC-39.

In an embodiment of formula (IC-38), R is hydroxy, hydroxyamino, C₁-C₂ alkoxy, or —NR₁R₂ where R₁ and R₂ are independently hydrogen or C₁-C₂ alkyl; such compounds are designated formula IC-40.

In another embodiment of formula (IC-40), R is hydroxy; such compounds are designated formula IC-41.

In another embodiment of formula (IC-41), the compound is a pharmaceutically acceptable salt thereof; such salts are designated formula IC-42.

In another embodiment of formula (IC-42), the salt is a calcium, d-serine (monosodium), potassium, tetramethylammonium, tris, ammonium, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, magnesium, meglumine, morpholineethanol, piperazine, pyridine, sodium, trolamine, lithium, or zinc salt; such salts are designated formula IC-43.

In another embodiment of formula (IC-42), the salt is a d-serine (monosodium), tris, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, meglumine, morpholineethanol, piperazine, pyridine, or trolamine salt; such compounds are designated formula IC-44.

In another embodiment of formula (IC-42), the salt is a calcium, potassium, tetramethylammonium, ammonium, magnesium, lithium, or sodium salt; such salts are designated formula IC-45.

In another embodiment of formula (IC-42), the salt is a potassium, sodium, or lithium salt; such salts are designated formula IC-46.

In an embodiment of formula (IC-40), R is hydroxyamino; such compounds are designated formula IC-47.

In another embodiment of compounds of any one of formula (IC) and (IC-1-IC-37), R₄₀ is R₅₀; such compounds are designated formula IC-48.

In an embodiment of Formula (IC-48), R₅₀ is selected from

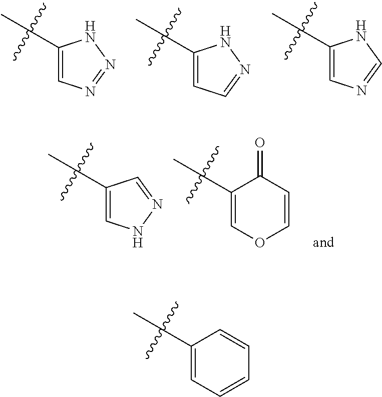

any of which may be optionally substituted with one or two groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy; such compounds are designated formula IC-49.

In another embodiment of Formula (IC-48), R₅₀ is selected from

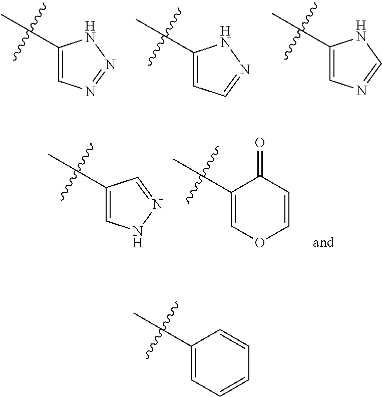

any of which may be optionally and independently substituted with one or two of halogen or hydroxy; such compounds are designated formula IC-50.

In another embodiment of Formula (IC-48), R₅₀ is selected from

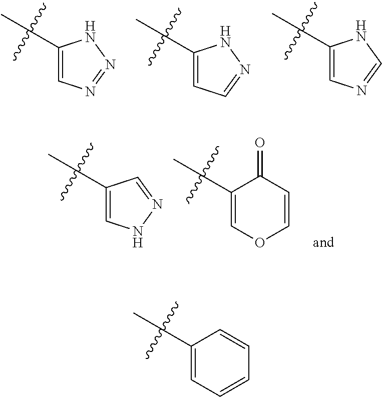

any of which may be optionally and independently substituted with one halogen or hydroxy; such compounds are designated formula IC-51.

In another embodiment of Formula (IC-48), R₅₀ is selected from

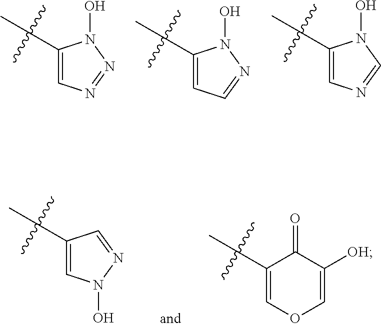

such compounds are designated formula IC-52.

In another embodiment of Formula (IC-48), R₅₀ is selected from

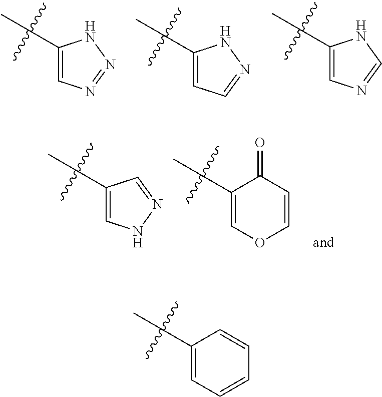

where each aryl or heteroaryl ring is optionally independently substituted one halogen and one hydroxy; such compounds are designated formula IC-53.

In another embodiment of Formula (IC-50), R₅₀ is

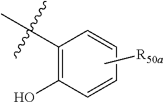

where R₅₀ₐ is hydrogen or halogen; such compounds are designated formula IC-54.

In an embodiment of Formula (IC-54), R₅₀ₐ is hydrogen; such compounds are designated formula IC-55.

In an embodiment of Formula (IC-54), R₅₀ₐ is halogen; such compounds are designated formula IC-56.

In an embodiment of Formula (IC-56), R₅₀ₐ is fluoro; such compounds are designated formula IC-57.

In an embodiment of Formula (IC-56), R₅₀ₐ is chloro; such compounds are designated formula IC-58.

In an embodiment of compounds of any one of formula (IC) and (IC-1-IC-58), X is hydrogen, cyano, fluoro, chloro, trifluoromethyl, or C₁-C₆ alkyl; such compounds are designated formula IC-59.

In another embodiment of compounds of any one of formula (IC) and (IC-1-IC-58), X is cyano, fluoro, chloro, trifluoromethyl, or C₁-C₆ alkyl; such compounds are designated formula IC-60.

In another embodiment of compounds of any one of formula (IC) and (IC-1-IC-58), X is cyano, fluoro, chloro, bromo, trifluoromethyl, or C₁-C₃ alkyl; such compounds are designated formula IC-61.

In an embodiment of formula (IC-59), X is hydrogen; such compounds are designated formula IC-62.

In another embodiment of formula (IC-59), X is cyano; such compounds are designated formula IC-63.

In another embodiment of formula (IC-59), X is fluoro; such compounds are designated formula IC-64.

In another embodiment of formula (IC-59), X is chloro; such compounds are designated formula IC-65.

In another embodiment of formula (IC-59), X is trifluoromethyl; such compounds are designated formula IC-66.

In another embodiment of formula (IC-59), X is C₁-C₆ alkyl; such compounds are designated formula IC-67.

In another embodiment of formula (IC-67), X is methyl; such compounds are designated formula IC-68.

In another embodiment of formula (IC-67), X is ethyl; such compounds are designated formula IC-69.

In an embodiment of compounds of any one of formula (IC) and (IC-1-IC-69), the compound is a pharmaceutically acceptable salt thereof; such salts are designated formula IC-70.

In an embodiment of formula (IC-70), the salt is a calcium, d-serine (monosodium), potassium, tetramethylammonium, tris, ammonium, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, magnesium, meglumine, morpholineethanol, piperazine, pyridine, sodium, trolamine, lithium, or zinc salt; such salts are designated formula IC-71.

In an embodiment of formula (IC-70), the salt is a d-serine (monosodium), tris, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, meglumine, morpholineethanol, piperazine, pyridine, or trolamine salt; such salts are designated formula IC-72.

In an embodiment of formula (IC-70), the salt is a calcium, potassium, tetramethylammonium, ammonium, magnesium, lithium, or sodium salt; such salts are designated formula IC-73.

In an embodiment of formula (IC-70), the salt is a potassium, sodium, or lithium salt; such salts are designated formula IC-74.

In an embodiment of the fourth aspect, the disclosure provides compounds of formula (ID), where Z is —N(R_{N})—; such compounds are designated formula ID-1.

In an embodiment of formula (ID-1), R_{N} is hydrogen, C₁-C₂ alkylcarbonyl, or C₁-C₆ alkyl; such compounds are designated formula ID-2.

In an embodiment of formula (ID-2), R_{N} is hydrogen; such compounds are designated formula ID-3.

In another embodiment of formula (ID), where Z is —S—; such compounds are designated formula ID-4.

In an embodiment of any one of formulae (ID) and (ID-1-ID-4), R₄₀ is —COR; such compounds are designated formula ID-5.

In an embodiment of Formula (ID-5), R is hydroxy, hydroxyamino, C₁-C₆ alkoxy, C₁-C₆ alkylcarbonyloxy, aryloxy, aryl(C₁-C₆)alkoxy, or —NR₁R₂, where R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or two groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, C₅-C₇ heterocycloalkyl, mono- or di(C₁-C₆)alkylamino, or carboxy; such compounds are designated Formula ID-6.

In an embodiment of formula (ID-5), R is hydroxy, hydroxyamino, C₁-C₂ alkoxy, or —NR₁R₂ where R₁ and R₂ are independently hydrogen or C₁-C₂ alkyl; such compounds are designated formula ID-7.

In an embodiment of formula (ID-7), R is hydroxy; such compounds are designated formula ID-8.

In an embodiment of formula (ID-8), the compound is a pharmaceutically acceptable salt thereof; such salts are designated formula ID-9.

In an embodiment of formula (ID-9), the salt is a calcium, d-serine (monosodium), potassium, tetramethylammonium, tris, ammonium, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, magnesium, meglumine, morpholineethanol, piperazine, pyridine, sodium, trolamine, lithium, or zinc salt; such salts are designated formula ID-10.

In an embodiment of formula (ID-9), the salt is a d-serine (monosodium), tris, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, meglumine, morpholineethanol, piperazine, pyridine, or trolamine salt; such salts are designated formula ID-11.

In an embodiment of formula (ID-9), the salt is a calcium, potassium, tetramethylammonium, ammonium, magnesium, lithium, or sodium salt; such salts are designated formula ID-12.

In an embodiment of formula (ID-9), the salt is a potassium, sodium, or lithium salt; such salts are designated formula ID-13.

In an embodiment of formula (ID-7), R is hydroxyamino; such compounds are designated formula ID-14.

In an embodiment of any one of formulae (ID) and (ID-1-ID-4), R₄₀ is R₅₀; such compounds are designated formula ID-15.

In an embodiment of Formula (ID-15), R₅₀ is selected from

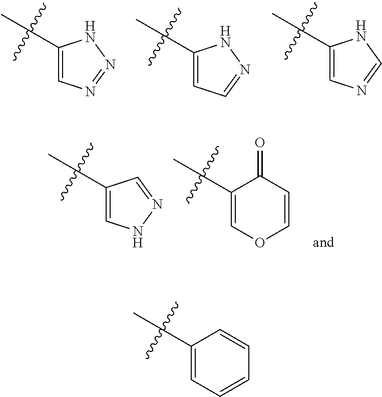

any of which may be optionally substituted with one or two groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy; such compounds are designated formula ID-16.

In an embodiment of formula (ID-15), R₅₀ is selected from

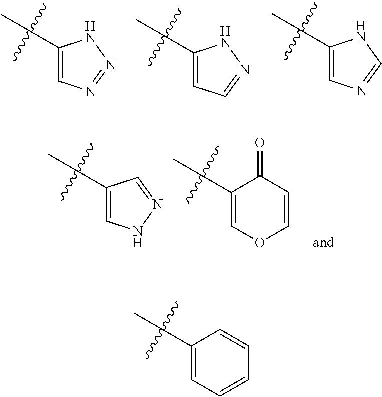

any of which may be optionally and independently substituted at a substitutable position with one or two of halogen or hydroxy; such compounds are designated formula ID-17.

In another embodiment of Formula (ID-15), R₅₀ is selected from

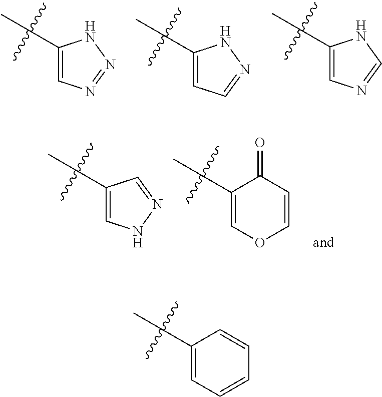

any of which may be optionally and independently substituted with one halogen or hydroxy; such compounds are designated formula ID-18.

In an embodiment of formula (ID-15), R₅₀ is selected from

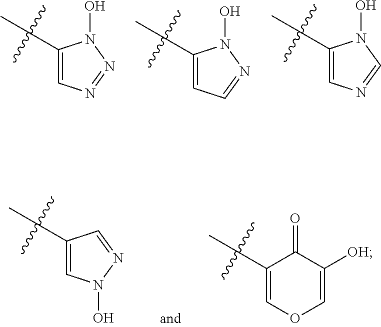

such compounds are designated formula ID-19.

In another embodiment of Formula (ID-15), R₅₀ is selected from

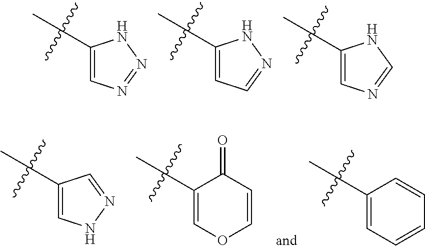

any of which may be optionally and independently substituted one halogen and one hydroxy; such compounds are designated formula ID-20.

In an embodiment of formula (ID-17), R₅₀ is

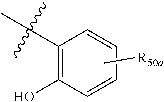

where R₅₀ₐ is hydrogen or halogen; such compounds are designated formula ID-21.

In an embodiment of formula (ID-21), R₅₀ₐ is hydrogen; such compounds are designated formula ID-22.

In an embodiment of formula (ID-21), R₅₀ₐ is halogen; such compounds are designated formula ID-23.

In an embodiment of formula (ID-23), R₅₀ₐ is fluoro; such compounds are designated formula ID-24.

In an embodiment of formula (ID-23), R₅₀ₐ is chloro; such compounds are designated formula ID-25.

In an embodiment of any one of formulae (ID) and (ID-1-ID-25), X is hydrogen, cyano, fluoro, chloro, trifluoromethyl, or C₁-C₆ alkyl; such compounds are designated formula ID-26.

In an embodiment of any one of formulae (ID) and (ID-1-ID-25), X is cyano, fluoro, chloro, trifluoromethyl, or C₁-C₆ alkyl; such compounds are designated formula ID-27.

In an embodiment of any one of formulae (ID) and (ID-1-ID-25), X is cyano, fluoro, chloro, trifluoromethyl, or C₁-C₃ alkyl; such compounds are designated formula ID-28.

In an embodiment of formula (ID-26), X is hydrogen; such compounds are designated formula ID-29.

In an embodiment of formula (ID-26), X is cyano; such compounds are designated formula ID-30.

In an embodiment of formula (ID-26), X is fluoro; such compounds are designated formula ID-31.

In an embodiment of formula (ID-26), X is chloro; such compounds are designated formula ID-32.

In an embodiment of formula (ID-26), X is trifluoromethyl; such compounds are designated formula ID-33.

In an embodiment of formula (ID-26), X is C₁-C₆ alkyl; such compounds are designated formula ID-34.

In an embodiment of formula (ID-34), X is methyl; such compounds are designated formula ID-35.

In an embodiment of formula (ID-34), X is ethyl; such compounds are designated formula ID-36.

In an embodiment of any one of formulae (ID) and (ID-1-ID-36), R₃₀ is hydrogen, halogen, hydroxyamino, amino, nitro, cyano, C₁-C₄ alkyl, C₁-C₄ alkoxy, C₁-C₄ alkylthio, halo(C₁-C₄)alkyl, halo(C₁-C₄)alkoxy, and halo(C₁-C₄)alkylthio; such compounds are designated formula ID-37.

In an embodiment of formula (ID-37), R₃₀ is hydrogen; such compounds are designated formula ID-38.

In an embodiment of formula (ID-37), R₃₀ is halogen; such compounds are designated formula ID-39.

In an embodiment of formula (ID-37), R₃₀ is methyl; such compounds are designated formula ID-40.

In an embodiment of formula (ID-37), R₃₀ is ethyl; such compounds are designated formula ID-41.

In an embodiment of formula (ID-37), R₃₀ is C₁-C₂ alkoxy; such compounds are designated formula ID-42.

In an embodiment of formula (ID-42), R₃₀ is methoxy; such compounds are designated formula ID-43.

In an embodiment of formula (ID-37), R₃₀ is halo(C₁-C₂)alkoxy; such compounds are designated formula ID-44.

In an embodiment of formula (ID-37), R₃₀ is halo(C₁-C₂)alkylthio; such compounds are designated formula ID-45.

In an embodiment of formula (ID-45), R₃₀ is trifluoromethylthio; such compounds are designated formula ID-46.

In an embodiment of formula (ID-37), R₃₀ is nitro; such compounds are designated formula ID-47.

In an embodiment of formula (ID-37), R₃₀ is amino; such compounds are designated formula ID-48.

In an embodiment of formula (ID-37), R₃₀ is cyano; such compounds are designated formula ID-49.

In an embodiment of any one of formulae (ID) and (ID-1-ID-49), the compound is a pharmaceutically acceptable salt thereof; such salts are designated formula ID-50.

In an embodiment of formula (ID-50), the salt is a calcium, d-serine (monosodium), potassium, tetramethylammonium, tris, ammonium, benethamine, benzathine, choline, clemizole, deanol, dicyclohexylamine, diethanolamine, diethylamine, diethylaminoethanol, epolamine; ethanolamine, ethylenediamine, ethylpropylammonium, hydrabamine, imidazole, l-lysine, magnesium, meglumine, morpholineethanol, piperazine, pyridine, sodium, trolamine, lithium, or zinc salt; such salts are designated formula ID-51.

In an embodiment of formula (ID-50), the salt is a d-serine (monosodium), tris, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, meglumine, morpholineethanol, piperazine, pyridine, or trolamine salt; such salts are designated formula ID-52.

In an embodiment of formula (ID-50), the salt is a calcium, potassium, tetramethylammonium, ammonium, magnesium, lithium, or sodium salt; such salts are designated formula ID-53.

In an embodiment of formula (ID-50), the salt is a potassium, sodium, or lithium salt; such salts are designated formula ID-54.

In an embodiment of the fifth aspect, the disclosure provides the compounds of formulae (XA)-(XD), where each R₁₈ is independently hydrogen, fluoro, chloro, bromo, hydroxyamino, nitro, cyano, C₁-C₆ alkyl, C₁-C₆ alkoxy, C₁-C₆ alkylthio, halo(C₁-C₃)alkyl, halo(C₁-C₃)alkoxy, and halo(C₁-C₃)alkylthio.

In another embodiment of formulae (XA)-(XD), R₃₀ is hydrogen, halogen, hydroxy, amino, nitro, C₁-C₃ alkoxy, or mono- or di(C₁-C₃)alkylamino; R₃, when present, is hydrogen, halogen, hydroxy, hydroxyamino, amino, nitro, cyano, C₁-C₁₀ alkyl, C₂-C₁₀ alkenyl, C₂-C₁₀ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, benzyl, phenyl, where each C₃-C₇ cycloalkyl, benzyl, and phenyl is optionally substituted on the ring independently with one or two of halogen, hydroxy, hydroxyamino, nitro, cyano, C₁-C₂ alkyl, C₁-C₆ alkoxy, or halo(C₁-C₂)alkyl; and R₄, when present, is hydrogen, halogen, hydroxy, hydroxyamino, amino, nitro, cyano, C₁-C₁₀ alkyl, C₂-C₁₀ alkenyl, C₂-C₁₀ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, benzyl, phenyl, where each C₃-C₇ cycloalkyl, benzyl, and phenyl is optionally substituted on the ring independently with one or two of halogen, hydroxy, hydroxyamino, nitro, cyano, C₁-C₂ alkyl, C₁-C₆ alkoxy, or halo(C₁-C₂)alkyl.

In an embodiment of the fifth aspect, the disclosure provides the compounds of formula (XA).

In an embodiment of the fifth aspect, the disclosure provides the compounds of formula (XB).

In an embodiment of the fifth aspect, the disclosure provides the compounds of formula (XC).

In an embodiment of the fifth aspect, the disclosure provides the compounds of formula (XD).

In an embodiment of formulae (XA) and (XC), either R₃ or R₄, when present, is hydrogen, halogen, hydroxy, nitro, cyano, C₁-C₆ alkyl, C₁-C₆ alkoxy, halo(C₁-C₆)alkyl, halo(C₁-C₆)alkoxy, halo(C₁-C₆)alkylthio, or C₃-C₇ cycloalkyl; such compounds are designated formulae (XA-1) and (XC-1), respectively.

In an embodiment of formulae (XA) and (XC), either R₃ or R₄, when present, is hydroxy; such compounds are designated formulae (XA-2) and (XC-2), respectively.

In another embodiment of formulae (XA) and (XC), either R₃ or R₄, when present, is hydrogen; such compounds are designated formulae (XA-3) and (XC-3), respectively.

In another embodiment of formulae (XA) and (XC), either R₃ or R₄, when present, is cyano; such compounds are designated formulae (XA-4) and (XC-4), respectively.

In another embodiment of formulae (XA) and (XC), either R₃ or R₄, when present, is halogen; such compounds are designated formulae (XA-5) and (XC-5), respectively.

In an embodiment of formulae (XA-5) and (XC-5), either R₃ or R₄, when present, is fluoro; such compounds are designated formulae (XA-6) and (XC-6), respectively.

In another embodiment of formulae (XA-5) and (XC-5), either R₃ or R₄, when present, is chloro; such compounds are designated formulae (XA-7) and (XC-7), respectively.

In another embodiment of formulae (XA) and (XC), either R₃ or R₄, when present, is C₁-C₆ alkyl; such compounds are designated formulae (XA-8) and (XC-8), respectively.

In an embodiment of formulae (XA-8) and (XC-8), either R₃ or R₄, when present, is C₁-C₂ alkyl; such compounds are designated formulae (XA-9) and (XC-9), respectively.

In an embodiment of formulae (XA-9) and (XC-9), either R₃ or R₄, when present, is methyl; such compounds are designated formulae (XA-10) and (XC-10), respectively.

In another embodiment of formulae (XA-9) and (XC-9), either R₃ or R₄, when present, is ethyl; such compounds are designated formulae (XA-11) and (XC-11), respectively.

In another embodiment of formulae (XA) and (XC), either R₃ or R₄, when present, is halo(C₁-C₂)alkyl; such compounds are designated formulae (XA-12) and (XC-12), respectively.

In an embodiment of formulae (XA-12) and (XC-12), either R₃ or R₄, when present, is trifluoromethyl; such compounds are designated formulae (XA-13) and (XC-13), respectively.

In another embodiment of formulae (XA) and (XC), either R₃ or R₄, when present, is halo(C₁-C₂)alkoxy; such compounds are designated formulae (XA-14) and (XC-14), respectively.

In an embodiment of formulae (XA-14) and (XC-14), either R₃ or R₄, when present, is trifluoromethoxy; such compounds are designated formulae (XA-15) and (XC-15), respectively.

In another embodiment formulae (XA) and (XC), either R₃ or R₄, when present, is halo(C₁-C₂)alkylthio; such compounds are designated formulae (XA-16) and (XC-16), respectively.

In an embodiment of formulae (XA-16) and (XC-16), either R₃ or R₄, when present, is trifluoromethylthio; such compounds are designated formulae (XA-17) and (XC-17), respectively.

In another embodiment of formulae (XA) and (XC), either R₃ or R₄, when present, is nitro; such compounds are designated formulae (XA-18) and (XC-18), respectively.

In another embodiment of formulae (XA) and (XC), either R₃ or R₄, when present, is C₃-C₇ cycloalkyl; such compounds are designated formulae (XA-19) and (XC-19), respectively.

In an embodiment of formulae (XB) and (XD), Z is —N(R_{N})—; such compounds are designated formulae (XB-20) and (XD-20), respectively.

In an embodiment of formulae (XB-20) and (XD-20), R_{N} is hydrogen, C₁-C₂ alkylcarbonyl, or C₁-C₆ alkyl; such compounds are designated formulae (XB-21) and (XD-21), respectively.

In another embodiment of formulae (XB-20) and (XD-20), R_{N} is hydrogen; such compounds are designated formulae (XA-22)-(XD-22), respectively.

In another embodiment of formulae (XB) and (XD), Z is —S; such compounds are designated formulae (XB-23) and (XD-23), respectively.

In an embodiment of any one of formulae (XA)-(XD), (XA-1)-(XA-19), (XB-20)-(XB-23), (XC-1)-(XC-19), and (XD-20)-(XD-23), R₄₀ is —COR; such compounds are designated formulae (XA-24)-(XD-24), respectively.

In an embodiment of any one of formulae (XA-24)-(XD-24), R is hydroxy, hydroxyamino, C₁-C₂ alkoxy, or —NR₁R₂ where R₁ and R₂ are independently hydrogen or C₁-C₂ alkyl; such compounds are designated formulae (XA-25)-(XD-25), respectively.

In an embodiment of any one of formulae (XA-25)-(XD-25), R is hydroxy; such compounds are designated formulae (XA-26)-(XD-26), respectively.

In an embodiment of any one of formulae (XA-26)-(XD-26), the compound is a pharmaceutically acceptable salt thereof; such compounds are designated formulae (XA-27)-(XD-27), respectively.

In an embodiment of any one of formulae (XA-27)-(XD-27), the compound is a calcium, d-serine (monosodium), potassium, tetramethylammonium, tris, ammonium, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, magnesium, meglumine, morpholineethanol, piperazine, pyridine, sodium, trolamine, or zinc salt; such compounds are designated formulae (XA-28)-(XD-28), respectively.

In another embodiment of any one of formulae (XA-24)-(XD-24), R is hydroxyamino; such compounds are designated formulae (XA-29)-(XD-29), respectively.

In another embodiment of any one of formulae (XA)-(XD), (XA-1)-(XA-19), (XB-20)-(XB-23), (XC-1)-(XC-19), and (XD-20)-(XD-23), R₄₀ is R₅₀; such compounds are designated formulae (XA-30)-(XD-30), respectively.

In an embodiment of any one of formulae (XA-30)-(XD-30), R₅₀ is selected from

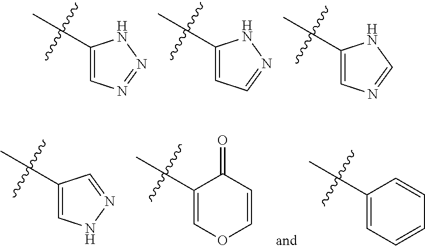

any of which may be optionally and independently substituted at a substitutable position with one or two of halogen or hydroxy; such compounds are designated formulae (XA-31)-(XD-31), respectively.

In another embodiment of any one of formulae (XA-31)-(XD-31), R₅₀ is selected from

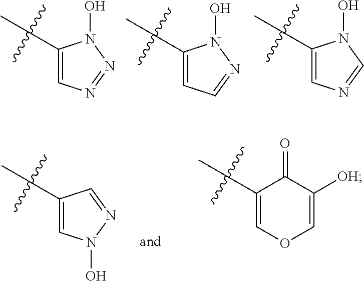

such compounds are designated formulae (XA-32)-(XD-32), respectively.

In an embodiment of any one of formulae (XA-31)-(XD-31), R₅₀ is

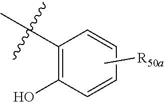

where R₅₀ₐ is hydrogen or halogen; such compounds are designated formulae (XA-33)-(XD-33), respectively.

In an embodiment of any one of formulae (XA-33)-(XD-33), R₅₀ₐ is hydrogen; such compounds are designated formulae (XA-34)-(XD-34), respectively.

In another embodiment of any one of formulae (XA-33)-(XD-33), R₅₀ₐ is halogen; such compounds are designated formulae (XA-35)-(XD-35), respectively.

In an embodiment of any one of formulae (XA-35)-(XD-35), R₅₀ₐ is fluoro; such compounds are designated formulae (XA-36)-(XD-36), respectively.

In another embodiment of any one of formulae (XA-35)-(XD-35), R₅₀ₐ is chloro; such compounds are designated formulae (XA-37)-(XD-37), respectively.

In an embodiment of any one of formulae (XA)-(XD), (XA-1)-(XA-19), (XA-24)-(XA-37), (XB-20)-(XB-37), (XC-1)-(XC-19), (XC-24)-(XC-37), and (XD-20)-(XD-37), X is hydrogen, cyano, fluoro, chloro, trifluoromethyl, or C₁-C₆ alkyl; such compounds are designated formulae (XA-38)-(XD-38), respectively.

In an embodiment of any one of formulae (XA-38)-(XD-38), X is hydrogen; such compounds are designated formulae (XA-39)-(XD-39), respectively.

In an embodiment of any one of formulae (XA-38)-(XD-38), X is cyano; such compounds are designated formulae (XA-40)-(XD-40), respectively.

In an embodiment of any one of formulae (XA-38)-(XD-38), X is fluoro; such compounds are designated formulae (XA-41)-(XD-41), respectively.

In an embodiment of any one of formulae (XA-38)-(XD-38), X is chloro; such compounds are designated formulae (XA-42)-(XD-42), respectively.

In an embodiment of any one of formulae (XA-38)-(XD-38), X is trifluoromethyl; such compounds are designated formulae (XA-43)-(XD-43), respectively.

In an embodiment of any one of formulae (XA-38)-(XD-38), X is C₁-C₆ alkyl; such compounds are designated formulae (XA-44)-(XD-44), respectively.

In an embodiment of any one of formulae (XA-44)-(XD-44), X is methyl; such compounds are designated formulae (XA-45)-(XD-45), respectively.

In another embodiment of any one of formulae (XA-44)-(XD-44), where X is ethyl; such compounds are designated formulae (XA-46)-(XD-46), respectively.

In an embodiment of any one of formulae (XA)-(XD), (XA-1)-(XA-19), (XA-24)-(XA-46), (XB-20)-(XB-46), (XC-1)-(XC-19), (XC-24)-(XC-46), and (XD-20)-(XD-46), R₃₀ is hydrogen, halogen, hydroxyamino, amino, nitro, cyano, C₁-C₆ alkyl, C₁-C₆ alkoxy, C₁-C₆ alkylthio, halo(C₁-C₃)alkyl, halo(C₁-C₃)alkoxy, and halo(C₁-C₃)alkylthio; such compounds are designated formulae (XA-47)-(XD-47), respectively.

In an embodiment of any one of formulae (XA-47)-(XD-47), R₃₀ is hydrogen; such compounds are designated formulae (XA-48)-(XD-48), respectively.

In another embodiment of any one of formulae (XA-47)-(XD-47), R₃₀ is halogen; such compounds are designated formulae (XA-49)-(XD-49), respectively.

In another embodiment of any one of formulae (XA-47)-(XD-47), R₃₀ is methyl; such compounds are designated formulae (XA-50)-(XD-50), respectively.

In another embodiment of any one of formulae (XA-47)-(XD-47), R₃₀ is ethyl; such compounds are designated formulae (XA-51)-(XD-51), respectively.

In another embodiment of any one of formulae (XA-47)-(XD-47), R₃₀ is C₁-C₂ alkoxy; such compounds are designated formulae (XA-52)-(XD-52), respectively.

In an embodiment of any one of formulae (XA-52)-(XD-52), R₃₀ is methoxy; such compounds are designated formulae (XA-53)-(XD-53), respectively.

In another embodiment of any one of formulae (XA-47)-(XD-47), R₃₀ is halo(C₁-C₂)alkoxy; such compounds are designated formulae (XA-54)-(XD-54), respectively.

In another embodiment of any one of formulae (XA-47)-(XD-47), R₃₀ is halo(C₁-C₂)alkylthio; such compounds are designated formulae (XA-55)-(XD-55), respectively.

In an embodiment of any one of formulae (XA-55)-(XD-55), R₃₀ is trifluoromethylthio; such compounds are designated formulae (XA-56)-(XD-56), respectively.

In another embodiment of any one of formulae (XA-47)-(XD-47), R₃₀ is nitro; such compounds are designated formulae (XA-57)-(XD-57), respectively.

In another embodiment of any one of formulae (XA-47)-(XD-47), R₃₀ is amino; such compounds are designated formulae (XA-58)-(XD-58), respectively.

In another embodiment of any one of formulae (XA-47)-(XD-47), R₃₀ is cyano; such compounds are designated formulae (XA-59)-(XD-59), respectively.

In an embodiment of any one of formulae (XA)-(XD), (XA-1)-(XA-19), (XA-24)-(XA-59), (XB-20)-(XB-59), (XC-1)-(XC-19), (XC-24)-(XC-59), and (XD-20)-(XD-59), the compound is a pharmaceutically acceptable salt thereof; such compounds are designated formulae (XA-60)-(XD-60), respectively.

In an embodiment of any one of formulae (XA-60)-(XD-60), the compound is a calcium, d-serine (monosodium), potassium, tetramethylammonium, tris, ammonium, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, magnesium, meglumine, morpholineethanol, piperazine, pyridine, sodium, trolamine, lithium, or zinc salt; such compounds are designated formulae (XA-61)-(XD-61), respectively.

In another embodiment of any one of formulae (XA-60)-(XD-60), the compound is a d-serine (monosodium), tris, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, l-lysine, meglumine, morpholineethanol, piperazine, pyridine, or trolamine salt; such compounds are designated formulae (XA-62)-(XD-62), respectively.

In another embodiment of any one of formulae (XA-60)-(XD-60), the compound is a calcium, potassium, tetramethylammonium, ammonium, magnesium, lithium, or sodium salt; such compounds are designated formulae (XA-63)-(XD-63), respectively.

In another embodiment of any one of formulae (XA-60)-(XD-60), the compound is a potassium, sodium, or lithium salt; such compounds are designated formulae (XA-64)-(XD-64), respectively.

In an embodiment of the sixth aspect, the present disclosure provides compositions further comprising one or more agents useful in the prevention and/or treatment of a neurological or psychiatric disorder; such compositions are designated composition 6-1.

In another embodiment of the sixth aspect, the present disclosure provides compositions according to composition 6-1, where the one or more agents are chosen from D-amino acids and derivatives thereof, anti-psychotics, and anticholinergics; such compositions are designated composition 6-2.

In another embodiment of the sixth aspect, the present disclosure provides compositions according to composition 6-2 where at least one of the one or more agents is a D-amino acid or derivative thereof; such compositions are designated composition 6-3.

In another embodiment of the sixth aspect, the present disclosure provides compositions according to composition 6-3 where the D-amino acids or derivative thereof is D-cycloserine, D-serine or a D-serine analog; such compositions are designated composition 6-4.

In another embodiment of the sixth aspect, the present disclosure provides compositions according to composition 6-4, where the D-amino acid or derivative thereof is D-serine; such compositions are designated composition 6-5.

In another embodiment of the sixth aspect, the present disclosure provides compositions according to composition 6-4, where the D-amino acid or derivative thereof is a D-serine analog; such compositions are designated composition 6-6.

In another embodiment of the sixth aspect, the present disclosure provides compositions according to composition 6-6, where the D-serine analog is an ester of D-serine, alkylated D-serine or a precursor of D-serine; such compositions are designated composition 6-7.

In another embodiment of the sixth aspect, the present disclosure provides compositions according to any one of compositions 6-1-6-7 where at least one of the one or more agents is an anti-psychotic; such compositions are designated composition 6-8.

In another embodiment of the sixth aspect, the present disclosure provides compositions according to composition 6-8, where the anti-psychotic is a phenothiazine; such compositions are designated composition 6-9.

In another embodiment of the sixth aspect, the present disclosure provides compositions according to composition 6-9, where the phenothiazine is chlorpromazine; such compositions are designated composition 6-10.

In another embodiment of the sixth aspect, the present disclosure provides compositions according to composition 6-8, where the anti-psychotic is a butyrophenone; such compositions are designated composition 6-11.

In another embodiment of the sixth aspect, the present disclosure provides compositions according to composition 6-11, where the butyrophenone is haloperidol; such compositions are designated composition 6-12.

In another embodiment of the sixth aspect, the present disclosure provides compositions according to composition 6-8, where the anti-psychotic is an atypical anti-psychotic; such compositions are designated composition 6-13.

In another embodiment of the sixth aspect, the present disclosure provides compositions according to composition 6-13, where the atypical anti-psychotic is chosen from clozapine, olanzapine, ziprasidone, risperidone, and quetiapine; such compositions are designated composition 6-14.

In another embodiment of the sixth aspect, the present disclosure provides compositions according to any one of compositions 6-1-6-14, where at least one of the one or more agents is an anti-cholinergic; such compositions are designated composition 6-15.

In another embodiment of the sixth aspect, the present disclosure provides compositions according to composition 6-15, where the anticholinergic is tacrine or donepezil; such compositions are designated composition 6-16.

In another embodiment of the sixth aspect, the present disclosure provides compositions according to any one of compositions 6-1-6-16, where the compound or salt of the present disclosure and the one or more agents are contained within the same unit dosage form; such compositions are designated composition 6-17.

In another embodiment of the sixth aspect, the present disclosure provides compositions according to any one of compositions 6-1-6-16, where the compound or salt of the present disclosure is contained in a first unit dosage form and the one or more agents are contained within a second unit dosage form; such compositions are designated composition 6-18.

In another embodiment of the sixth aspect, the present disclosure provides compositions according to any one of compositions 6-1-6-18, where the composition is contained within a package with instructions for using the composition; such compositions are designated composition 6-19.

Compounds of the present disclosure can exist as prodrugs. Prodrugs of compounds of any of the aspects of the present disclosure can also be prepared using synthetic methodologies known to those skilled in the art.

The kits of the seventh aspect of the present disclosure provide agents for preventing and/or treating neurological and/or psychiatric disorders and typically comprise one or more containers, where each container comprises a therapeutically effective amount of a compound of the present disclosure and, optionally, a therapeutically effective amount of an agent useful in the prevention and/or treatment of a neurological or psychiatric disorder; or a therapeutically effective amount of a composition of the present disclosure.

In some embodiments, the kits further comprise instructions for use of the kit, and in certain embodiments thereof, instructions for using the components of the kit to treat or prevent neurological and/or psychiatric disorders.

The kits of the present disclosure are packaged pharmaceutical products. The packaged pharmaceutical product comprises a compound of the present disclosure, for example, as a composition of the compound and a pharmaceutically acceptable carrier, excipient or diluent, and optionally one or more additional agents useful in the prevention and/or treatment of a neurological or psychiatric disorder, also, in certain embodiments, as a composition of the agent and a carrier, excipient or diluent. Certain packaged pharmaceutical products include instructions explaining how to use the product to treat one or more neurological or psychiatric disorders.

In an embodiment of the eighth aspect, the present disclosure provides methods where the neurological or psychiatric disorder is schizophrenia; such methods are designated method 8-1.

In another embodiment of the eighth aspect, the present disclosure provides methods where the neurological or psychiatric disorder is Alzheimer's disease; such methods are designated method 8-2.

In another embodiment of the eighth aspect, the present disclosure provides methods where the neurological or psychiatric disorder is dementia; such methods are designated method 8-3.

In another embodiment of the eighth aspect, the present disclosure provides methods of method 8-3, where the dementia is senile dementia; such methods are designated method 8-4.

In another embodiment of the eighth aspect, the present disclosure provides methods of method 8-3, where the dementia is dementia associated with Alzheimer's disease; such methods are designated method 8-5.

In another embodiment of the eighth aspect, the present disclosure provides methods where the neurological or psychiatric disorder is a bipolar disorder; such methods are designated method 8-6.

In another embodiment of the eighth aspect, the present disclosure provides methods where the neurological or psychiatric disorder is a mood disorder; such methods are designated method 8-7.

In another embodiment of the eighth aspect, the present disclosure provides methods where the neurological or psychiatric disorder is depression; such methods are designated method 8-8.

In another embodiment of the eighth aspect, the present disclosure provides methods of any one of methods 8-1-8-8, where the compound, salt or composition is administered orally; such methods are designated method 8-9.

In another embodiment of the eighth aspect, the present disclosure provides methods of any one of methods 8-1-8-9, where the compound, salt or composition is provided as a sustained release formulation; such methods are designated method 8-10.

In another embodiment of the eighth aspect, the present disclosure provides methods of any one of methods 8-1-8-10, further comprising administering one or more agents useful in the prevention and/or treatment of a neurological or psychiatric disorder; such methods are designated method 8-11.

In another embodiment of the eighth aspect, the present disclosure provides methods of method 8-11, where the administering is performed simultaneously; such methods are designated method 8-12.

In another embodiment of the eighth aspect, the present disclosure provides methods of method 8-11, where the administering is performed sequentially; such methods are designated method 8-13.

In another embodiment of the eighth aspect, the present disclosure provides methods of any one of methods 8-1-8-13, where the patient has been medically diagnosed with a neurological or psychiatric disorder; such methods are designated method 8-14.

In an embodiment of the ninth aspect, the present disclosure provides compositions, further comprising one or more agents useful in the prevention and/or treatment of a neurological or psychiatric disorder; such compositions are designated composition 9-1.

In another embodiment of the ninth aspect, the present disclosure provides compositions, further comprising one or more agents useful in the prevention and/or treatment of a neurological or psychiatric disorder, where the compound or salt thereof and the one or more agents are contained within the same unit dosage form; such compositions are designated composition 9-2.

In another embodiment of the ninth aspect, the present disclosure provides compositions, further comprising one or more agents useful in the prevention and/or treatment of a neurological or psychiatric disorder, where the compound or salt thereof is contained in a first unit dosage form and the one or more agents are contained within a second unit dosage form; such compositions are designated composition 9-3.

### DEFINITIONS

The substituents as denoted herein are written to be read “left to right,” unless preceded by a dash, which denotes the point of attachment of the substituent to the parent moiety (e.g., —S(O)₂NH₂ is bonded via the sulfur atom). For example, a substituent “R₁₆—(C₁-C₆)alkyl”, means an “R₁₆” group attached to a parent moiety via an alkyl group, as defined herein; therefore the bond between the parent moiety and the R₁₆—(C₁-C₆)alkyl group is to a carbon in the alkyl group. In another example, the substituent, R₁₆—(C₁-C₆)alkylthio, means an “R₁₆—(C₁-C₆)alkyl” group, as noted previously attached to a parent moiety via an sulfur atom; therefore the bond between the parent moiety and the R₁₆—(C₁-C₆)alkylthio group is to a sulfur atom, which itself is bonded to a carbon in the alkyl group.

The term “optionally substituted” as used herein, means the referenced moiety has a substituent group at any substitutable atom, i.e., the substitution only replaces a hydrogen atom with another substituent group and does not result in violating valence bonding at the substitutable atom (e.g., no carbon atoms that form 5 bonds). Further, the number of optionally substituted groups present on any optionally substituted moiety is limited by the number of substitutable atoms present in the moiety. For example a phenyl moiety has exactly 5 substitutable positions (i.e., one position for bonding the phenyl moiety to a parent structure) and therefore can only have up to 5 optionally substituted groups.

The term “alkenyl” as used herein, means a straight or branched chain hydrocarbon containing from 2 to 10 carbons, unless otherwise, defined, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy” as used herein, means an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkyl” as used herein, means a straight or branched chain hydrocarbon containing from 1 to 10 carbon atoms, unless otherwise defined. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylamino” as used herein, means an alkyl group, as defined herein, appended to the parent molecular moiety through an —N(H)— group Representative examples of alkylamino groups include, but are not limited to, methylamino, propylamino, and tert-butylamino.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl” as used herein, means an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkylthioalkyl” as used herein, means an alkylthio group, as defined herein, appended to the parent molecular moiety through an alkyl group, as defined herein. Representative examples of alkylthioalkyl include, but are not limited, methylthiomethyl and 2-(ethylthio)ethyl.

The term “alkynyl” as used herein, means a straight or branched chain hydrocarbon group containing from 2 to 10 carbon atoms, unless otherwise defined, and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “amidino” as used herein means a —C(NH)NH₂ group.

The term “amino” as used herein, means a —NH₂ group.

The term “aminoalkyl” as used herein, means at least one amino group, as defined herein, is appended to the parent molecular moiety through an alkyl group, as defined herein. Representative examples of hydroxyalkyl include, but are not limited to, aminomethyl, 2-aminoethyl, 3-aminopropyl, 2,3-diaminopentyl, and 2-ethyl-4-aminoheptyl.

The term “aryl,” as used herein, means a phenyl group or a bicyclic aryl ring or a tricyclic aryl ring. The aryl groups can be attached to the parent molecular moiety through any carbon atom within the aryl group while maintaining the proper valence. The bicyclic aryl ring consists of a phenyl group fused to a cycloalkyl group or a phenyl group fused to a cycloalkenyl group or a phenyl group fused to another phenyl group. Representative examples of the bicyclic aryl ring include, but are not limited to, 2,3-dihydro-1H-indenyl, 1H-indenyl, naphthyl, 7,8-dihydronaphthalenyl, and 5,6,7,8-tetrahydronaphthalenyl. The tricyclic aryl ring consists of the bicyclic aryl ring fused to a cycloalkyl group or the bicyclic aryl ring fused to a cycloalkyl group or the bicyclic aryl ring fused to another phenyl group. Representative examples of tricyclic aryl ring include, but are not limited to, anthracenyl, azulenyl, 9,10-dihydroanthracenyl, fluorenyl, and 4b,8a,9,10-tetrahydrophenanthrenyl.

The term “arylalkoxy” as used herein, means an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “aryloxy” as used herein, means an aryl group, as defined herein, appended to the parent molecular moiety through an oxygen atom. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “alkylaryl”, as used herein means an aryl residue having one or more alkyl groups attached thereto. Examples are tolyl and mesityl.

The terms “carbamoyl” and “carboxamido” as used herein mean a —C(O)NH₂ group.

The term “carbonyl” as used herein, means a —C(O)— group.

The term “carboxy” as used herein, means a —CO₂H group.

The term “carboxyalkoxy” as used herein, means a carboxy group, as defined herein, appended to the parent molecular moiety through an alkoxy group, as defined herein. Representative examples of carboxyalkoxy include, but are not limited to, carboxymethoxy, 2-carboxyethoxy, and 3-carboxypropyloxy.

The term “carboxyalkyl” as used herein, means a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkyl” as used herein, means a monocyclic, bicyclic, or tricyclic ring system having only carbon atoms in the rings. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1^{3,7}]decane (adamantane).

The term “dialkylamino” as used herein, means two alkyl groups, each independently selected and as defined herein, appended to the parent molecular moiety through a nitrogen atom. Representative examples of alkylamino groups include, but are not limited to, dimethylamino, (methyl)ethylamino, (hexyl)propylamino, and (methyl)tert-butylamino.

The term “formyl” as used herein, means a —C(O)H group.

The term “halo” or “halogen” as used herein, means chloro, bromo, iodo or fluoro. Preferred halogens within R₃₀ are fluoro, chloro, and bromo.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “haloalkylthio” as used herein, means a haloalkyl group, as defined herein, appended to the parent molecular moiety through a sulfur atom.

The term “heterocycloalkyl” as used herein, means a monocyclic heterocycle or a bicyclic heterocycle or a tricyclic heterocycle. The monocyclic heterocycle is a 3, 4, 5, 6 or 7 membered ring containing at least one heteroatom independently selected from the group consisting of O, N, and S. The 3 or 4 membered ring contains 1 heteroatom selected from the group consisting of O, N and S. The 5 membered ring contains zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a cycloalkyl, or a monocyclic heterocycle fused to a cycloalkenyl, or a monocyclic heterocycle fused to a monocyclic heterocycle, or a monocyclic heterocycle fused to a monocyclic heteroaryl. The bicyclic heterocycle is connected to the parent molecular moiety through any carbon atom or any nitrogen atom contained within the bicyclic heterocycle. Representative examples of bicyclic heterocycle include, but are not limited to, 1,3-benzodioxolyl, 1,3-benzodithiolyl, 2,3-dihydro-1,4-benzodioxinyl, 2,3-dihydro-1-benzofuranyl, 2,3-dihydro-1-benzothienyl, 2,3-dihydro-1H-indolyl, and 1,2,3,4-tetrahydroquinolinyl. The tricyclic heterocycle is a bicyclic heterocycle fused to a phenyl, or a bicyclic heterocycle fused to a cycloalkyl, or a bicyclic heterocycle fused to a cycloalkenyl, or a bicyclic heterocycle fused to a monocyclic heterocycle, or a bicyclic heterocycle fused to a monocyclic heteroaryl. The tricyclic heterocycle is connected to the parent molecular moiety through any carbon atom or any nitrogen atom contained within the tricyclic heterocycle. Representative examples of tricyclic heterocycle include, but are not limited to, 2,3,4,4a,9,9a-hexahydro-1H-carbazolyl, 5a,6,7,8,9,9a-hexahydrodibenzo[b,d]furanyl, and 5a,6,7,8,9,9a-hexahydrodibenzo[b,d]thienyl.

The term “heteroaryl,” as used herein, means a monocyclic heteroaryl or a bicyclic heteroaryl. The monocyclic heteroaryl is a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thiophenyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The bicyclic heteroaryl is connected to the parent molecular moiety through any carbon atom or any nitrogen atom contained within the bicyclic heteroaryl. Representative examples of bicyclic heteroaryl include, but are not limited to, benzimidazolyl, benzofuranyl, benzothiophenyl, benzoxadiazolyl, cinnolinyl, dihydroquinolinyl, dihydroisoquinolinyl, furopyridinyl, indazolyl, indolyl, isoquinolinyl, naphthyridinyl, quinolinyl, tetrahydroquinolinyl, and thienopyridinyl.

The term “heteroarylalkyl” as used herein, means a heteroaryl, as defined herein, appended to the parent molecular moiety through an alkyl group, as defined herein. Representative examples of heteroarylalkyl include, but are not limited to, fur-3-ylmethyl, 1H-imidazol-2-ylmethyl, 1H-imidazol-4-ylmethyl, 1-(pyridin-4-yl)ethyl, pyridin-3-ylmethyl, 6-chloropyridin-3-ylmethyl, pyridin-4-ylmethyl, (6-(trifluoromethyl)pyridin-3-yl)methyl, (6-(cyano)pyridin-3-yl)methyl, (2-(cyano)pyridin-4-yl)methyl, (5-(cyano)pyridin-2-yl)methyl, (2-(chloro)pyridin-4-yl)methyl, pyrimidin-5-ylmethyl, 2-(pyrimidin-2-yl)propyl, thien-2-ylmethyl, pyridinylmethyl, pyrimidinylethyl, and thien-3-ylmethyl.

The term “hydroxy” or “hydroxyl” as used herein, means an —OH group.

The term “hydroxyalkyl” as used herein, means at least one hydroxyl group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “hydroxyamino” as used herein, means an —N(H)OH group.

The term “mercapto” as used herein, means a —SH group.

The term “nitro” as used herein, means a —NO₂ group.

The term “oxo” as used herein means a ═O group.

The terms “thiophenyl” and “thienyl” as used herein mean a group of the formula

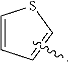

The term “acyl” as used herein means a group of from 1 to 20 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, and benzyloxycarbonyl. Lower-acyl refers to groups containing one to four carbons.

The term “acyloxy” as used herein means an acyl group, as defined herein attached to a parent moiety through an oxygen atom.

The term “acylamino” as used herein means an acyl group, as defined herein attached to a parent moiety through a —N(H)— group.

Certain compounds described in this application contain a tetrazolyl group. The tetrazolyl moiety exists in tautomeric equilibrium between the 1H-tetrazol-5-yl and the 2H-tetrazol-5-yl tautomers. It is to be understood that both the 1H-tetrazolyl and 2H-tetrazolyl tautomers and isomers are encompassed by such names, illustrations and descriptions as well.

Further, certain compounds described herein can exist as a mixture of tautomers, i.e., in tautomeric equilibrium. While one tautomer will typically predominate and be isolable, all tautomers are encompassed by the names, structures, and other descriptions herein as well. For example, sodium 6H-selenopheno[3,2-b]pyrrole-5-carboxylate can exist in at least two tautomeric forms which may be represented by the following structures:

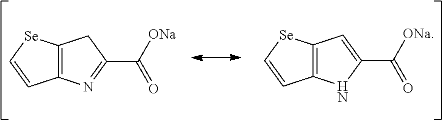

Each of these tautomeric forms of the compound is intended to be encompassed by the names and structures herein.

The term “prodrug”, as used herein refers to a derivative of an active compound (drug) that requires a transformation under the conditions of use, such as within the body, to release the active drug. Prodrugs are frequently, but not necessarily, pharmacologically inactive until converted into the active drug. Prodrugs are typically obtained by masking a functional group in the drug believed to be in part required for activity with a progroup (defined below) to form a promoiety which undergoes a transformation, such as cleavage, under the specified conditions of use to release the functional group, and hence the active drug. The cleavage of the promoiety can proceed spontaneously, such as by way of a hydrolysis reaction, or it can be catalyzed or induced by another agent, such as by an enzyme, by light, by acid, or by a change of or exposure to a physical or environmental parameter, such as a change of temperature. The agent can be endogenous to the conditions of use, such as an enzyme present in the cells to which the prodrug is administered or the acidic conditions of the stomach or it can be supplied exogenously.

A wide variety of progroups, as well as the resultant promoieties, suitable for masking functional groups in the active drugs to yield prodrugs are well-known in the art. For example, a hydroxyl functional group can be masked as a sulfonate, ester or carbonate promoiety, which can be hydrolyzed in vivo to provide the hydroxyl group. An amino functional group can be masked as an amide, carbamate, imine, urea, phosphenyl, phosphoryl or sulfenyl promoiety, which can be hydrolyzed in vivo to provide the amino group. A carboxyl group can be masked as an ester (including silyl esters and thioesters), amide or hydrazide promoiety, which can be hydrolyzed in vivo to provide the carboxyl group. Other specific examples of suitable progroups and their respective promoieties will be apparent to those of skill in the art.

### DAO Related Therapeutic Methods

The compounds (e.g., compounds which inhibit DAO) and compositions (e.g., pharmaceutical compositions) of the present disclosure are useful in methods for the prevention, treatment, control, amelioration, or reduction of risk of the diseases, disorders and conditions noted herein.

### Neurological and Psychiatric Disorders

The compounds of the present disclosure have utility in treating a variety of neurological and psychiatric disorders associated with glutamatergic neurotransmission dysfunction, including one or more of the following conditions or diseases: schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anesthetics, amphetamine and other psychostimulants and cocaine) psychosispsychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both the positive and the negative symptoms of schizophrenia and other psychoses; cognitive disorders including dementia (associated with AIDS, Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline, short term memory, loss of long term memory, mild cognitive impairment, cognitive impairment associated with hydrocephalus, cognitive and memory impairment associated with head injury or trauma (sometimes referred to amnesic disorder due to a general medical condition); anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD), attention deficit disorder (ADD), and conduct disorder; NMDA receptor-related disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de Ia Tourette's syndrome, epilepsy, muscular spasms and disorders associated with muscular spasticity or weakness including tremors; dyskinesias [including tremor (such as rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics), and dystonia (including generalized dystonia such as idiopathic dystonia, drug-induced dystonia, symptomatic dystonia and paroxysmal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; sleep disorders including insomnia and narcolepsy; neurodegenerative diseases and disorders, such as MLS (cerebellar ataxia), ataxia, amyotrophic lateral sclerosis, Down syndrome, status epilecticus, contusive injuries (e.g., spinal cord injury and head injury), viral infection induced neurodegeneration, (e.g., AIDS, encephalopathies); and neurotoxic injury that follows cerebral stroke, thromboembolic stroke, hemorrhagic stroke, cerebral ischemia, cerebral vasospasm, hypoglycemia amnesia, hypoxia, anoxia, perinatal asphyxia and cardiac arrest. The present disclosure provides a method for preventing and/or treating a neurological or psychiatric disorder comprising: administering to a patient in need thereof an effective amount of a compound of the present disclosure alone or in combination with another agent useful in the prevention and/or treatment of a neurological or psychiatric disorder. For example, in certain embodiments of the present disclosure, the neurological and psychiatric disorder is chosen from schizophrenia, bipolar disorder, depression including unipolar depression, seasonal depression and post-partum depression, premenstrual syndrome (PMS) and premenstrual dysphoric disorder (PDD), learning disorders, pervasive developmental disorder including autistic disorder, attention disorders including Attention-Deficit/Hyperactivity Disorder, autism, tic disorders including Tourette's disorder, anxiety disorders including phobia and post traumatic stress disorder, cognitive disorders associated with dementia, AIDS dementia, Alzheimer's, Parkinson's, Huntington's disease, spasticity, myoclonus, muscle spasm, tinnitus and hearing impairment and loss.

In one embodiment, the present disclosure provides a method for preventing and/or treating a cognitive disorder, comprising: administering to a patient in need thereof an effective amount of a compound of the present disclosure alone or in combination with another agent useful in the treatment of a cognitive disorder. Thus, the cognitive disorder may include, for example, dementia, delirium, amnestic disorders and age-related cognitive decline. The text revision of the fourth edition of the Diagnostic and Statistical Manual of Mental Disorders (DSM-IV-TR) (2000, American Psychiatric Association, Washington D.C.) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In another embodiment, the present disclosure provides a method for preventing and/or treating Alzheimer's Disease (AD) including the cognitive impairment associated with AD comprising: administering to a patient in need thereof an effective amount of a compound of the present disclosure alone or in combination with another agent useful in the prevention and/or treatment of AD. Methods for diagnosing AD are known in the art. For example, the National Institute of Neurological and Communicative Disorders and Stroke-Alzheimer's Disease- and the Alzheimer's Disease and Related Disorders Association (NINCDS-ADRDA) criteria can be used to diagnose AD (McKhann et al. 1984 Neurology 34:939-944). The patient's cognitive function can be assessed by the Alzheimer's Disease Assessment Scale-cognitive subscale (ADAS-cog; Rosen et al., 1984, Am. J. Psychiatry 141:1356-1364).

In another embodiment, the present disclosure provides a method for preventing and/or treating an anxiety disorder, comprising: administering to a patient in need thereof an effective amount of a compound of the present disclosure alone or in combination with another agent useful in the prevention and/or treatment of one or more anxiety disorders. Anxiety disorders include but are not limited to generalized anxiety disorder, obsessive-compulsive disorder and panic attack. The text revision of the fourth edition of the Diagnostic and Statistical Manual of Mental Disorders (DSM-IV-TR) (2000, American Psychiatric Association, Washington D.C.) provides a diagnostic tool that includes anxiety disorders are generalized anxiety disorder, obsessive-compulsive disorder and panic attack. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

In another embodiment, the present disclosure provides a method for preventing and/or treating schizophrenia or psychosis comprising: administering to a patient in need thereof an effective amount of a compound of the present disclosure alone or in combination with another agent useful in the prevention and/or treatment of schizophrenia or psychosis. Schizophrenia or psychosis pathologies include paranoid, disorganized, catatonic or undifferentiated schizophrenia and substance-induced psychotic disorder. The text revision of the fourth edition of the Diagnostic and Statistical Manual of Mental Disorders (DSM-IV-TR) (2000, American Psychiatric Association, Washington D.C.) provides a diagnostic tool that includes paranoid, disorganized, catatonic or undifferentiated schizophrenia and substance-induced psychotic disorder. As used herein, the term “schizophrenia or psychosis” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

In another embodiment, the present disclosure provides a method for preventing and/or treating substance-related disorders and addictive behaviors, comprising: administering to a patient in need thereof an effective amount of a compound of the present disclosure alone or in combination with another agent useful in the prevention and/or treatment of one or more substance-related disorders or addictive behaviors. Substance-related disorders and addictive behaviors include but are not limited to persisting dementia, persisting amnestic disorder, psychotic disorder or anxiety disorder induced by substance abuse; and tolerance of, dependence on or withdrawal from substances of abuse. The text revision of the fourth edition of the Diagnostic and Statistical Manual of Mental Disorders (DSM-IV-TR) (2000, American Psychiatric Association, Washington D.C.) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

In another embodiment, the present disclosure provides a method for treating obesity or eating disorders associated with excessive food intake and complications associated therewith, comprising: administering to a patient in need thereof an effective amount of a compound of the present disclosure alone or in combination with one or more other agents useful in the prevention and/or treatment of obesity or eating disorders associated with excessive food intake and complications associated therewith. Obesity is included in the tenth edition of the International Classification of Diseases and Related Health Problems (ICD-IO) (1992 World Health Organization) as a general medical condition. The text revision of the fourth edition of the Diagnostic and Statistical Manual of Mental Disorders (DSM-IV-TR) (2000, American Psychiatric Association, Washington D.C.) provides a diagnostic tool that includes obesity in the presence of psychological factors affecting medical condition. As used herein, the term “obesity or eating disorders associated with excessive food intake” includes treatment of those medical conditions and disorders described in ICD-10 and DSM-IV-TR. The skilled artisan will recognize that there are alternative nomenclatures, nosologies and classification systems for general medical conditions, and that these systems evolve with medical and scientific progress. Thus the term “obesity or eating disorders associated with excessive food intake” is intended to include like conditions and disorders that are described in other diagnostic sources.

### Pain and Inflammation

The compounds of the present disclosure are useful in the prevention and/or treatment of diseases and conditions in which pain and/or inflammation predominates, including chronic and acute pain conditions. Such conditions include, for example, rheumatoid arthritis; osteoarthritis; post-surgical pain; musculoskeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynecological pain, for example, dysmenorrhoea, pain associated with cystitis and labor pain; pain associated with nerve and root damage, such as pain associated with peripheral nerve disorders, for example, nerve entrapment and brachial plexus avulsions, amputation/phantom limb pain, peripheral neuropathies, tic douloureux, atypical facial pain, nerve root damage, and arachnoiditis; itching conditions including pruritis, itch due to hemodialysis, and contact dermatitis; pain (as well as broncho-constriction and inflammation) due to exposure (e.g., via ingestion, inhalation, or eye contact) of mucous membranes to capsaicin and related irritants such as tear gas, hot peppers or pepper spray; neuropathic pain conditions such as diabetic neuropathy, chemotherapy-induced neuropathy, neuralgia (for example, including post-herpetic neuralgia and trigeminal neuralgia), sciatica, back pain, non-specific lower back pain, multiple sclerosis pain, fibromyalgia, HIV-related neuropathy, pain related to chronic alcoholism, hypothyroidism, uremia, or vitamin deficiencies, pain related to compression of the nerves (i.e. Carpal Tunnel Syndrome), and pain resulting from physical trauma, amputation/phantom limb pain), cancer, toxins or chronic inflammatory conditions; “non-painful” neuropathies; complex regional pain syndromes; pain associated with carcinoma, often referred to as cancer pain; central nervous system pain, such as pain due to spinal cord or brain stem damage, lower back pain, sciatica and ankylosing spondylitis; gout; scar pain; irritable bowel syndrome; inflammatory bowel disease; urinary incontinence including bladder detrusor hyper-reflexia and bladder hypersensitivity; respiratory diseases including chronic obstructive pulmonary disease (COPD), chronic bronchitis, cystic fibrosis and asthma; autoimmune diseases; and immunodeficiency disorders.

The present disclosure provides a method for preventing and/or treating a disorder associated with pain and/or inflammation comprising: administering to a patient in need thereof an effective amount of a compound of the present disclosure alone or in combination with another agent useful in the prevention and/or treatment of a disorder associated with pain and/or inflammation. For example, in certain embodiments of the present disclosure, the disorder associated with pain and/or inflammation is chosen from bone and joint pain (osteoarthritis), repetitive motion pain, dental pain, cancer pain, myofascial pain (muscular injury, fibromyalgia), perioperative pain (general surgery, gynecological), and chronic pain

In one embodiment, the present disclosure provides a method for preventing and/or treating neuropathic pain comprising: administering to a patient in need thereof an effective amount of a compound of the present disclosure alone or in combination with another agent useful in the prevention and/or treatment of neuropathic pain. Neuropathic pain syndromes include but are not limited to diabetic neuropathy, chemotherapy-induced neuropathy, neuralgia (for example, including post-herpetic neuralgia (pain occurring after Shingles) and trigeminal neuralgia), sciatica, back pain, non-specific lower back pain, multiple sclerosis pain, fibromyalgia, HIV-related neuropathy, pain related to chronic alcoholism, hypothyroidism, uremia, or vitamin deficiencies, pain related to compression of the nerves (i.e. Carpal Tunnel Syndrome), and pain resulting from or associated with physical trauma, (e.g., amputation/phantom limb pain), stroke, spinal chord injury, cancer, toxins or chronic inflammatory conditions. The symptoms of neuropathic pain are incredibly heterogeneous. Patients with neuropathic pain typically describe sensations such as burning, spontaneous shooting, lancinating, or electric pain. Other pain sensations commonly experienced include: “pins and needles”/tingling (paraesthesias and dysesthesias), pain from a stimulus that is usually not painful (allodynia), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia), an absence of or deficit in selective sensory pathways (hypoalgesia) and sympathetic pain (a syndrome of sustained burning pain, allodynia and hyperpathia (e.g., following a traumatic nerve lesion).

### Combination Therapy

The compounds of the present disclosure may be used in combination with one or more other agents in the treatment, prevention, control, amelioration, or reduction of risk of diseases or conditions for which compounds of the present disclosure or the other agents may have utility, where the combination of the agents together are safer or more effective than either agent alone. Such other agent(s) may be administered, by a route and in an amount commonly used therefore, contemporaneously or sequentially with a compound of the present disclosure. When a compound of the present disclosure is used contemporaneously with one or more other agents, a pharmaceutical composition in unit dosage form containing such other agents and the compound of the present disclosure may be utilized. Combination therapy may also include therapies in which the compound of the present disclosure and one or more other agents are administered on different overlapping schedules. It is also contemplated that when used in combination with one or more other active ingredients, the compounds of the present disclosure and the other active ingredients may be used in lower doses than when each is used singly. Accordingly, the pharmaceutical compositions of the present disclosure may include those that contain one or more other active ingredients, in addition to a compound of the present disclosure. The above combinations include combinations of a compound of the present disclosure not only with one other active compound, but also with two or more other active compounds.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X—Y—X, X—X—Y, Y—X—Y, Y—Y—X, X—X—Y—Y, etc.

The weight ratio of the compound of the present disclosure to additional active ingredients may be varied and will depend upon the effective dose of each ingredient. Generally, an effective dose of each will be used. Thus, for example, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used.

In such combinations the compound of the present disclosure and other active agents may be administered separately or in conjunction. In addition, the administration of one element may be prior to, concurrent to, or subsequent to the administration of other agent(s).

Accordingly, the compounds of the present disclosure may be used alone or in combination with other agents which are known to be beneficial in the subject indications or other agents that affect receptors or enzymes that either increase the efficacy, safety, convenience, or reduce unwanted side effects or toxicity of the compounds of the present disclosure. The compound of the present disclosure and the other agent may be co-administered, either in concomitant therapy or in a fixed combination.

### Specific Combination Therapy Agents

The compounds of the present disclosure may be employed in combination with one or more D-amino acids or suitable derivatives thereof useful in the prevention and/or treatment of a neurological or psychiatric disorders such as D-phenylalanine, para-fluoro-D-phenyl alanine, D-(N-trifluoroacetyl-4-fluorophenylalanine), D-leucine, D-alanine, D-cycloserine and D-serine or D/L mixtures thereof, a D-serine analog (e.g., a salt of D-serine, an ester of D-serine, alkylated D-serine, or a precursor of D-serine).

The compounds of the present disclosure may be employed in combination with one or more agents useful in the prevention and/or treatment of a neurological or psychiatric disorder chosen from: 5-HT1_{A} agonists or antagonists (e.g., 5-HT1_{A} partial agonists), 5HT-2 antagonists, 5HT6 antagonist (e.g., SB271046 (GSK), SB737552 (S-8510, GSK), SR 57667 (Sanofi Aventis), SR 57746 (Sanofi Aventis), A2a adenosine receptor antagonists, alpha2/serotonin-2/seratonin-3 antagonists, alpha-adrenoreceptor antagonists, ampakines (e.g., CX516 (Ampalex™, Cortex Pharmaceuticals)), anti-amyloid antibodies, anticholinergics, antidepressants, anti-psychotic agent, antioxidants, anxiolytic, atypical anti-depressants, barbiturates, benzodiazepines, benzodiazepines, beta-secretase inhibitors, cholinergic agonists, COMT inhibitors such as entacapone, conjugated estrogen (e.g., Premarin, Wyeth), corticotropin releasing factor (CRF) antagonists, corticotropin releasing factor (CRF) antagonists, cyclopyrrolones, dopamine receptor agonists and pharmaceutically acceptable salts thereof such as alentemol, bromocriptine, fenoldopam, lisuride, naxagolide, pergolide, pramipexole, alentemol hydrobromide, bromocriptine mesylate, fenoldopam mesylate, naxagolide hydrochloride and pergolide mesylate, dopamine reuptake inhibitors, dual serotonin and norepinephrine reuptake inhibitors, gamma-secretase inhibitors, HMG-CoA reductase inhibitors (statins such as atorvastatin, rosuvastatin, simvastatin, and fluvastatin), hypnotics, imidazopyridines, inhibitors of glycine transporter GIyTl activity (e.g., ALX 5407, Allelix Neuroscience), M1 muscarinic receptor antagonists, melatonergic agents, melatonin agonists and antagonists, minor tranquilizers, MOA-B inhibitors, monoamine oxidase inhibitors (MAOIs), neurokinin-1 receptor antagonists, NMDA receptor antagonists, norepinephrine reuptake inhibitors (including tertiary amine tricyclics and secondary amine tricyclics), pyrazolopyrimidines, reversible inhibitors of monoamine oxidase (RIMAs), sedatives, selective norepinephrine, selective serotonin reuptake inhibitors (SSRIs), serotonin and noradrenaline reuptake inhibitors (SNRIs), serotonin receptor antagonists, serotonin-2 antagonism/reuptake inhibitors, and TNF-alpha antagonists (e.g., CPI-1189, CAS Registry No. 183619-38-7). Specific agents include: adinazolam, allobarbital, alonimid, alprazolam, amitriptyline, amobarbital, amoxapine, aprepitant, bentazepam, benzoctamine, betaine, biperiden (optionally as its hydrochloride or lactate salt), brotizolam, bupropion, busprione, butabarbital, butalbital, capuride, carbocloral, chloral, chloral hydrate, chlordiazepoxide, clomipramine, clonazepam, cloperidone, clorazepate, clorethate, cyprazepam, desipramine, dexclamol, diazepam, dichloralphenazone, diphenhydramine, divalproex, doxepin, duloxetine, estazolam, ethchlorvynol, etomidate, fenobam, flesinoxan, flunitrazepam, fluoxetine, flurazepam, fluvoxamine, fosazepam, galantamine (sold as Razadyne Razadyne ER Reminyl Nivalin Janssen Pharmaceutica), gepirone, glutethimide, halazepam, hydroxyzine, imipramine, ipsapirone, isocarboxazid, leteprinim (Neotrofin® NeoTherapeutics), levodopa (with or without a selective extracerebral decarboxylase inhibitor such as carbidopa or benserazide), lithium, lorazepam, lormetazepam, maprotiline, mecloqualone, melatonin, mephobarbital, meprobamate, methaqualone, midaflur, midazolam, moclobemide, nefazodone, nisobamate, nitrazepam, nortriptyline, oxazepam, paraldehyde, paroxetine, pentobarbital, perlapine, phenelzine, phenobarbital, phenserine (a phenylcarbamate of physostigmine Axonyx), prazepam, promethazine, propofol, protriptyline, quazepam, quetiapine, reclazepam, rivastigmine (sold as Exelon Novartis), roletamide, secobarbital, selegiline, sertraline, suproclone, temazepam, tetrabenazine, tracazolate, tranylcypromaine, trazodone, trepipam, triazolam, tricetamide, triclofos, trihexyphenidyl (benzhexol) hydrochloride, trimetozine, trimipramine, uldazepam, venlafaxine, viloxazine, vitamin E/tocopherol, zaleplon, zolazepam, and zolpidem.

The compounds of the present disclosure may be employed in combination with an anticholinergic such as tacrine or donepezil hydrochloride (Aricept®, Eisai Co., Japan).

The compounds of the present disclosure may be employed in combination with an anti-psychotic agent (e.g., a neuroleptic agent). Typical antipsychotics include phenothiazines such as acetophenazine, chlorpromazine (Thorazine), fluphenazine (Prolixin), levomepromazine (Nozinan), mesoridazine, perphenazine (Trilafon), prochlorperazine (Compazine), promazine, thioridazine (Mellaril), trifluoperazine (Stelazine), and triflupromazine (Vesprin); thioxanthenes such as chlorprothixene, flupenthixol (Depixol and Fluanxol), thiothixene (Navane), and zuclopenthixol (Clopixol and Acuphase); butyrophenones such as azaperone, benperidol, droperidol, haloperidol (Haldol), and pimozide (Orap), and other agents such as loxapine, molindone, and sulforidazine. Atypical antipsychotics include as amisulpride, aripiprazole (Abilify®), bifeprunox, clozapine (Clozaril®), melperone, olanzapine (Zyprexa® also, (Symbyax™) when combined with Fluoxetine (Prozac®), paliperidone (Invega®), quetiapine (Seroquel®), risperidone (Risperdal®), sertindole (Serlect®), sulpiride, ziprasidone (Geodon®), and zotepine. The anti-psychotic agent when used in combination with the compound of the present disclosure may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are Commonly used in a non-salt form.

The compound of the present disclosure may be administered in conjunction with the use of physical methods such as with light therapy or electrical stimulation.

The compounds of the present disclosure can be administered in combination with a DAO or DDO inhibitor or antagonists such as those described in U.S. Application 20030166554 (see for example, paragraphs 128-157), hereby incorporated by reference. Suitable DDO inhibitors can include: aminoethylcysteine-ketimine (AECK, thialysine ketimine, 2H-1,4-thiazine-5,6-dihydro-3-carboxylic acid, S-aminoethyl-L-cysteine ketimine, 2H-1,4-Thiazine-3-carboxylic acid, 5,6-dihydro-); aminoethylcysteine (thialysine); cysteamine; pantetheine; cystathionine; and S-adenosylmethionine.

The compounds of the present disclosure may be employed in combination with a compound useful in the treatment of pain, for example an NSAID such as ibuprofen, an antinociceptive agent such as an NR2B antagonist, a COX2 inhibitor such as ARCOXIA or a sodium channel blocker.

### Administration

The compounds, salts, and compositions of the present disclosure may be administered by oral, parenteral (e.g., intramuscular, intraperitoneal, intravenous, intracerebroventricular (ICV), intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the disclosure are effective for use in humans.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present disclosure encompass any composition made by admixing a compound of the present disclosure and a pharmaceutically acceptable carrier.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

Tablets contain the active ingredient in admixture with non-toxic pharmaceutically acceptable excipients that are suitable for the manufacture of tablets. The tablets may be uncoated or they may be coated by known techniques to delay disintegration and absorption in the gastrointestinal tract and thereby provide a sustained action over a longer period or may be tablets that disperse when added to water. Compositions for oral use may also be presented as hard gelatin capsules where the active ingredients are mixed with an inert solid diluent, for example, calcium carbonate, calcium phosphate or kaolin, or as soft gelatin capsules where the active ingredient is mixed with water or an oil medium, for example peanut oil, liquid paraffin, or olive oil. Aqueous suspensions, oily suspensions, dispersible powders or granules, oil-in-water emulsions, and sterile injectable aqueous or oleaginous suspension may be prepared by standard methods known in the art.

The agents, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

The agent can be in the form of a pharmaceutically acceptable salt. Such salts are prepared from pharmaceutically acceptable non-toxic bases including inorganic bases and organic bases. Examples of salts derived from inorganic bases include aluminum, ammonium, calcium, copper, ferric, ferrous, lithium, magnesium, manganic salts, manganous, potassium, sodium, zinc, and the like. In some embodiments, the salt can be an ammonium, calcium, magnesium, potassium, or sodium salt. Examples of salts derived from inorganic bases include aluminum, ammonium, calcium, copper, ferric, ferrous, lithium, magnesium, manganic salts, manganous, potassium, sodium, zinc, and the like. In some embodiments, the salt can be an ammonium, calcium, magnesium, potassium, or sodium salt. Examples of salts derived from pharmaceutically acceptable organic non-toxic bases include salts of primary, secondary, and tertiary amines, benethamine, N,N′-dibenzylethylenediamine, diethylamine, 2-diethylaminoethanol, 2-dimethylaminoethanol, diethanolamine, ethanolamine, ethylenediamine, N-ethylmorpholine, N-ethylpiperidine, epolamine, glucamine, glucosamine, histidine, hydrabamine, isopropylamine, lysine, methylglucamine, meglumine, morpholine, piperazine, piperidine, polyamine resins, procaine, purines, theobromine, triethylamine, trimethylamine, tripropylamine, and trolamine, tromethamine. Examples of other salts include tris, arecoline, arginine, barium, betaine, bismuth, chloroprocaine, choline, clemizole, deanol, imidazole, and morpholineethanol. In one embodiment are tris salts. In another embodiment are calcium, d-serine (monosodium), potassium, tetramethylammonium, tris, ammonium, benethamine, benzathine, choline, clemizole, deanol, dicyclohexylamine, diethanolamine, diethylamine, diethylaminoethanol, epolamine, ethanolamine, ethylenediamine, ethylpropylammonium, hydrabamine, imidazole, 1-lysine, magnesium, meglumine, morpholineethanol, piperazine, pyridine, sodium, trolamine, and zinc salts.

The agents can be administered orally, e.g., as a tablet or cachet containing a predetermined amount of the active ingredient, pellet, gel, paste, syrup, bolus, electuary, slurry, capsule; powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered by captisol delivery technology, rectal suppository or parenterally.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein. The pharmaceutical compositions may include a “pharmaceutically acceptable inert carrier”, and this expression is intended to include one or more inert excipients, which include starches, polyols, granulating agents, microcrystalline cellulose, diluents, lubricants, binders, disintegrating agents, and the like. If desired, tablet dosages of the disclosed compositions may be coated by standard aqueous or nonaqueous techniques, “Pharmaceutically acceptable carrier” also encompasses controlled release means.

Compositions of the present disclosure may also optionally include other therapeutic ingredients, anti-caking agents, preservatives, sweetening agents, colorants, flavors, desiccants, plasticizers, dyes, and the like. Any such optional ingredient must be compatible with the compound to insure the stability of the formulation.

The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as:

BINDERS: alginic acid, cellulose and its derivatives (e.g. ethyl cellulose, cellulose acetate, carboxymethyl cellulose, carboxymethyl cellulose calcium, sodium carboxymethyl cellulose), citric acid monohydrate, corn starch, gelatin, guar gum, hydroxymethyl cellulose, hydroxypropyl cellulose, hydroxypropyl methyl cellulose, methyl cellulose, microcrystalline cellulose (e.g., AVICEL™ such as AVICEL-PH-101™, -103™, and 105™ sold by FMC Corporation, Marcus Hook, Pa. USA), natural and synthetic gums such as acacia, other alginates, other starches, polyethylene oxide, polyvinyl alcohol, polyvinyl pyrrolidone, potato starch, powdered tragacanth, pre-gelatinized starch (e.g., STARCH 1500® and STARCH 1500 LM®, sold by Colorcon), sodium alginate, or mixtures thereof;
FILLERS: aluminum magnesium hydroxide, aluminum oxide, calcium carbonate (e.g., granules or powder), calcium dihydroxide, calcium sulfate (e.g., granules or powder), dextrates, dextrose, dibasic calcium phosphate, dibasic calcium phosphate anhydrous, fructose (granules or powder), honey, hydrous lactose, iron oxides (e.g., yellow, black, red, e.g., ferric oxide), kaolin, lactose, lactose and aspartame, lactose and cellulose, lactose and microcrystalline cellulose, lactose anhydrate, lactose monohydrate, magnesium aluminate, magnesium carbonate, magnesium hydroxide, maltodextrin, maltose, mannitol, microcrystalline cellulose, microcrystalline cellulose & guar gum, molasses, powdered cellulose, pre-gelatinized starch, silicic acid, silicic anhydride, silicified microcrystalline cellulose, sodium chloride, sorbitol, soybean lecithin, starch, sucrose, talc, triacetin, tribasic calcium phosphate, xanthan gum, or mixtures thereof;
DISINTEGRANTS: agar-agar, alginic acid, calcium carbonate, clays, croscarmellose sodium, crospovidone, gums (like gellan), lactose monohydrate, low-substituted hydroxypropyl cellulose, microcrystalline cellulose, other algins, other celluloses, other starches, polacrilin potassium, potato or tapioca starch, povidone, pre-gelatinized starch, simethicone emulsion, sodium starch glycolate, or mixtures thereof;
SURFACTANTS: Tween 80 or polyoxyethylene-polyoxypropylene copolymer, polyoxyethylene sorbitan, or mixtures thereof;
LUBRICANTS: a coagulated aerosol of synthetic silica (Degussa Co. Plano Tex. USA), a pyrogenic silicon dioxide (CAB-O-SIL, Cabot Co., Boston, Mass. USA), agar, calcium stearate, ethyl laurate, ethyl oleate, glycerin, hydrogenated vegetable oil (e.g., peanut oil, cottonseed oil, sunflower oil, sesame oil, olive oil, corn oil and soybean oil), light mineral oil, magnesium stearate, mannitol, mineral oil, other glycols, palmitic acid, polyethylene glycol, sodium lauryl sulfate, sodium stearyl fumarate, sorbitol, stearic acid, syloid silica gel (AEROSIL 200, W.R. Grace Co., Baltimore, Md. USA), talc, vegetable based fatty acids lubricant, zinc stearate, or mixtures thereof;
ANTI-CAKING AGENTS: calcium silicate, magnesium silicate, silicon dioxide, colloidal silicon dioxide, talc, or mixtures thereof;
ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, polysorbate, sorbic acid, thimersol, thymo, or mixtures thereof;
COATING AGENTS: candelilla wax, carnuba wax, cellulose acetate phthalate, ethylcellulose, gelatin, gellan gum, hydroxypropyl cellulose, hydroxypropyl methyl cellulose phthalate, hydroxypropyl methylcellulose (hypromellose), maltodextrin, methacrylates, methylcellulose, microcrystalline cellulose and carrageenan, microcrystalline wax, pharmaceutical glaze, polyethylene glycol (e.g., polyethylene glycol 8000, polyethylene glycol 3000), polyvinyl acetate phthalate, shellac, sodium carboxymethyl cellulose, sucrose, titanium dioxide, or mixtures thereof; COLORANTS: FD&C blue no. 1, D&C yellow #10 aluminum lake, FD&C yellow #6/sunset yellow FCF aluminum lake, FD&C carmine aluminum lake and FD&C blue #1, or mixtures thereof; and
ANTIOXIDANTS: butylated hydroxyanisole, sodium ascorbate, sodium metabisulfate, malic acid, citric acid, ascorbic acid, butylated hydroxytoluene, vitamin C, propyl gallate, or mixtures thereof.

The formulation can also include other excipients and categories thereof including but not limited to L-histidine, Pluronic®, Poloxamers (such as Lutrol® and Poloxamer 188), ascorbic acid, glutathione, permeability enhancers (e.g., lipids, sodium cholate, acylcarnitine, salicylates, mixed bile salts, fatty acid micelles, chelators, fatty acid, surfactants, medium chain glycerides), protease inhibitors (e.g., soybean trypsin inhibitor, organic acids), pH lowering agents and absorption enhancers effective to promote bioavailability (including but not limited to those described in U.S. Pat. No. 6,086,918 and U.S. Pat. No. 5,912,014), creams and lotions (like maltodextrin and carrageenans); materials for chewable tablets (like dextrose, fructose, lactose monohydrate, lactose and aspartame, lactose and cellulose, maltodextrin, maltose, mannitol, microcrystalline cellulose and guar gum, sorbitol crystalline); parenterals (like mannitol and povidone); plasticizers (like dibutyl sebacate, plasticizers for coatings, polyvinylacetate phthalate); powder lubricants (like glyceryl behenate); soft gelatin capsules (like sorbitol special solution); spheres for coating (like sugar spheres); spheronization agents (like glyceryl behenate and microcrystalline cellulose); suspending/gelling agents (like carrageenan, gellan gum, mannitol, microcrystalline cellulose, povidone, sodium starch glycolate, xanthan gum); sweeteners (like aspartame, aspartame and lactose, dextrose, fructose, honey, maltodextrin, maltose, mannitol, molasses, sorbitol crystalline, sorbitol special solution, sucrose); wet granulation agents (like calcium carbonate, lactose anhydrous, lactose monohydrate, maltodextrin, mannitol, microcrystalline cellulose, povidone, starch), caramel, carboxymethylcellulose sodium, cherry cream flavor and cherry flavor, citric acid anhydrous, citric acid, confectioner's sugar, D&C Red No. 33, D&C Yellow #10 Aluminum Lake, disodium edetate, ethyl alcohol 15%, FD& C Yellow No. 6 aluminum lake, FD&C Blue #1 Aluminum Lake, FD&C Blue No. 1, FD&C blue no. 2 aluminum lake, FD&C Green No. 3, FD&C Red No. 40, FD&C Yellow No. 6 Aluminum Lake, FD&C Yellow No. 6, FD&C Yellow No. 10, glycerol palmitostearate, glyceryl monostearate, indigo carmine, lecithin, manitol, methyl and propyl parabens, mono ammonium glycyrrhizinate, natural and artificial orange flavor, pharmaceutical glaze, poloxamer 188, Polydextrose, polysorbate 20, polysorbate 80, polyvidone, pregelatinized corn starch, pregelatinized starch, red iron oxide, saccharin sodium, sodium carboxymethyl ether, sodium chloride, sodium citrate, sodium phosphate, strawberry flavor, synthetic black iron oxide, synthetic red iron oxide, titanium dioxide, and white wax.

Solid oral dosage forms may optionally be treated with coating systems (e.g., Opadry® fx film coating system, for example Opadry® blue (OY-LS-20921), Opadry® white (YS-2-7063), Opadry® white (YS-1-7040), and black ink (S-1-8106).

In the treatment of conditions which require inhibition of D-amino acid oxidase activity an appropriate dosage level will vary from 0.005 mg to 10 g/day orally and may generally be about 0.01 to 500 mg per kg patient body weight per day which can be administered in single or multiple doses. The dosage level can be about 0.1 to about 250 mg/kg per day, about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. Tablets or other forms of presentation provided in discrete units may conveniently contain an amount of compound described herein which is effective at such dosage or as a multiple of the same, for instance, units containing 5 mg to 500 mg, usually around 10 mg to 200 mg. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day. This dosage regimen may be adjusted to provide the optimal therapeutic response. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

A dosage unit (e.g., an oral dosage unit) can include from, for example, 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg) of a compound described herein. In certain embodiments the dosage unit and daily dose are equivalent. In various embodiments, the dosage unit is administered with food at anytime of the day, without food at anytime of the day, with food after an overnight fast (e.g., with breakfast), at bedtime after a low fat snack. In various embodiments, the dosage unit is administered once a day, twice a day, three times a day, four times a day.

Combining two or more active ingredients in single dosage form results in the possibility of chemical interactions between the active drug substances. For example, acidic and basic active ingredients can react with each other and acidic active ingredients can facilitate the degradation of acid labile substances. Thus, in certain dosage forms, acidic and basic substances can be physically separated as two distinct or isolated layers in a compressed tablet, or in the core and shell of a press-coated tablet. Additional agents that are compatible with acidic as well as basic substances, have the flexibility of being placed in either layer. In certain multiple layer compositions at least one active ingredient can be enteric-coated. In certain embodiments thereof at least one active ingredient can be presented in a controlled release form. In certain embodiments where a combination of three or more active substances are used, they can be presented as physically isolated segments of a compressed multilayer tablet, which can be optionally film coated.

The therapeutic combinations described herein can be formulated as a tablet or capsule comprising a plurality of beads, granules, or pellets. All active ingredients including the vitamins of the combination are formulated into granules or beads or pellets that are further coated with a protective coat, an enteric coat, or a film coat to avoid the possible chemical interactions. Granulation and coating of granules or beads is done using techniques well known to a person skilled in the art. At least one active ingredient can present in a controlled release form. Finally these coated granules or beads are filled into hard gelatin capsules or compressed to form tablets.

The therapeutic combinations described herein can be formulated as a capsule comprising microtablets or minitablets of all active ingredients. Microtablets of the individual agents can be prepared using well known pharmaceutical procedures of tablet making like direct compression, dry granulation or wet granulation. Individual microtablets can be filled into hard gelatin capsules. A final dosage form may comprise one or more microtablets of each individual component. The microtablets may be film coated or enteric coated.

The therapeutic combinations described herein can be formulated as a capsule comprising one or more microtablets and powder, or one or more microtablets and granules or beads. In order to avoid interactions between drugs, some active ingredients of a said combination can be formulated as microtablets and the others filled into capsules as a powder, granules, or beads. The microtablets may be film coated or enteric coated. At least one active ingredient can be presented in controlled release form.

The therapeutic combinations described herein can be formulated where the active ingredients are distributed in the inner and outer phase of tablets. In an attempt to divide chemically incompatible components of proposed combination, few interacting components are converted in granules or beads using well known pharmaceutical procedures in prior art. The prepared granules or beads (inner phase) are then mixed with outer phase comprising the remaining active ingredients and at least one pharmaceutically acceptable excipient. The mixture thus comprising inner and outer phase is compressed into tablets or molded into tablets. The granules or beads can be controlled release or immediate release beads or granules, and can further be coated using an enteric polymer in an aqueous or non-aqueous system, using methods and materials that are known in the art.

The therapeutic combinations described herein can be formulated as single dosage unit comprising suitable buffering agent. All powdered ingredients of said combination are mixed and a suitable quantity of one or more buffering agents is added to the blend to minimize possible interactions.

The agents described herein, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means.

Suitable pharmaceutical compositions in accordance with the present disclosure will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences, 18th Ed., Mack Publishing Company, 1995.

### Kits

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g., preventing or treating one or more of the diseases and disorders described herein). The kit may optionally comprise a second pharmaceutical composition comprising one or more additional agents described herein for cotherapy use, a pharmaceutically acceptable carrier, vehicle or diluent. The pharmaceutical composition comprising the compound described herein, and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### Synthesis of Compounds

The compounds of the invention may be prepared by use of known chemical reactions and procedures. Representative methods for synthesizing compounds of the invention are presented below. It is understood that the nature of the substituents required for the desired target compound often determines the preferred method of synthesis. One skilled in the art will recognize that certain proposed reaction conditions may necessitate the use of protecting groups to prevent undesired side-reactions. Suitable methods for protecting functional group is described, for example, in Greene and Wuts, Protective Groups in Organic Synthesis, 3rd ed., John Wiley & Sons: New York (1999), which is hereby incorporated by reference in its entirety. All variable groups of these methods are as described in the generic description if they are not specifically defined below.

Representative methods for preparation of the compounds of the present disclosure are outlined below in Schemes 1-7. The present compounds having a bicyclic core comprising a pyrrole ring fused to a selenophene or selenazole ring, can generally be prepared according to the methodology shown in Scheme 1.

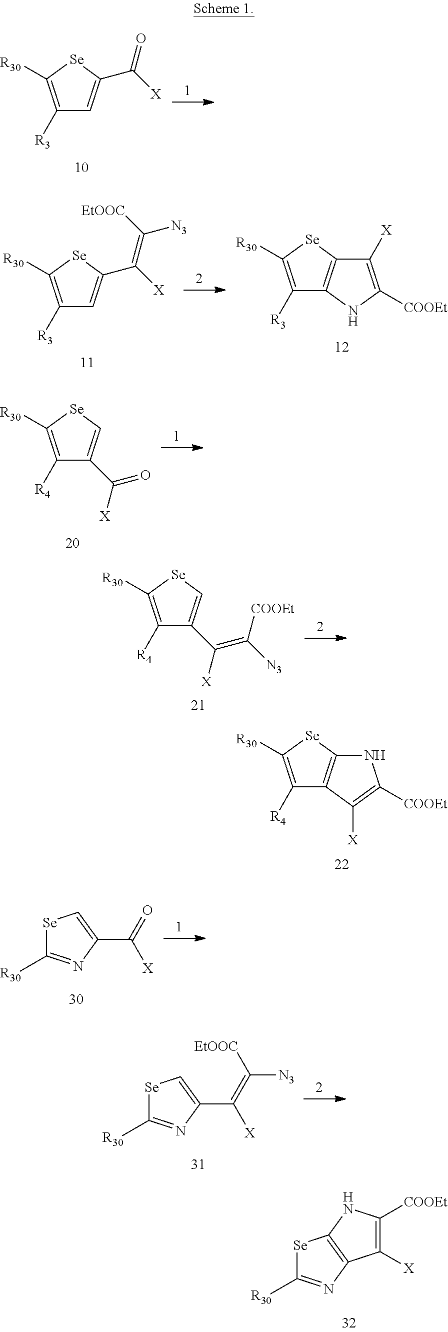

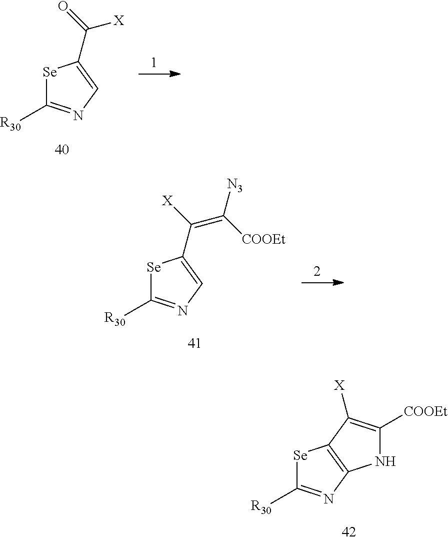

In each instance, a formyl-selenophene (10, 20) or acyl-selenazole (30, 40) can be reacted in a first step (1) with an alkyl azidoacetate (e.g., ethyl azidoacetate) in the presence of a base (e.g., sodium ethoxide), to form intermediate products 11, 21, 31, and 41, respectively. Each of the intermediates can be cyclyzed in a second step (2) by heating the same in a high boiling solvent such as xylenes or ethanol to give the desired bicyclic products 12, 22, 32, and 42. For example, the conditions described in Welch and Philips, Heterocyclic Commun. 1999, 5, 305-310; Shafiee et al., J. Heterocyclic Chem. 1979, 16, 1563-1566; and Koketsu and Ishihara, Curr. Org. Synth. 2007, 4, 15-29, can be used in preparing the compounds described herein.

Compounds 12, 22, 32, and 42 where X is halogen can be formed from the corresponding compound 12, 22, 32, or 42, respectively, where X is hydrogen, by halogenation of the pyrrole ring at the 3-position with a suitable halogenating agent such as chlorine, bromine, N-fluoropyridinium triflate, N-bromosuccinimide, N-chlorosuccinimide, N-iodosuccinimide, PCl₅, PPh₃Br₂, or PPh₃Cl₂.

Compounds 12, 22, 32, and 42 where X is cyano can be formed from the corresponding compound 12, 22, 32, or 42, respectively, where X is halogen, by treatment with a cyanide source, such as CuCN in DMF.

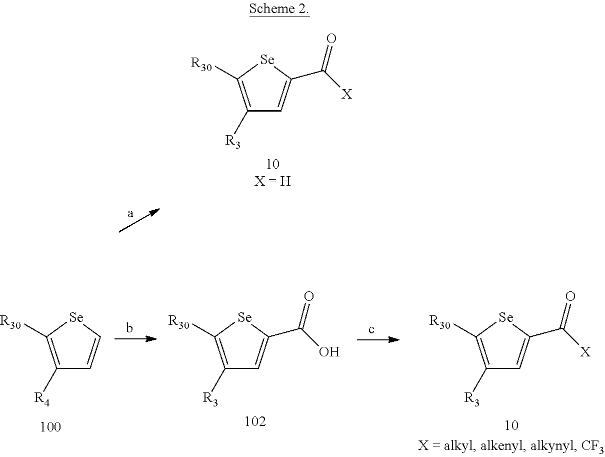

Compound 10 of Scheme 1 can be prepared as illustrated in Scheme 2. Starting with selenophene 100, the 2-formylselenophene compound, 10 (X═H), can be prepared directly by reaction, in step (a), with formylating reagents such as POCl₃ in N,N-dimethylformamide. Alternatively, selenophene 100 can be reacted with a strong base such as n-butyl lithium, s-butyl lithium t-butyl lithium, or phenyl lithium to form a 2-lithioselenophene intermediate which can be quenched by addition of N,N-dimethylformamide to yield compound 10 (X═H). In another example, selenophene 100 can be converted to aldehyde 10 (X═H) by halogenation (e.g., bromination) at the 2-position with a suitable halogenating agent, such as chlorine, bromine, N-bromosuccinimide, N-chlorosuccinimide, N-iodosuccinimide, PPh₃Br₂, PPh₃Cl₂, PPh₃ and iodine, to yield a 2-halo-intermediate. The 2-halo-intermediate can be reacted with carbon monoxide in the presence of a palladium catalyst (e.g., a palladium (0) catalyst such as, but not limited to tetrakis(triphenylphosphine) palladium(0), or a palladium (II) catalyst, such as, but not limited to, palladium acetate, dichloro bis(triphenylphosphine)palladium, and a hydrogen source, such as hydrogen gas or a silane (e.g., triethylsilane) to yield aldehyde 10 (X═H). See, for example, Ashfield and Barnard, Org. Process Res. Dev. 2007, 11, 39-43.

Compound 10 where X is alkyl, alkenyl, alkynyl, or trifluoromethyl, among others, can be prepared staring from the same starting selenophene, 100. Step (b) of Scheme 2 illustrates the conversion of selenophene 100 to its 2-carboxy-derivative. Such a conversion can be accomplished according to a number of methods, including, but not limited to, formation of the 2-lithio-derivative, as discussed previously for step (a), followed by quenching of the lithio-intermediate with carbon dioxide. If desired, the 2-lithio-intermediate can be converted to the corresponding Grignard reagent by the addition of, for example, anhydrous MgCl₂ or MgBr₂ prior to reaction with carbon dioxide. Alternatively, selenophene 100 can be halogenated to the 2-halo-derivative, as discussed above, followed by reaction of the 2-halo-intermediate with carbon monoxide in the presence of a palladium catalyst (as described previously) and an alcohol or water. In the case where water is utilized, the 2-carboxyselenophene intermediate, 102, is prepared directly; in the case where an alcohol is used (e.g., ethanol), the corresponding alkyl 2-selenophene carboxylate is formed and can be hydrolyzed according to methods known to those skilled in the art to intermediate 102. For example, the methods described in U.S. Pat. No. 3,988,358 can be utilized for preparation of compound 102 via reaction with carbon monoxide.

In step (c) of Scheme 2, intermediate 102 is converted to acyl-selenophene compound 20. Such a conversion can be achieved by reaction of intermediate 102 with an agent such as oxalyl chloride or thionyl chloride to give the acid chloride derivative of 102; the acid chloride derivative can be reacted with an organocuprate reagent of the form X₂CuLi to give product 20, where X is alkyl, alkenyl, alkynyl, or trifluoromethyl, among others. The organocuprate reagent of the form X₂CuLi can generally be formed by reaction of 2 equivalents of an organolithium compound (XLi) with a Cu(I) compound, such as CuI.

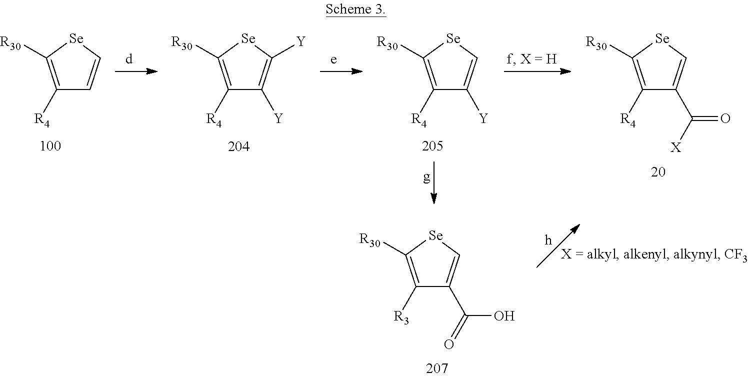

Starting with the same selenophene compound, 100, the regioisomeric formyl- or acyl-selenophene compound, 20, can be prepared as generally illustrated in Scheme 3. Compound 200 can be halogenated (e.g., brominated) in step (d) to yield dihalo-intermediate 204 (Y=halogen), using a halogenating agent as described above. Step (e) reacts intermediate 204 with one equivalent of a strong, non-nucleophilic base, such as n-butyl lithium, s-butyl lithium, or t-butyl lithium, to form the 2-lithio-derivative of 204 via a metal-halogen exchange. Quenching of the 2-lithio-derivative of 204 with a protic compound such as methanol, ethanol, or water, gives the 3-halo intermediate 205 (Y=halogen). Finally, formyl-selenophene compound 20 (X═H) can be prepared in step (f) by reaction of intermediate 205 with carbon monoxide in the presence of a palladium catalyst and hydrogen source as described above, or by a second metal-halogen exchange reaction to form the 3-lithio-derivative of 205, followed by quenching with N,N-dimethylformamide.

Compound 20, where X is alkyl, alkenyl, alkynyl, or trifluoromethyl, among others, can be formed by generation of compound 206 from 205 by quenching of the 3-lithio-derivative of 205, formed by metal-halogen exchange as described above, with carbon dioxide, also as described above. Alternatively, compound 205 can be reacted with carbon monoxide and a palladium catalyst in the presence of an alcohol or water, as described previously, to prepare compound 206. Finally, compound 20 can be formed from compound 206 in step (h) according to methods described for step (c) of Scheme 2.

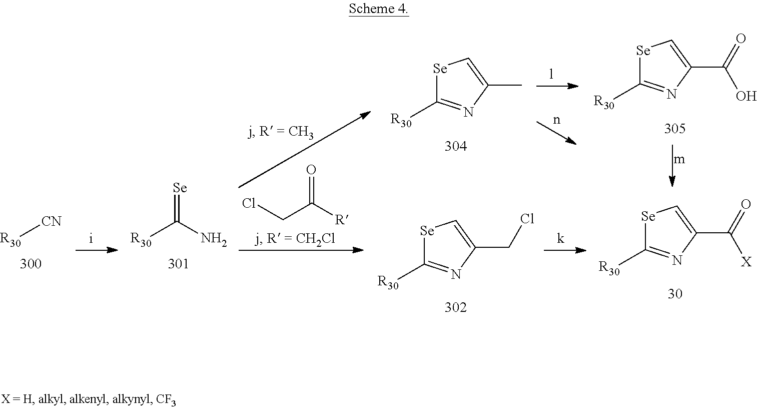

Intermediate selenazole compound 30 can be prepared starting from nitrile compound 300, as shown in Scheme 4. Compound 300 can be converted in step (i) of Scheme 4, to selenamide 301 by reaction with an appropriate selenating agent, such as, but not limited to, [PhP(Se)(μ-Se)]₂ (Woolins' reagent), P₂Se₅, H₂Se, Al₂Se₃, NaSeH, tris(trimethylsilyl)monoselenophosphate, or potassium selenobenzoate. For example, the methods described in Hua et al., Org. Lett. 2006, 8, 5251-5254; Bethke et al., Tetrahedron Lett. 2003, 44, 6911-6913; and Saravanan et al., Tetrahedron Lett. 2004, 45, 681-683, can be utilized for step (i) of Scheme 4.

Selenazole 302 can be prepared by reaction of intermediate 301 with a chloroacetate compound of the formula ClCH₂C(O)R′, where R′ is CH₂Cl (see, Shafiee et al., supra). Compound 30, where X═H, can be formed from 302 by reaction of 302 with sulfuric acid to form the 4-hydroxymethyl-derivative of compound 302, followed by oxidation of the 4-hydroxymethyl-derivative of 302 to compound 30 (X═H), by, for example, a Swern oxidation (dimethyl sulfoxide and oxalyl chloride, followed by a base, such as triethylamine) or with MnO₂.

Alternatively, compound 30 (X═H) can be formed by reaction of compound 301 with a chloroacetate compound of the formula ClCH₂C(O)R′, where R′ is CH₃ to give the 4-methyl compound 304 in step (j) of Scheme 4. Compound 30 where X is hydrogen can be formed directly from compound 304 by oxidation of the 4-methyl group of 304 to the aldehyde with CrO₃ in Ac₂O, a s shown in step (n).

Compound 30 where X is alkyl, alkenyl, alkynyl, or trifluoromethyl, among others, can be prepared from compound 304 by oxidation with potassium permanganate or sodium periodate to carboxylic acid 306, as shown in step (1) of Scheme 4. Finally, compound 305 can be converted to compound 30 in step (m) according to methods described previously for step (c) of Scheme 2.

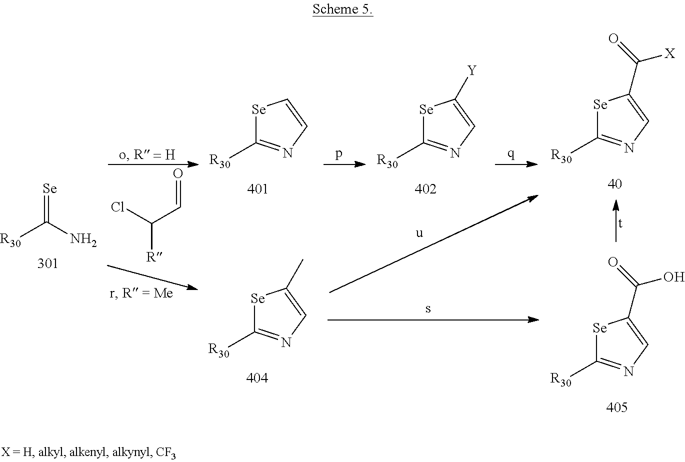

Intermediate selenazole compound 40 can be prepared according to the methods shown in Scheme 5. In step (o) of Scheme 5, selenamide compound 301 (prepared as described above) can be converted to selenazole 401 by reaction with chloroacetaldehyde in an analogous reaction as described for step (j) of Scheme 4. Selenazole 401 can be selectively halogenated at the 5-position with a suitable halogenating agent, as noted with respect to step (a) of Scheme 2 to give compound 402 (Y=halogen). Finally, compound 402 can be converted to compound 40 (X═H) in step (q), according to methods described for step (f) of Scheme 3.

Alternatively, compound 40 (X═H) can be prepared from compound 301 by reaction with 2-chloropropionaldehyde in an analogous reaction as described for step (j) of Scheme 4 to give the 5-methylselenazole compound 404. Then, in step (u), compound 40 (X═H) can be prepared from compound 404 by oxidation of the 5-methyl group as described in step (n) of Scheme 4.

Compound 40 where X is alkyl, alkenyl, alkynyl, or trifluoromethyl, among others, can be prepared from compound 404 by oxidation of the methyl group to the carboxylic acid derivative 405 in step (s) as described in step (1) of Scheme 4, followed by conversion of the carboxylic acid to compound 40 in step (t) as described in step (c) of Scheme 2.

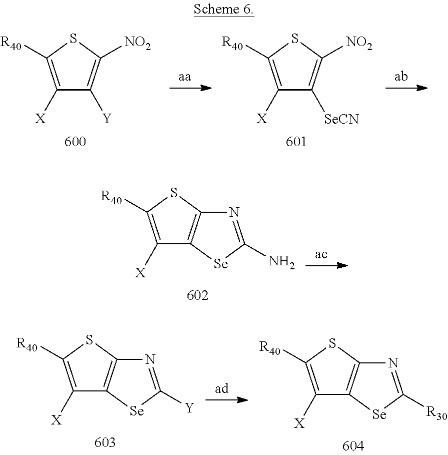

The various thienoselenazoles described herein, such as compound 604, can be prepared according the methods illustrated in Scheme 6. Starting with compound 600, where Y is halogen, nucleophilic aromatic substitution of the halide for a selenocyanate (step (aa)) by reaction with potassium selenocyanate in N,N-dimethylformamide can yield compound 601. Reduction of the nitro group in compound 601 to the amine in step (ab) can result in intramolecular cyclization through reaction of the newly introduced amino group with the selenocyanate to form the fused selenazole ring of intermediate compound 602. The amino group of intermediate 602 can be converted to a variety of groups according to methods familiar to one skilled in the art. For example, as shown in step (ac), the 2-amino group can be converted to a halo group through formation of a diazonium intermediate with isoamyl nitrite followed by treatment with a copper halide salt CuY₂, for example, CuBr₂ or CuCl₂, to give compound 603, where Y is halogen. Compound 603 can be converted via palladium-catalyzed coupling techniques known to those skilled in the art to yield product 604 in step (ad).

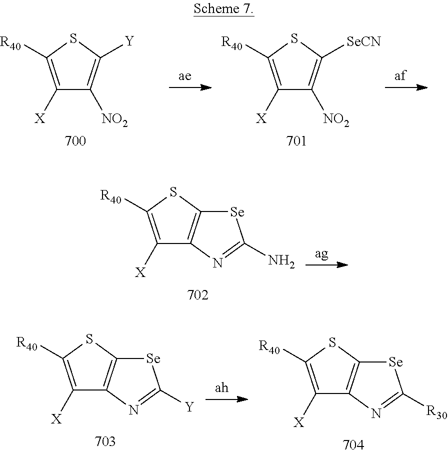

Analagously, regioisomeric thienoselenazoles, such as compound 704, can be prepared according the methods illustrated in Scheme 7. Starting with compound 700, where Y is halogen, nucleophilic aromatic substitution of the halide for a selenocyanate in step (ae) by reaction with potassium selenocyanate in N,N-dimethylformamide can yield compound 701. Reduction of the nitro group in compound 701 to the amine in step (af) can result in intramolecular cyclization through reaction of the newly introduced amino group with the selenocyanate to form the fused selenazole ring of intermediate compound 702. The amino group of intermediate 702 can be converted to a variety of functional groups according to methods familiar to one skilled in the art. For example, as shown in step (ag), the amino group can be converted to a halo group through formation of a diazonium intermediate with isoamyl nitrite followed by treatment with a copper halide salt CuY₂, for example, CuBr₂ or CuCl₂, to give compound 703, where Y is halogen. Compound 703 can be converted via palladium-catalyzed coupling techniques known to those skilled in the art to yield product 704 in step (ah).

Those having skill in the art will recognize that the starting materials and reaction conditions may be varied, the sequence of the reactions altered, and additional steps employed to produce compounds encompassed by the invention, as demonstrated by the following examples. In some cases, protection of certain reactive functionalities may be necessary to achieve some of the above transformations. In general, the need for such protecting groups as well as the conditions necessary to attach and remove such groups will be apparent to those skilled in the art of organic synthesis.

Compounds of the invention were named using Chemdraw version 10.0 (developed by CambridgeSoft available at cambridgesoft.com) or were given names which appeared to be consistent with Chemdraw version 10.0.

The invention is illustrated further by the following examples which are not to be construed as limiting the invention in scope or spirit to the specific procedures described in them.

### Example 1

### Preparation of 4H-selenopheno[3,2-b]pyrrole-5-carboxylate Salts

### Example 1a

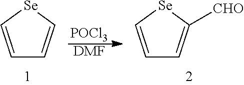

Selenophene 1 (5 g, 38 mmol) was dissolved in dichloromethane (4 mL) and N,N-dimethylformamide (2.78 g, 38 mmol, 3 mL) was added followed by POCl₃ (5.83 g, 38 mmol, 3.5 mL). The reaction was heated in a sealed tube to 65° C. for 1.5 h, after which time it is cooled to room temperature and poured over 100 mL of ice mixed with 1 g of potassium acetate. The mixture was then heated to reflux for 2 hours, cooled to room temperature, and the product was extracted with diethyl ether (3×100 mL) and vacuum distilled at 100° C. to afford 2.70 g of a clear oil selenophene-2-carbaldehyde that is 99% pure by ¹H NMR. ¹H NMR (400 MHz, CCl₃) δ 8.64 (m, 1H), 8.14 (dd, 1H, J=1.2, 4 Hz, 1H), 7.50 (dd, 1H, J=3.6, 4 Hz, 1H) ppm.

### Example 1b

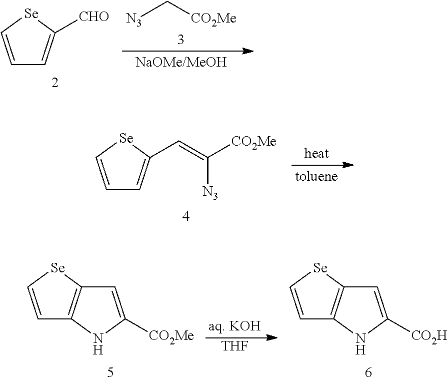

Selenophene-2-carbaldehyde (2) (2.70 g, 16.9 mmol) and methyl 2-azidoacetate (3) (7.76 g, 67.5 mmol) were dissolved in 100 mL of methanol and cooled to −10° C. To this solution was added 14.6 mL (67.5 mmol) of a 25% sodium methoxide solution in methanol, dropwise, via addition funnel (about one drop every 4 seconds). After the addition was complete, the reaction was allowed to warm slowly to room temperature and then stirred for 2.5 hours at room temperature. TLC (50% ethyl acetate in hexanes) displayed consumption of starting material, and the reaction was poured over iced saturated ammonium chloride, extracted with ethyl acetate (3×50 mL), dried over anhydrous sodium sulfate, filtered, and concentrated in vacuo to afford (Z)-methyl 2-azido-3-(selenophen-2-yl)acrylate (4) (3.99 g, 92% yield). (Z)-methyl 2-azido-3-(selenophen-2-yl)acrylate (4) (3.99 g, 15.6 mmol) was dissolved in toluene (15 mL) and heated to reflux (120° C.). When the reaction was judged complete by TLC (90 minutes), it was cooled to room temperature and 50% of the toluene was removed in vacuo. The resulting solid was filtered and dried resulting in (1.39 g, 6.09 mmol 40% yield) of methyl 4H-selenopheno[3,2-b]pyrrole-5-carboxylate (5). ¹H NMR (400 MHz, CD₃OD) δ 7.91 (d, J=5.6 Hz, 1H), 7.22 (d, J=5.2 Hz, 1H), 7.18 (s, 1H), 3.84 (s, 1H) ppm; MS [M+H]⁺ 229.

Methyl 4H-selenopheno[3,2-b]pyrrole-5-carboxylate (5) ((1.39 g, 6.09 mmol) was dissolved in THF (10 mL) and Water (10 mL). Potassium hydroxide (0.68 g, 12.18 mmol) was added and the reaction was divided into three and microwaved at 100° C. for 600 seconds. The completed reactions were pooled and quenched with 3N HCl (4 mL). The THF was slowly removed in vacuo and the resulting solid was collected via filtration yielding 4H-selenopheno[3,2-b]pyrrole-5-carboxylic acid (6) (1.10 g, 84% yield). ¹H NMR (400 MHz, CD₃OD) δ 7.86 (d, J=5.6 Hz, 1H), 7.22 (d, J=6.0 Hz, 1H), 7.0 (s, 1H) ppm; MS [M+H]⁺ 215.

### Example 1c

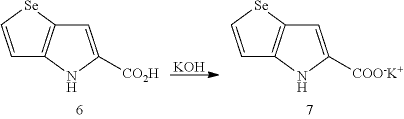

4H-selenopheno[3,2-b]pyrrole-5-carboxylic acid (6) (0.09 g, 0.44 mmol) was dissolved in THF (2 mL). To this solution was added 1N potassium hydroxide (0.44 mL, 0.4 mmol) at room temperature. The reaction was stirred for 5 minutes and the solvent was removed in vacuo. 0.1 g (0.4 mmol, 90%) of potassium 4H-selenopheno[3,2-b]pyrrole-5-carboxylate (7) was recovered. ¹H NMR (400 MHz, d-DMSO) δ 7.55 (d, J=5.6 Hz, 1H), 7.12 (d, J=5.6 Hz, 1H), 6.5 (s, 1H) ppm.

### Example 1d

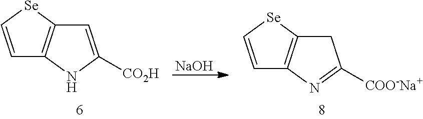

4H-selenopheno[3,2-b]pyrrole-5-carboxylic acid (6) (0.05 g, 0.23 mmol) was dissolved in THF (2 mL). To this solution was added 1N sodium hydroxide (0.23 mL, 0.23 mmol) at room temperature. The reaction was stirred for 5 minutes, cooled for 1 h at −30° C. and the solvent was removed in vacuo. 0.04 g (0.16 mmol, 68%) of sodium 6H-selenopheno[3,2-b]pyrrole-5-carboxylate (8) was recovered. ¹H NMR (400 MHz, CD₃CN) δ 7.72 (d, J=5.6 Hz, 1H), 7.63 (d, J=5.6 Hz, 1H), 4.20 (m, 2H) ppm.

### Example 2

The following compounds are prepared essentially according to the synthetic procedures employed in the above examples and outlined in the above schemes.

| Compound | | |
|---|---|---|
| # | Structure | Name |
| 1 | 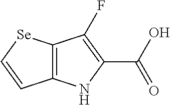 | 6-fluoro-4H- selenopheno[3,2- b]pyrrole-5-carboxylic acid; |
| 2 | 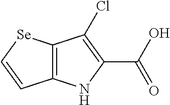 | 6-chloro-4H- selenopheno[3,2- b]pyrrole-5-carboxylic acid; |
| 3 | 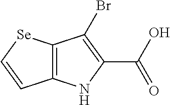 | 6-bromo-4H- selenopheno[3,2- b]pyrrole-5-carboxylic acid; |
| 4 | 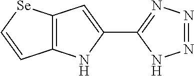 | 5-(1H-tetrazol-5-yl)- 4H-selenopheno[3,2- b]pyrrole; |
| 5 | 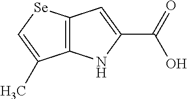 | 3-methyl-4H- selenopheno[3,2- b]pyrrole-5-carboxylic acid; |
| 6 | 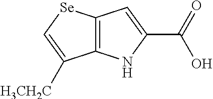 | 3-ethyl-4H- selenopheno[3,2- b]pyrrole-5-carboxylic acid; |
| 7 | 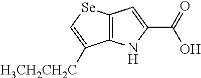 | 3-propyl-4H- selenopheno[3,2- b]pyrrole-5-carboxylic acid; |
| 8 | 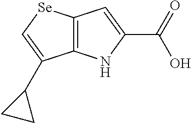 | 3-cyclopropyl-4H- selenopheno[3,2- b]pyrrole-5-carboxylic acid; |
| 9 | 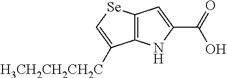 | 3-butyl-4H- selenopheno[3,2- b]pyrrole-5-carboxylic acid; |
| 10 | 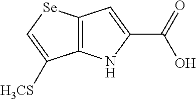 | 3-methylthio-4H- selenopheno[3,2- b]pyrrole-5-carboxylic acid; |
| 11 | 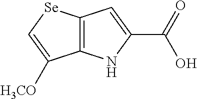 | 3-methoxy-4H- selenopheno[3,2- b]pyrrole-5-carboxylic acid; |
| 12 | 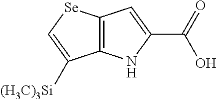 | 3-(trimethylsilyl)-4H- selenopheno[3,2- b]pyrrole-5-carboxylic acid; |
| 13 | 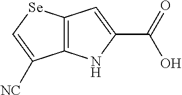 | 3-cyano-4H- selenopheno[3,2- b]pyrrole-5-carboxylic acid; |
| 14 | 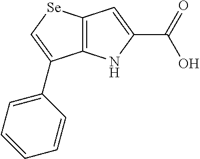 | 3-phenyl-4H- selenopheno[3,2- b]pyrrole-5-carboxylic acid; |
| 15 | 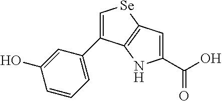 | 3-(3- hydroxyphenyl)-4H- selenopheno[3,2- b]pyrrole-5-carboxylic acid; |
| 16 | 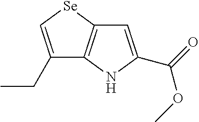 | methyl 3-ethyl-4H- selenopheno[3,2- b]pyrrole-5- carboxylate; |
| 17 | 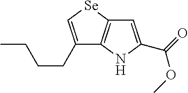 | methyl 3-butyl-4H- selenopheno[3,2- b]pyrrole-5- carboxylate; |
| 18 | 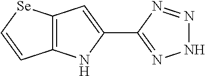 | 5-(2H-tetrazol-5-yl)- 4H-selenopheno[3,2- b]pyrrole; |
| 19 | 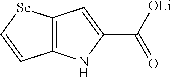 | lithium 4H- selenopheno[3,2- b]pyrrole-5- carboxylate; |
| 20 | 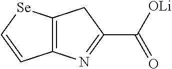 | lithium 6H- selenopheno[3,2- b]pyrrole-5- carboxylate; |
| 21 | 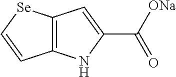 | sodium 4H- selenopheno[3,2- b]pyrrole-5- carboxylate; |
| 22 | 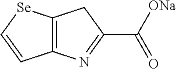 | sodium 6H- selenopheno[3,2- b]pyrrole-5- carboxylate; |
| 23 | 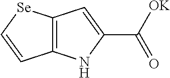 | potassium 4H- selenopheno[3,2- b]pyrrole-5- carboxylate |
| 24 | 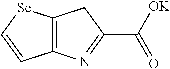 | potassium 6H- selenopheno[3,2- b]pyrrole-5- carboxylate |
| 25 | 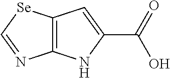 | 4H-pyrrolo[2,3- d][1,3]selenazole-5- carboxylic acid; |
| 26 | 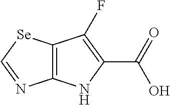 | 6-fluoro-4H-pyrrolo [2,3-d][1,3] selenazole-5- carboxylic acid; |
| 27 | 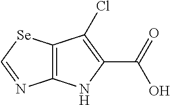 | 6-chloro-4H-pyrrolo [2,3-d][1,3] selenazole-5- carboxylic acid; |
| 28 | 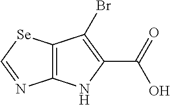 | 6-bromo-4H-pyrrolo [2,3-d][1,3] selenazole-5- carboxylic acid; |
| 29 | 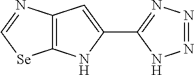 | 5-(1H-tetrazol-5-yl)- 4H-pyrrolo[3,2- d][1,3]selenazole; |
| 30 | 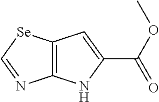 | methyl 4H-pyrrolo [2,3-d] selenazole-5- carboxylate; |
| 31 | 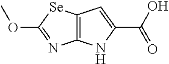 | 2-methoxy-4H- pyrrolo[2,3- d]selenazole-5- carboxylic acid; |
| 32 | 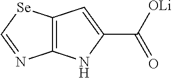 | lithium 4H-pyrrolo [2,3-d][1,3] selenazole-5- carboxylate; |
| 33 | 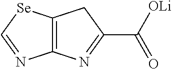 | lithium 6H-pyrrolo [2,3-d][1,3] selenazole-5- carboxylate; |
| 34 | 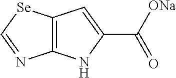 | sodium 4H-pyrrolo [2,3-d][1,3] selenazole-5- carboxylate; |
| 35 | 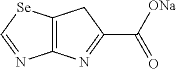 | sodium 6H-pyrrolo [2,3-d][1,3] selenazole-5- carboxylate; |
| 36 | 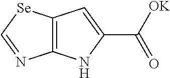 | potassium 4H- pyrrolo[2,3- d][1,3]selenazole-5- carboxylate; |
| 37 | 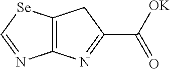 | potassium 6H- pyrrolo[2,3- d][1,3]selenazole-5- carboxylate; |
| 38 | 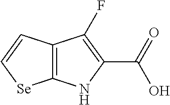 | 4-fluoro-6H- selenopheno[2,3- b]pyrrole-5-carboxylic acid; |
| 39 | 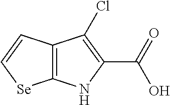 | 4-chloro-6H- selenopheno[2,3- b]pyrrole-5-carboxylic acid; |
| 40 | 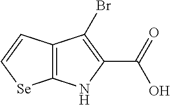 | 4-bromo-6H- selenopheno[2,3- b]pyrrole-5-carboxylic acid; |
| 41 | 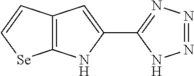 | 5-(1H-tetrazol-5-yl)- 6H-selenopheno[2,3- b]pyrrole; |
| 42 | 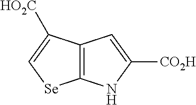 | 6H-seleno[2,3-b] pyrrole-3,5- dicarboxylic acid; |
| 43 | 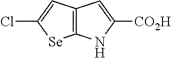 | 2-chloro-6H-seleno [2,3-b]pyrrole-5- carboxylic acid; |
| 44 | 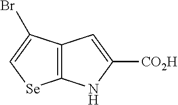 | 3-bromo-6H-seleno [2,3-b]pyrrole-5- carboxylic acid; |
| 45 | 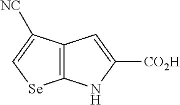 | 3-cyano-6H-seleno [2,3-b]pyrrole-5- carboxylic acid; |
| 46 | 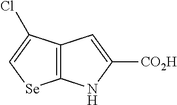 | 3-chloro-6H-seleno [2,3-b]pyrrole-5- carboxylic acid; |
| 47 | 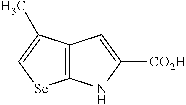 | 3-methyl-6H-seleno [2,3-b]pyrrole-5- carboxylic acid; |
| 48 | 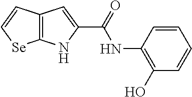 | N-(2-hydroxyphenyl)- 6H-seleno[2,3-b] pyrrole-5- carboxamide; |
| 49 | 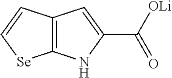 | lithium 4H- selenopheno[2,3- b]pyrrole-5- carboxylate; |
| 50 | 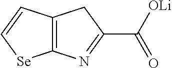 | lithium 6H- selenopheno[2,3- b]pyrrole-5- carboxylate; |
| 51 | 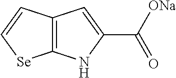 | sodium 4H- selenopheno[2,3- b]pyrrole-5- carboxylate; |
| 52 | 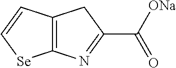 | sodium 6H- selenopheno[2,3- b]pyrrole-5- carboxylate |
| 53 | 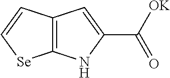 | potassium 4H- selenopheno[2,3- b]pyrrole-5- carboxylate; |
| 54 | 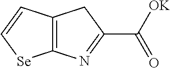 | potassium 6H- selenopheno[2,3- b]pyrrole-5- carboxylate |
| 55 | 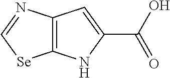 | 4H-pyrrolo[3,2- d][1,3]selenazole-5- carboxylic acid; |
| 56 | 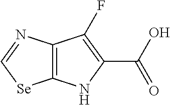 | 6-fluoro-4H-pyrrolo [3,2-d][1,3] selenazole-5- carboxylic acid; |
| 57 | 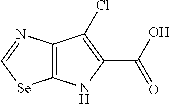 | 6-chloro-4H-pyrrolo [3,2-d][1,3] selenazole-5- carboxylic acid; |
| 58 | 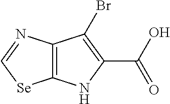 | 6-bromo-4H-pyrrolo [3,2-d][1,3] selenazole-5- carboxylic acid; |
| 59 | 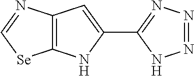 | 5-(1H-tetrazol-5-yl)- 4H-pyrrolo[3,2- d][1,3]selenazole; |
| 60 | 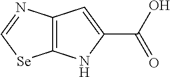 | 4H-pyrrolo[3,2- d][1,3]selenazole-5- carboxylic acid; |
| 61 | 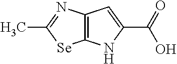 | 2-methyl-4H-pyrrolo [3,2-d][1,3] selenazole-5- carboxylic acid; |
| 62 | 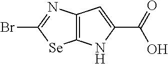 | 2-bromo-4H-pyrrolo [3,2-d]selenazole-5- carboxylic acid; |
| 63 | 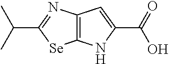 | 2-isopropyl-4H- pyrrolo[3,2- d]selenazole-5- carboxylic acid; |
| 64 | 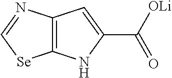 | lithium 4H-pyrrolo [3,2-d][1,3] selenazole-5- carboxylate; |
| 65 | 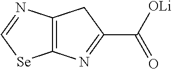 | lithium 6H-pyrrolo [3,2-d][1,3] selenazole-5- carboxylate; |
| 66 | 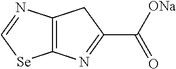 | sodium 6H-pyrrolo [3,2-d][1,3] selenazole-5- carboxylate; |
| 67 | 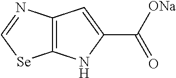 | sodium 4H-pyrrolo [3,2-d][1,3] selenazole-5- carboxylate; |
| 68 | 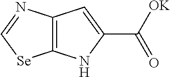 | potassium 4H- pyrrolo[3,2- d][1,3]selenazole-5- carboxylate; or |
| 69 | 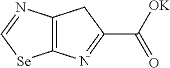 | potassium 6H- pyrrolo[3,2- d][1,3]selenazole-5- carboxylate. |

### D-Amino Acid Oxidase Related Assays

The activity of the compounds of the present disclosure toward DAO, can be determined from the methodologies discussed in following examples.

### Example 3

### Inhibition of Porcine Kidney DAO

Porcine kidney D-amino acid oxidase (catalog #A-5222 from Sigma) and D-serine (catalog #S-4250 from Sigma) is used to test the DAO inhibitory activity of test compounds The breakdown of D-serine by DAO produces hydrogen peroxidase, which can be measured using, for example, the Amplex® Red Hydrogen Peroxide Assay Kit (Catalog #A-22188, Molecular Probes, Inc.; Eugene, Oreg.). A working solution is prepared by mixing: distilled water (7.93 mL), sodium phosphate buffer (1 mL, 0.25M, pH 7.4), D-serine solution (1.0 mL, 100 mM in water), horseradish peroxidase (0.02 mL, 100 U/mL in buffer), and Amplex Red solution (0.05 mL, 1 mg dye in 200 μL in DMSO (50 μM in DMSO)). A working enzyme solution is prepared by diluting a D-amino acid oxidase stock solution (65 U/mL) four hundred fold. The working solution (99 μl) is transferred to wells of a Microfluor microtiter plate and a solution of the inhibitor in DMSO (1 μL) is added. The working enzyme solution (20 μl) is added to each well and the rate of reaction (hydrogen peroxide released) is determined by measuring the oxidation of Amplex Red by spectrophotometry, using a plate reader (excitation wavelength 544 nm, emission wavelength, 590 nM) after a reaction time of 15 minutes. Controls are carried out using DMSO in the absence of inhibitor. A known DAO inhibitor, indole-2-carboxylic acid, is used as a control in this assay.

### Example 4

### Inhibition of Human DAO

Human D-amino acid oxidase extracts are prepared by harvesting HEK293 cells either transiently or stably transfected with the human DAO clone (huDAO). The stable huDAO cell line is generated by co-transfecting the huDAO gene (Catalog#TC118941, Origene, Rockville, Md.) along with pcDNA3.1 (Invitrogen, Carlsbad, Calif.) at a 100:1 ratio into HEK293 cells under G418 selection. Transient huDAO transfections are implemented using Lipofectamine 2000 (Invitrogen, Carlsbad, Calif.) and following the manufacturer's protocol with the following specifics. HEK293 cells are seeded at 2×107 cells per T150 flask the day before transfection. huDAO DNA (Catalog#TC118941, Origene, Rockville, Md.) is transfected at 37.5 ug per flask and at a 3:1 DNA/Lipofectamine ratio. The DNA/Lipofectamine mixture is incubated on the cells for 48 hrs before cell harvesting. Similar results are obtained with transiently vs stably expressed huDAO. Extracts are harvested as follows. Culture liquid is removed from flasks and replaced with Hank's Buffered Saline Solution (20 mL). The cells are scraped into the Hank's Buffer and then transferred to a fresh tube. Samples are spun for 10 minutes at 3,000 rpm. The supernatant is decanted and the pellet resuspended in 50 mM Tris-HCL pH8.7, 1 μM FAD and 1 mM DTT, 20% glycerol (1 mL). Samples are then homogenized on ice for 20 seconds. Homogenates are spun down for 5 minutes at 3,000 rpm. The supernatants are removed and set aside. The pellets are resuspended in 50 mM Tris-HCL pH8.7, 1 μM FAD, 1 mM DTT and 0.1% octyl-β-D-glucoside, 20% glycerol (1 mL) and homogenized on ice for 20 seconds. Homogenates are spun for 5 minutes at 3,000 rpm. The supernatants are collected and combined with previously collected supernatants for a master stock. Extracts are then serially diluted and tested in the D-amino acid oxidase enzyme assay to determine activity based on protein concentration. Stocks are prepared accordingly, typically, for a twenty fold dilution in future assays.

Human D-amino acid oxidase (HEK293 cells stably transfected with huDAO clone) and D-serine (catalog #S-4250 from Sigma) are used to test the DAO inhibitory activity of test compounds. The breakdown of D-serine by DAO produces hydrogen peroxidase, which can be measured using, for example, the Amplex® Red Hydrogen Peroxide Assay Kit (Catalog #A-22188, Molecular Probes, Inc.; Eugene, Oreg.). A working solution is prepared by mixing: distilled water (7.93 mL), sodium phosphate buffer (1 mL, 0.25M, pH 7.4), D-serine solution (1.0 mL, 100 mM in water), horseradish peroxidase (0.02 mL, 100 U/mL in buffer), and Amplex Red solution (0.05 mL, 1 mg dye in 200 μL in DMSO (50 μM in DMSO)). A working enzyme solution is typically prepared by diluting a D-amino acid oxidase stock solution twenty fold. The working solution (99 μl) is transferred to wells of a Microfluor microtiter plate and a solution of the inhibitor in DMSO (1 μL) is added. The working enzyme solution (20 μl) is added to each well and the rate of reaction (hydrogen peroxide released) is determined by measuring the oxidation of Amplex Red by spectrophotometry, using a plate reader (excitation wavelength 544 nm, emission wavelength, 590 nM) after a reaction time of 15 minutes. Controls are carried out using DMSO (vehicle only, negative control) in the absence of inhibitor. A known DAO inhibitor, indole-2-carboxylic acid, is used as a positive control in this assay.

### Example 5

### DAO Whole Cell Assay 1—Toxicity

Human D-amino acid oxidase (huDAO) and D-serine (catalog #S-4250 from Sigma) are used to test the DAO inhibitory activity of test compounds. A stable hDAO cell line is generated by co-transfecting the huDAO gene (Catalog #TC118941, Origene, Rockville, Md.) along with pcDNA3.1 (Invitrogen, Carlsbad, Calif.) at a 100:1 ratio into HEK293 cells under G418 selection. The intracellular breakdown of D-serine by DAO produces hydrogen peroxide, which induces toxicity to the cell monolayer. This toxicity is measured by, for example, the AlamarBlue™ Reagent (Catalog #BUF012B, AbD Serotec Ltd., Kidlington, Oxford, UK). On day 1 of the assay, the following additions are made, in order, to a black, clear bottom, tissue culture treated 96-well plate (Corning #3904): 2 μL inhibitor (100× in 100% DMSO, or vehicle), 100 μL 70 mM D-serine in HEK media (DMEM/10% FBS), 100 μL huDAO cells (2×10⁵/ml). The cells are incubated for 18-24 hrs at 37° C./5% CO2. On day 2 of the assay, 20 μL of AlamarBlue™ Reagent is added to each well, and the plate is returned to the incubator for another 24 hrs. On day 3 of the assay, the amount of cellular toxicity (induced by hydrogen peroxide produced by intracellular huDAO) is determined by measuring the conversion of AlamarBlue reagent in a fluorescent plate reader (excitation wavelength 545 nm, emission wavelength, 590 nM; 37° C.).

### Example 6

### DAO Whole Cell Assay 2—Amplex Red

Human D-amino acid oxidase (huDAO) and D-serine (catalog #S-4250 from Sigma) are used to test the DAO inhibitory activity of test compounds. A stable huDAO cell line is created by co-transfecting the huDAO gene (Catalog #TC118941, Origene, Rockville, Md.) along with pcDNA3.1 (Invitrogen, Carlsbad, Calif.) into HEK293 cells under G418 selection. The intracellular breakdown of D-serine by huDAO produces hydrogen peroxide, which is measured by, for example, the Amplex® Red Hydrogen Peroxide Assay Kit (Catalog #A-22188, Molecular Probes, Inc.; Eugene, Oreg.). The following additions are made, in order, to a black, clear bottom, tissue culture treated 96-well plate (Corning #3904): 2 μL inhibitor (100× in 100% DMSO, or vehicle), 100 μL Detection Solution (30 mM D-serine, 20 uM Amplex Red, 0.05 U/mL HRP in Hanks Balanced Salt Solution/20 mM HEPES 7.4), and 100 μL huDAO cells (6×10⁵/ml). The intracellular huDAO activity is proportional to the rate of hydrogen peroxide produced by the cells and is determined by measuring the conversion of Amplex Red in a fluorescent plate reader (excitation wavelength 544 nm, emission wavelength, 590 nM) at 37° C. over a 60 min kinetic read.

### Example 7

### Detection of D-Amino Acids in Serum and Urine

Serum and urine samples are obtained and immediately frozen in a −80° C. freezer before analysis. Serum and urine levels of D-amino acids (aspartate, glutamate, glycine, D-serine, L-serine) are determined by precolumn derivatization with N-tert-butyloxy-carbonyl-L-cycleine and o-phthaldialdehyde (Hashimoto et al. J Chromatogr (1992) 52:325-53) coupled with a mobile phase gradient of methanol and 100 mmol/L, pH 7.2 sodium acetate, and reverse phase C-18 column for high-pressure liquid chromatography separation with fluorescent detection at excitation wavelength of 433 nm and emission wavelength of 344 nm. The absolute concentrations of amino acids are determined by computer analysis (Maxima 820, Waters, Mass.) of peak height with internal and external standards. D-amino acid levels (e.g., D-serine) can be determined in the presence and absence of test compound.

### Example 8

### Detection of D-Amino Acids in Brain and Plasma

Brain and plasma samples are obtained and immediately frozen in a −80° C. freezer before analysis. Amino acids are extracted from plasma using a protein precipitation procedure while brains are homogenized under acidic conditions. Levels of D-amino acids (serine, alanine, leucine and proline) are determined by precolumn derivatization with Marfey's reagent (Fluoro-dinitrophenyl-L-alanine amide) (Berna M. J. and Ackermann B. L. (2006) J Chromatogr B; doi:10.1016/j.jchromb.2006.08.029) coupled with a mobile phase gradient of 15 mM ammonium acetate in a combination of water, methanol and acetonitrile on a reverse phase C-18 column for high-pressure liquid chromatography separation with mass spectrometry detection in the negative single ion reaction mode. The absolute concentrations of amino acids are determined by computer peak area ratio with internal standards. D-amino acid levels (e.g., D-serine) can be determined in the presence and absence of test compound.

### Example 9

### D-Serine Induced Nephrotoxicity

D-serine and D-propargylglycine have been associated with nephrotoxicity and induce one or more of glucosuria, aminoaciduria, proteinuria, and polyuria. Compounds which inhibit DAO activity may also control the production of toxic metabolites of D-amino acid oxidation (e.g., D-serine) such as hydrogen peroxide and ammonia. Hydrogen peroxide and concomitantly produced oxygen radicals may lead to nephrotoxicity. Compounds described herein can be evaluated for their ability to attenuate the nephrotoxicity associated with D-serine or D-propargylglycine administration in rats as described in Williams and Lock 2005 Toxicology: 207:35-48 and Maekawa et al. 2005 Chem Res Toxicol. 18:1678-1682.

### Example 10

### Measurements of NMDA Receptor Affinity

To measure the affinity of the compounds reported herein for D-serine's binding site on the NMDA receptor (also known as the “Glycine site” or the “strychnine-insensitive glycine site”), a radioligand-binding assay is performed with membranes prepared from rat cerebral cortex. The radioactive ligand is [³H]MDL105,519 ((E)-3-(2)-phenyl-2-carboxyethenyl)-4,6-di-chloro-1[3H]-indole-2-carboxylicacid), a known glycine site antagonist. The amount of radioactivity displaced by the compounds is assessed by scintillation counting. Non-specific binding is accounted for in the presence of 1 mM Glycine. Affinities are calculated from the values of % inhibition of specific [³H]MDL105,519 binding by the test compounds. Indole-2-carboxylic acid is used as a positive control. The assay is commercially available at MDS Pharma Services (catalog no. 232910).

### Example 11

### Animal Models of Psychosis

Animals are housed in a temperature-controlled environment with free access to food and water. Animals are allowed to become acclimatized to their new environment and are handled during 1 week before starting the experiment (to permit habituation to the investigator). All experiments are performed in a separate, quiet, light level, temperature-controlled and sound attenuated experimental room. On the test day, food and water are withdrawn during the experiment and immediately replaced after the experiment such that no animal will is without food or water for longer than 8 hours. Behavioral evaluation is observed in one or more of the following models.

### Example 12

### Stereotypical Behavior and Hyperactivity Induced by Psychotomimetic Drugs

Each animal is individually placed into plastic test cages and allowed to habituate to the cage for up to 30 minutes prior to testing. Following habituation, animals are administered a psychotomimetic drug (such as MK-801, PCP, etc) and are then immediately replaced into the test box for behavioral observation. The stereotyped behavior and general motor activity are scored by an observer and/or via a video camera/activity monitor for up to 90 minutes post-injection (Hashimoto et al., 2005 Brain Res 1033:210-5). The test cages are thoroughly wiped clean with alcohol followed by a spray water rinse and dried after each session. This removes any olfactory cues that a rodent may leave on the test cage surface. In some cases, no drug treatment, baseline locomotor activity measurements are taken up to 3 days prior to the test day in order to assess the natural motor activity of the animal.

Therefore, a typical study schedule for stereotyped behavior and hyperactivity progresses as follows: Animals are dosed with test compounds 1 hour prior to systemic injection of psychotomimetic drug and returned to their home cages. 30 minutes prior to behavioral testing, animals are placed in test cages to acclimate. Following habituation, animals are subcutaneously injected with a psychotomimetic drug, and placed back into their respective test cages. Behavior is recorded by an observer and/or video tracker for up to 90 minutes post injection. Following behavioral testing, animals are returned to their home cages. Animals are allowed a drug ishout period of one week and behavior is re-evaluated in a counterbalanced fashion. At experiment end, animals are euthanized by CO₂ inhalation or pentobarbital overdose (>120 mg/kg). When brain tissue collection is necessary in order to analyze levels of neurotransmitters and immediate early genes, decapitation is performed. If blood sampling is necessary, it is done at the study end, after all behavioral observation is complete. To sample blood, animals are under terminal anesthesia by isoflurane or pentobarbital and sampling takes place at the retro-orbital sinus by sterile pipet tip or by cardiac puncture with a sterile needle.

### Example 13

### Effects of Psychomimetics and Antipsychotics on Cognition (Prepulse Inhibition Model)

Startle reactivity is measured by startle chambers. Each chamber consists of a clear nonrestrictive plexiglass 8.2 cm diameter cylinder resting on a 12.5×25.5 cm platform inside a ventilated box. A high-frequency loudspeaker inside the chamber produces both a continuous background noise of 65 decibels (dB) and a range of acoustic dB stimuli. Vibrations of the Plexiglass cylinder caused by the whole-body startle response of the animal are transduced into analog signals by a transduction unit attached to the platform. The signals are saved to a computer. The PPI test session generally consists of a randomized presentation of startle trials (120 dB pulse), prepulse trials (60-90 dB prepulse immediately preceding a 120 dB pulse) and no stimulus trials. This session usually lasts for 15-20 minutes. The acoustic stimuli are not harmful to the animals' hearing.

Therefore, a typical study schedule for PPI may progress as follows: Animals are dosed with test compounds or antipsychotic drugs (i.p. or s.c.). Immediately after this injection, animals are given a systemic injection (i.p. or s.c.) of either vehicle or psychotomimetic drug and 10 minutes later they are placed individually into startle chambers. A 65 dB background noise level is presented for a 10 minute acclimation period and then the PPI test session (consists of a presentation of startle trials (120 dB pulse), prepulse trials (60-90 dB prepulse immediately preceding a 120 dB pulse) and no stimulus trials) begins and lasts for 15 minutes. At the end of the test session, the animals are returned to their home cages. A no treatment, baseline measurement test session may occur up to 5-7 days prior to the drug treated test session. Following behavioral testing, animals are returned to their home cages. Animals are allowed a drug ishout period of one week and behavior is re-evaluated in a counterbalanced fashion. Geyer et al. (2001) Psychopharmacology 157(2-3) 117-154 review the use of PPI models in the study of schizophrenia.

### Example 14

### Forced Swim Model of Depression

Compounds described herein can be screened for the ability to alleviate the depression induced in a rodent forced swim model. Examples of such protocols are found in Porsolt et al. 1977 Arch Int Pharmacodyn Ther. 229:327-336 and Porsolt et al. 1979 Eur J. Pharmacol. 57:201-210.

In this model the animal is placed in plexiglass cylinder containing water from which there is no obvious means of escape. The animal alternates between vigorous swimming and immobility. The periods of immobility represent a state of despair in the animals. Animals dosed with known anti-depressants show a decrease in the duration of immobility. Periods of immobility are measured by an observer with a stop watch.

### Example 15

### Tail Suspension Model of Depression

A test for the screening of anti-depressant compounds is the tail suspension test. An example of the protocol can be found in Steru et al. 1985 Psychopharmacology 85:367-370.

This model, like the forced swim model, places animals in a situation that results in alternating vigorous movement and periods of immobility. In the assay, animals are suspended by their tails away from other objects and the floor. Like the forced swim test, animals treated with known anti-depressants show a decrease periods of immobility. These periods of immobility are measured by an observer with a stop watch.

### Example 16

### Animal Models for Assessing Memory and Cognitive Ability

In human patients there are a number of tests that can be used to measure cognitive ability. Useful test include Mini-Mental State Examination (MMSE), Alzheimer's Disease Assessment Scale (ADAS), Boston Naming Test (BNT), and Token Test (TK). The test scores are generally analyzed by determining the percent increase or decrease over the test period compared to the baseline score at the beginning of the test period. These tests and others can be used to assess the effectiveness of the agents used for the treatment or prevention of cognitive impairment.

In analyzing candidate memory protective agents it can be useful to measure the effect of a test compound on the cognitive ability in an animal model. There are a wide range of such tests that can be used to assess candidate compounds.

One useful test involves the assessment of working memory/attention in mice. Briefly, the effect of a compound on spatial working memory can be characterized in aged mice (i.e. about 25 months old) and in young mice (i.e. about 3 months old). The working memory of the mice can first be compromised by pharmacological means (i.e. scopolamine-induced impairment).

Working memory is the temporary storage of information (Bontempi et al. 2001 J Pharm and Exp Therap 299:297), and has been shown to be the primary type of memory disrupted in Alzheimer's disease, stroke and aging (Glasky et al. 1994 Pharm, Biochem and Behavior 47:325). Another useful test for assessing working memory measures Spontaneous Alternation behavior in mice. Spontaneous alternation is defined as the innate tendency of rodents to alternate free choices in a T-maze over a series of successive runs (Dember and Fowler 1958 Psychological Bulletin 55:412). This is a sequential procedure that relies on working memory because the ability to alternate requires that the animal retain specific information, which varies from trial to trial (Bontempi et al. 2003 Neuropsychopharmacology Apr. 2, 2003, 1-12). This test is also sensitive to varying parameters, such as delay intervals and increased number of trials, as well as pharmacological treatments affecting memory processes (Stefani and Gold, 2001 Journal of Neuroscience 21:609). In conducting this test, mice are first allowed to briefly explore a T-maze to become familiar with the apparatus. On the following day, a mouse is placed in a start box that is connected to the main stem of the T-maze. The elapsed time between the opening of the start box and the choice of an arm is measured (choice latency). The mouse is confined in the chosen arm for a set amount of time (e.g., 30 seconds) and then returned to the start box for the remaining consecutive trials in a testing session (Bontempi et al, 2003). Working memory performance for each mouse is assessed by the percentage of alternation over the trials in the testing session. Percentage is defined as entry in a different arm of the T-maze over successive trials.

The Delayed Non-Matching to Place (DNMTP) test is another useful animal model for testing the effect of a compound on cognitive ability. In this test, mice are trained and tested in an elevated eight-arm radial maze (Levin E. and Caldwell, D P (2006) Neurobiol Learn and Memory 86(1) 117-122) with a central start box placed in the center of a room with various pictures/objects placed around the room to serve as spatial cues. Each arm has a food pellet cup located at it far end. Food-deprived animals are habituated to the apparatus with all arms open and baited over a couple of successive daily free exploration periods prior to the test day. The exploration period ceases when all arms are visited and all food pellets are consumed (Bontempi et al 2001 (supra), 2003 (supra)). Animals are then trained to the DNMTP rule. A session consists of multiple trials that are separated by a defined interval. A trial consists of a study phase (two forced runs) and a test phase (two choice runs). In the study phase, the animal is given two consecutive forced runs in two different open arms. A forced run is when one arm of the maze opens allowing the animal to travel down to collect the food pellet and return to the central start box. After the second forced run, the test phase ensues. Two doors open simultaneously to begin the first choice run. One door reveals the first arm visited during the study phase and the other is an adjacent unvisited arm. Once the animal makes a choice and then returns to the start box, the next pair of doors opens (second choice run). The second choice run consists of the second arm visited in the study phase and an adjacent novel arm. During the choice runs, the animal is reinforced only when it enters the arm that had not been previously visited during the study phase. This is the non-matching to place rule; the rule being not to return to a previously visited arm. Once a mouse is trained to the DNMTP rule, variable delay periods between the study and test phases can be introduced. Mice are allowed to adapt to the delay paradigm over a few consecutive days prior to compound testing. Compound testing is conducted over a several consecutive days followed by a washout period with no paradigm training, followed by a vehicle injection for measurement of baseline performance. Test compound or vehicle injections are acutely administered prior to the start of each testing session. Working memory is evaluated by the comparison of performance on drug days versus baseline days. The effects of putative cognitive enhancing drugs are commonly evaluated in the delayed non-matching to position task (Crawley, What's Wrong With My Mouse? Behavioral Phenotyping of Transgenic and Knockout Mice, Wiley-Liss, New York, 2000). The DNMTP task is similar to schedule-induced operant tasks which include delayed matching and delayed non-matching to position tests in automated chambers, generally used in rats (Bontempi et al., 2001 (supra); Crawley, 2000 (supra)).

In addition to those working memory assays described above, another useful animal model to assess cognitive performance is the novel object recognition (NOR) assay (Ennaceur & Delacoer 1988, Behavioral Brain Res. 31, 47-49). Briefly, this assay assesses the ability of rodents to retain the memory of a “familiar” object by initially exposing them to the “familiar” object and then, after some period of time, exposing the rodent to both the “familiar” and a “novel” object. If the rodents recognize the “familiar object they will spend more time exploring the “novel” object more. If the memory of the “familiar” object is lost, rodents will investigate both objects equally. Test compounds are assessed for their ability to prolong the time period for which rodents can retain the memory of the familiar object (as measured by exploration of the novel).

Working memory tests such as those described above are thought to require identification and use of novel information on each trial (predominately affecting attentional processes) whereas spatial reference memory tasks require the same information to be used across trials.

The Morris Water Maze Task (D'Hooge and De Deyn (2001) Brain Res Rev 36 (1) 60-90) is a spatial navigation task in which an animal uses visual clues to swim to a hidden platform. Animals are motivated to find the fastest, most direct route to the platform in order to escape the water. The test typically consists of pre-training to a visible platform to test the animal's ability to conduct the procedural component of the task. Training for location of a hidden platform follows visible platform acquisition. Finally, a probe trial tests the animal's ability to find the spatial location that previously contained the hidden platform. Successful performance on the probe trial means that the animal spends significantly greater time in the trained quadrant versus non-trained quadrants. A deficit in learning and memory is defined as normal performance in the visible platform task but impaired performance on the hidden platform task.

Other tests, such as avoidance tasks, have been extensively used in the screening of compounds for cognitive enhancement (Crawley, 2000; Sarter et al. 1992 Psychopharmacology 107:461). For example, in the passive avoidance task, an animal is placed in a shuttle box containing a light and dark chamber (the dark is the natural preference of the rodent). The animal is trained to associate footshock with the properties of the natural preferred dark chamber. The next day, the animal is placed in the light chamber and latency to enter the dark chamber assesses the memory for the aversive association (Crawley, 2000). Potential drawbacks from these tests are that procedural components (the ability to acquire, store or retrieve memories) cannot be differentiated form declarative memory (remembering a specific item of information) as opposed to the Morris Water Maze task. Latency to enter the dark chamber on the first day is the only inherent control parameter in the avoidance task. It is known that the passive avoidance task can be affected by fear because an animal is negatively affected by the footshock so the test is often used to complement other learning and memory assays (Yamaguchi et al. 2001 Jpn Journal of Pharmacology 87:240).

Tests of cognitive ability are generally used in conjunction with tests designed to rule out artifacts that would impair the animal from performing complex tasks. For example, general effects on motor function (hyperactivity or sedation) can be measured by testing locomotor activity, including stereotypy (Crawley, 2000 (supra)). Motor coordination and balance can be assessed by assays such as the rotarod test. This test requires a mouse to continuously walk forward on a rotating cylinder to keep from falling off (Crawley, 2000 (supra)).

The disclosures of all articles and references mentioned in this application, including patents, are incorporated herein by reference in their entirety.

The present disclosure and the manner and process of making and using it, are now described in such full, clear, concise and exact terms as to enable any person skilled in the art to which it pertains, to make and use the same. It is to be understood that the foregoing describes certain embodiments of the present disclosure and that modifications may be made therein without departing from the spirit or scope of the present disclosure as set forth in the claims. To particularly point out and distinctly claim the subject matter of the present disclosure, the following claims conclude this specification.

1-255. (canceled)

256. A compound of the formula: 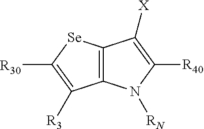
or a pharmaceutically acceptable salt thereof, wherein
R_{N} is (i) hydrogen;
(ii) C₁-C₆ alkylcarbonyl optionally substituted by one or two amino groups;
(iii) —S(O)ₙR₆, —S(O)ₙNH₂, —S(O)ₙNH(R₆), or —S(O)ₙN(R₆)₂, wherein each R₆ is independently C₁-C₆ alkyl, C₂-C₆alkenyl, or C₂-C₆ alkynyl, wherein each R₆ is optionally substituted with phenyl, and n is 1 or 2;
(iv) C₁-C₆ alkyl optionally substituted with one or more groups which are independently halogen or hydroxy;
(v) aryl(C₁-C₂)alkyl; or
(vi) heteroaryl(C₁-C₂)alkyl,
wherein the aryl and heteroaryl groups in (v) and (vi) are optionally substituted with one or more groups which are independently halogen, hydroxy, amino, nitro, C₁-C₆ alkylthio, hydroxy(C₁-C₆)alkyl, amino(C₁-C₆)alkyl, halo(C₁-C₆)alkyl, halo(C₁-C₆)alkoxy, or mono- or di(C₁-C₆)alkylamino;
R₄₀ is —COR or —R₅₀, wherein
R is hydroxy, hydroxyamino, C₁-C₆ alkoxy, C₁-C₆ alkylcarbonyloxy, aryloxy, aryl(C₁-C₆)alkoxy, or —NR₁R₂, wherein R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or more groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, C₅-C₇ heterocycloalkyl, mono- or di(C₁-C₆)alkylamino, or carboxy; and
R₅₀ is 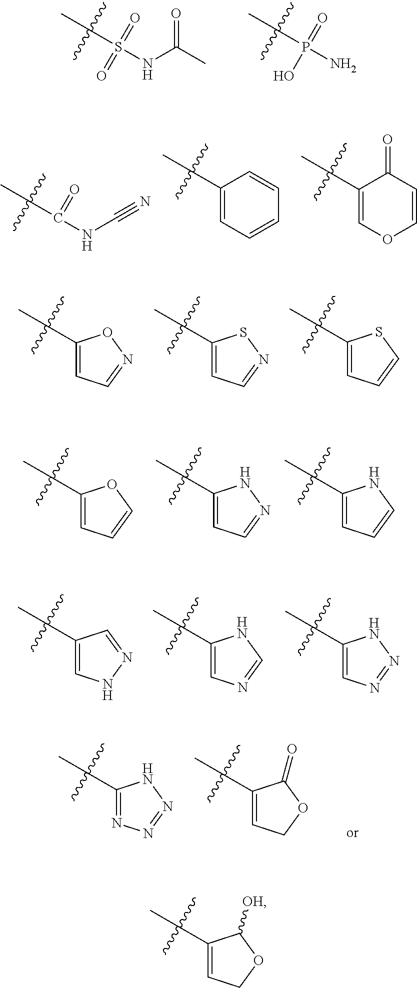
any of which may be optionally substituted with one or more groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy;
X is hydrogen, hydroxy, fluoro, chloro, bromo, cyano, nitro, C₁-C₆ alkyl, C₂-C₆ alkenyl, C₂-C₆ alkynyl, C₁-C₆ alkoxy, trifluoromethyl, or trifluoromethoxy;
R₃ is R₁₇ or R₁₈;
R₃₀ is R₁₈;
R₁₇ is —SR₅, —S(O)ₚR₅, —S(O)ₚNH₂, —S(O)ₚNHR₅, —S(O)ₚN(R₅)₂, COOH, C₁-C₁₀ alkyl, C₂-C₁₀ alkenyl, C₂-C₁₀ alkynyl, C₁-C₆ alkoxy, (C₁-C₆)alkylthio, halo(C₁-C₆)alkyl, halo(C₁-C₆)alkoxy, halo(C₁-C₆)alkylthio, or mono- or di(C₁-C₆)alkylamino, C₃-C₇ cycloalkyl, aryl, heteroaryl, aryl(C₁-C₆)alkyl, heteroaryl(C₁-C₆)alkyl, or C₅-C₇ heterocycloalkyl, wherein
each cycloalkyl, heterocycloalkyl, aryl, and heteroaryl group is optionally substituted with one or more groups which are independently halogen, hydroxy, hydroxyamino, amino, nitro, cyano, C₁-C₆ alkyl, C₂-C₆ alkenyl, C₂-C₆ alkynyl, C₁-C₆ alkoxy, C₁-C₆ alkylthio, hydroxyalkyl, halo(C₁-C₃)alkyl, halo(C₁-C₃)alkoxy, halo(C₁-C₃)alkylthio, mono- or di(C₁-C₆)alkylamino, or amino(C₁-C₆)alkyl;
R₅ is C₁-C₆ alkyl, C₂-C₆ alkenyl, or C₂-C₆ alkynyl, wherein R₅ is optionally substituted by phenyl, and
p is 1 or 2; and
each R₁₈ is independently hydrogen, halogen, hydroxy, amino, hydroxyamino, nitro, cyano, —SR₅₁, —S(O)ₜR₅₁, —S(O)ₜNH₂, —S(O)ₜNHR₅₁, —S(O)ₜN(R₅₁)₂, —Si(R₅₁)₃, C₁-C₄ alkyl, C₂-C₄ alkenyl, C₂-C₄ alkynyl, alkoxy, (C₁-C₄)alkylthio, halo(C₁-C₄)alkyl, halo(C₁-C₄)alkoxy, halo(C₁-C₄)alkylthio, or mono- or di(C₁-C₄)alkylamino, wherein
each R₅₁ is independently C₁-C₄ alkyl, C₂-C₄ alkenyl, or C₂-C₄ alkynyl; and
each t is independently 1 or 2; and
provided that the compound is not
(a) 4H-selenopheno[3,2-b]pyrrole-5-carboxylic acid;
(b) methyl 4H-selenopheno[3,2-b]pyrrole-5-carboxylate; and
(c) ethyl 4H-selenopheno[3,2-b]pyrrole-5-carboxylate.

257. The compound according to claim 256, wherein R₄₀ is —COR and R is hydroxy.

258. The compound according to claim 256, wherein X is hydrogen.

259. The compound according to claim 256, wherein R₃₀ is hydrogen.

260. The compound according to claim 256, wherein R₃ is hydrogen.

261. A salt of a compound according to claim 256, wherein the salt is:
lithium 4H-selenopheno[3,2-b]pyrrole-5-carboxylate;
lithium 6H-selenopheno[3,2-b]pyrrole-5-carboxylate;
sodium 4H-selenopheno[3,2-b]pyrrole-5-carboxylate;
sodium 6H-selenopheno[3,2-b]pyrrole-5-carboxylate;
potassium 4H-selenopheno[3,2-b]pyrrole-5-carboxylate; or
potassium 6H-selenopheno[3,2-b]pyrrole-5-carboxylate.

262. A compound of the formula: 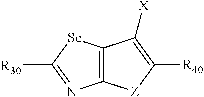
or a pharmaceutically acceptable salt thereof, wherein
Z is —N(R_{N})— or —S—, wherein
R_{N} is (i) hydrogen;
(ii) C₁-C₆ alkylcarbonyl optionally substituted by one or two amino groups;
(iii) —S(O)ₙR₆, —S(O)ₙNH₂, —S(O)ₙNH(R₆), or —S(O)ₙN(R₆)₂, wherein each R₆ is independently C₁-C₆ alkyl, C₂-C₆ alkenyl, or C₂-C₆ alkynyl, wherein each R₆ is optionally substituted with phenyl, and n is 1 or 2;
(iv) C₁-C₆ alkyl optionally substituted with one or more groups which are independently halogen or hydroxy;
(v) aryl(C₁-C₂)alkyl; or
(vi) heteroaryl(C₁-C₂)alkyl,
wherein the aryl and heteroaryl groups in (v) and (vi) are optionally substituted with one or more groups which are independently halogen, hydroxy, amino, nitro, C₁-C₆ alkylthio, hydroxy(C₁-C₆)alkyl, amino(C₁-C₆)alkyl, halo(C₁-C₆)alkyl, halo(C₁-C₆)alkoxy, or mono- or di(C₁-C₆)alkylamino;
R₄₀ is —COR or —R₅₀, wherein
R is hydroxy, hydroxyamino, C₁-C₆ alkoxy, C₁-C₆ alkylcarbonyloxy, aryloxy, aryl(C₁-C₆)alkoxy, or —NR₁R₂, wherein R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or more groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, C₅-C₇ heterocycloalkyl, mono- or di(C₁-C₆)alkylamino, or carboxy; and
R₅₀ is 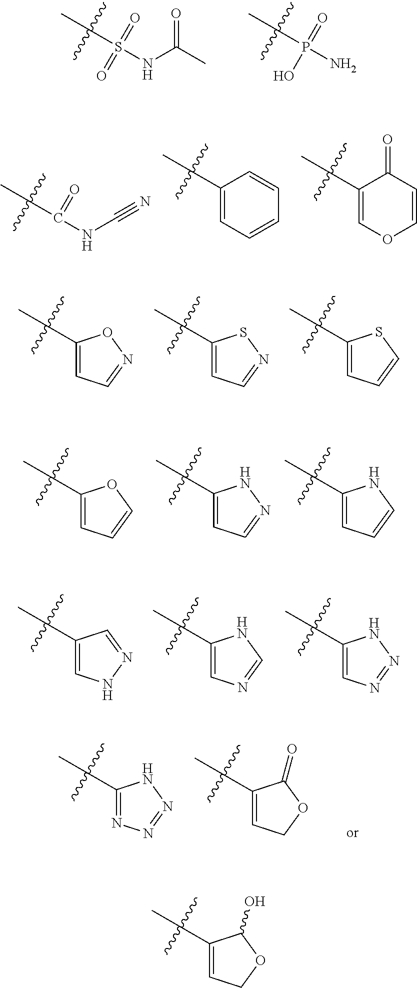
any of which may be optionally substituted with one or more groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy;
X is hydrogen, hydroxy, fluoro, chloro, bromo, cyano, nitro, C₁-C₆ alkyl, C₂-C₆ alkenyl, C₂-C₆ alkynyl, C₁-C₆ alkoxy, trifluoromethyl, or trifluoromethoxy; and
R₃₀ is hydrogen, halogen, hydroxy, amino, hydroxyamino, nitro, cyano, —SR₅, —S(O)ₚR₅, —S(O)ₚNH₂, —S(O)ₚNHR₅, —S(O)ₚN(R₅)₂, —Si(R₅₁)₃, C₁-C₄ alkyl, C₂-C₄ alkenyl, C₂-C₄ alkynyl, C₁-C₄ alkoxy, (C₁-C₄)alkylthio, halo(C₁-C₄)alkyl, halo(C₁-C₄)alkoxy, halo(C₁-C₄)alkylthio, or mono- or di(C₁-C₄)alkylamino, wherein
R₅ is C₁-C₄ alkyl, C₂-C₄ alkenyl, or C₂-C₄ alkynyl; and
p is 1 or 2.

263. The compound according to claim 262, wherein R₄₀ is —COR and R is hydroxy.

264. The compound according to claim 262, wherein X is hydrogen.

265. The compound according to claim 262, wherein R₃₀ is hydrogen.

266. The compound according to claim 262, wherein Z is —N(R_{N})— and R_{N} is hydrogen.

267. The compound according to claim 262, wherein Z is —S—.

268. A compound of the formula: 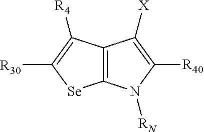
or a pharmaceutically acceptable salt thereof, wherein
R_{N} is (i) hydrogen;
(ii) C₁-C₆ alkylcarbonyl optionally substituted by one or two amino groups;
(iii) —S(O)ₙR₆, —S(O)ₙNH₂, —S(O)ₙNH(R₆), or —S(O)ₙN(R₆)₂, wherein each R₆ is independently C₁-C₆ alkyl, C₂-C₆ alkenyl, or C₂-C₆ alkynyl, wherein each R₆ is optionally substituted with phenyl, and n is 1 or 2;
(iv) C₁-C₆ alkyl optionally substituted with one or more groups which are independently halogen or hydroxy;
(v) aryl(C₁-C₂)alkyl; or
(vi) heteroaryl(C₁-C₂)alkyl,
wherein the aryl and heteroaryl groups in (v) and (vi) are optionally substituted with one or more groups which are independently halogen, hydroxy, amino, nitro, C₁-C₆ alkylthio, hydroxy(C₁-C₆)alkyl, amino(C₁-C₆)alkyl, halo(C₁-C₆)alkyl, halo(C₁-C₆)alkoxy, or mono- or di(C₁-C₆)alkylamino;
R₄₀ is —COR or —R₅₀, wherein
R is hydroxy, hydroxyamino, C₁-C₆ alkoxy, C₁-C₆ alkylcarbonyloxy, aryloxy, aryl(C₁-C₆)alkoxy, or —NR₁R₂, wherein R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or more groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, C₅-C₇ heterocycloalkyl, mono- or di(C₁-C₆)alkylamino, or carboxy; and
R₅₀ is 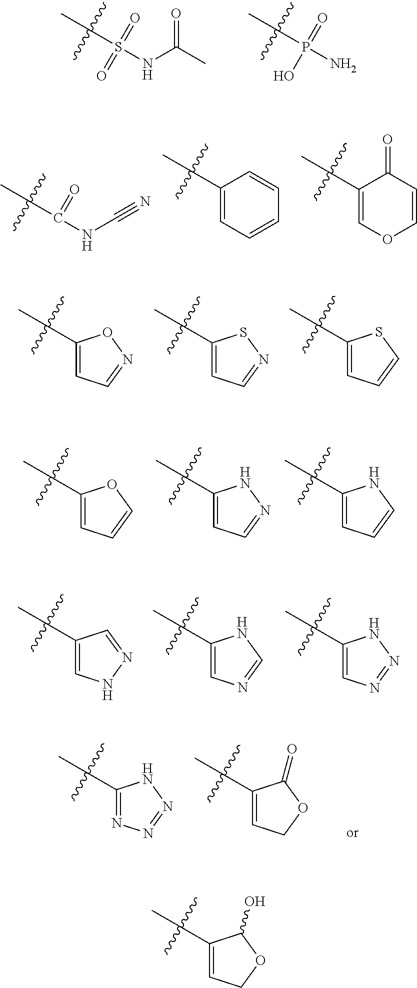
any of which may be optionally substituted with one or more groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy;
X is hydrogen, hydroxy, fluoro, chloro, bromo, cyano, nitro, C₁-C₆ alkyl, C₂-C₆ alkenyl, C₂-C₆ alkynyl, C₁-C₆ alkoxy, trifluoromethyl, or trifluoromethoxy;
R₄ is R₁₈ or R₁₇;
R₃₀ is R₁₈;
R₁₇ is —SR₅, —S(O)ₚR₅, —S(O)ₚNH₂, —S(O)ₚNHR₅, —S(O)ₚN(R₅)₂, COOH, C₁-C₁₀ alkyl, C₂-C₁₀ alkenyl, C₂-C₁₀ alkynyl, C₁-C₆ alkoxy, (C₁-C₆)alkylthio, halo(C₁-C₆)alkyl, halo(C₁-C₆)alkoxy, halo(C₁-C₆)alkylthio, or mono- or di(C₁-C₆)alkylamino, C₃-C₇ cycloalkyl, aryl, heteroaryl, aryl(C₁-C₆)alkyl, heteroaryl(C₁-C₆)alkyl, or C₅-C₇ heterocycloalkyl, wherein
each cycloalkyl, heterocycloalkyl, aryl, and heteroaryl group is optionally substituted with one or more groups which are independently halogen, hydroxy, hydroxyamino, amino, nitro, cyano, C₁-C₆ alkyl, C₂-C₆ alkenyl, C₂-C₆ alkynyl, C₁-C₆ alkoxy, C₁-C₆ alkylthio, hydroxyalkyl, halo(C₁-C₃)alkyl, halo(C₁-C₃)alkoxy, halo(C₁-C₃)alkylthio, mono- or di(C₁-C₆)alkylamino, or amino(C₁-C₆)alkyl;
R₅ is C₁-C₆ alkyl, C₂-C₆ alkenyl, or C₂-C₆ alkynyl, wherein each R₅ is optionally substituted by phenyl, and
p is 1 or 2; and
each R₁₈ is independently hydrogen, halogen, hydroxy, amino, hydroxyamino, nitro, cyano, —SR₅₁, —S(O)ₜR₅₁, —S(O)ₜNH₂, —S(O)ₜNHR₅₁, —S(O)ₜN(R₅₁)₂, —Si(R₅₁)₃, C₁-C₄ alkyl, C₂-C₄ alkenyl, C₂-C₄ alkynyl, C₁-C₄ alkoxy, (C₁-C₄)alkylthio, halo(C₁-C₄)alkyl, halo(C₁-C₄)alkoxy, halo(C₁-C₄)alkylthio, or mono- or di(C₁-C₄)alkylamino, wherein
each R₅₁ is independently C₁-C₄ alkyl, C₂-C₄ alkenyl, or C₂-C₄ alkynyl; and
each t is independently 1 or 2;
provided that
(a) the compound is not
(i) 6H-selenopheno[2,3-b]pyrrole-5-carboxylic acid;
(ii) ethyl 6H-selenopheno[2,3-b]pyrrole-5-carboxylate; and
(b) R₄₀ is not unsubstituted phenyl when R_{N} is methyl.

269. The compound according to claim 268, wherein R₄₀ is —COR and R is hydroxy.

270. The compound according to claim 268, wherein X is hydrogen.

271. The compound according to claim 268, wherein R₃₀ is hydrogen.

272. The compound according to claim 268, wherein R₄ is hydrogen.

273. A compound of the formula: 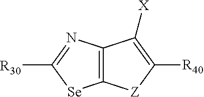
or a pharmaceutically acceptable salt thereof, wherein
Z is —N(R_{N})— or —S—, wherein
R_{N} is (i) hydrogen;
(ii) C₁-C₆ alkylcarbonyl optionally substituted by one or two amino groups;
(iii) —S(O)ₙR₆, —S(O)ₙNH₂, —S(O)ₙNH(R₆), or —S(O)ₙN(R₆)₂, wherein each R₆ is independently C₁-C₆ alkyl, C₂-C₆ alkenyl, or C₂-C₆ alkynyl, wherein each R₆ is optionally substituted with phenyl, and n is 1 or 2;
(iv) C₁-C₆ alkyl optionally substituted with one or more groups which are independently halogen or hydroxy;
(v) aryl(C₁-C₂)alkyl; or
(vi) heteroaryl(C₁-C₂)alkyl,
wherein the aryl and heteroaryl groups in (v) and (vi) are optionally substituted with one or more groups which are independently halogen, hydroxy, amino, nitro, C₁-C₆ alkylthio, hydroxy(C₁-C₆)alkyl, amino(C₁-C₆)alkyl, halo(C₁-C₆)alkyl, halo(C₁-C₆)alkoxy, or mono- or di(C₁-C₆)alkylamino;
R₄₀ is —COR or —R₅₀, wherein
R is hydroxy, hydroxyamino, C₁-C₆ alkoxy, C₁-C₆ alkylcarbonyloxy, aryloxy, aryl(C₁-C₆)alkoxy, or —NR₁R₂, wherein R₁ and R₂ are independently (i) hydrogen, (ii) C₁-C₆ alkyl, (iii) C₂-C₆ alkenyl, (iv) C₂-C₆ alkynyl, or (v) phenyl optionally substituted with one or more groups which are each independently halogen, hydroxy, amino, nitro, cyano, C₁-C₆ alkyl, C₂-C₆ alkenyl, C₂-C₆ alkynyl, C₁-C₆ alkoxy, C₁-C₆ alkylthio, halo(C₁-C₆)alkyl, halo(C₁-C₆)alkoxy, halo(C₁-C₆)alkylthio, C₃-C₇ cycloalkyl, C₅-C₇ heterocycloalkyl, mono- or di(C₁-C₆)alkylamino, or carboxy; and
R₅₀ is 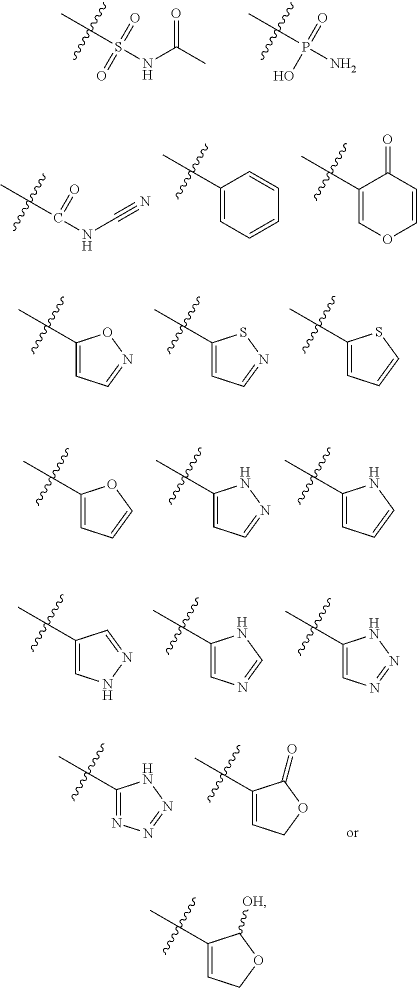
any of which may be optionally substituted with one or more groups which are each independently halogen, hydroxy, nitro, C₁-C₆ alkyl, C₂-C₆ alkenyl, C₂-C₆ alkynyl, halo(C₁-C₂)alkyl, or halo(C₁-C₂)alkoxy;
X is hydrogen, hydroxy, fluoro, chloro, bromo, cyano, nitro, C₁-C₆ alkyl, C₂-C₆ alkenyl, C₂-C₆ alkynyl, C₁-C₆ alkoxy, trifluoromethyl, or trifluoromethoxy; and
R₃₀ is hydrogen, halogen, hydroxy, amino, hydroxyamino, nitro, cyano, —SR₅, —S(O)ₚR₅, —S(O)ₚNH₂, —S(O)ₚNHR₅, —S(O)ₚN(R₅)₂, —Si(R₅₁)₃, C₁-C₄ alkyl, C₂-C₄ alkenyl, C₂-C₄ alkynyl, C₁-C₄ alkoxy, (C₁-C₄) alkylthio, halo(C₁-C₄)alkyl, halo(C₁-C₄)alkoxy, halo(C₁-C₄)alkylthio, or mono- or di(C₁-C₄)alkylamino, wherein
R₅ is C₁-C₄ alkyl, C₂-C₄ alkenyl, or C₂-C₄ alkynyl; and
p is 1 or 2.

274. The compound according to claim 273, wherein R₄₀ is —COR and R is hydroxy.

275. The compound according to claim 273, wherein X is hydrogen.

276. The compound according to claim 273, wherein R₃₀ is hydrogen.

277. The compound according to claim 273, wherein Z is —N(R_{N})— and R_{N} is hydrogen.

278. The compound according to claim 273, wherein Z is —S—.

279. A pharmaceutical composition comprising a therapeutically effective amount of a compound or salt according to claim 256 and a pharmaceutically acceptable excipient, diluent or carrier.

280. The pharmaceutical composition according to claim 279, further comprising one or more agents useful in the prevention and/or treatment of a neurological or psychiatric disorder.

281. A method of preventing and/or treating a neurological or psychiatric disorder comprising administering to a patient in need thereof a therapeutically effective amount of a compound according to claim 256.

282. The method of claim 281, wherein the neurological or psychiatric disorder is selected from schizophrenia, Alzheimer's disease, dementia, bipolar disorder, mood disorder, or depression.

283. The method of claim 282, wherein the dementia is senile dementia.

284. The method of claim 282, wherein the dementia is dementia associated with Alzheimer's disease.

285. The method of claim 281, wherein the compound, salt or composition is administered orally.

286. The method of claim 281, wherein the compound, salt or composition is provided as a sustained release formulation.

287. The method of claim 281, further comprising administering one or more agents useful in the prevention and/or treatment of a neurological or psychiatric disorder.

288. The method of claim 287, wherein the administering is performed simultaneously.

289. The method of claim 287, wherein the administering is performed sequentially.

290. The method of claim 281, wherein the patient has been medically diagnosed with a neurological or psychiatric disorder.
